SEQUENCE LISTING (85 sequences)

SEQ ID NO: 1 - DNA, 267 bp, Homo sapiens
tttgagaattgaaactttgatttattggctaggggagagagatgaaaagaggatcactctggtagcaggagttttgggcatgtccctctgagatccatttgatgaaaacattttttaaaacatggtagggcctcattttgcacattttcttcacggtgtcatcaaataaacatcatggtaaatataaacttgttcgcccgtcagaaagcggcgccgggagggagcgagcgtgagaaagtgagcgtgtgcgctgccggagagcacgac

SEQ ID NO: 2 - DNA, 581 bp, Homo sapiens
atatatccagtgctttgcaagtgctctgcgcaccttgtctcactccatcctgacaataatcctgggaggtgtgtgcaattactacgactactctcttttttatagatcattaaattcagaactaaggagttaagtaacttgtccaagttgttcacacagtgaagggaggggccaagatatgatggctgggagtctaattgcagttccctgagccatgtgcctttctcttcactgaggactgccccattcttgagtgccaaacgtcactagtaacagggtgtgcctagataatttatgatccaaactgagtcagtttggaaagtgaaagggaaacttacatataatccctccgggacaatgagcaaaaactaggactgtccccagacaaatgtgaacatacatatcatcacttaaattaaaatggctatgagaaagaaagagggggagaaacagtcttgcgggtgtgaagtcccatgaccagccatgtcaaaagaaggtaaagaagtcaagaaaaagccatgaagcccatttgatttcatttttctgaaaataggctcaagagggaataaattagaaactca

SEQ ID NO: 3 - DNA, 7635 bp, Homo sapiens
ggggccggggggcggagccttgcgggctggagcgaaagaatgcgggggctgagcgcagaagcggctcgaggctggaagaggatcttgggcgccgccaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 4 - DNA, 4833 bp, Homo sapiens
gtgaactgttgcaccgtgcaattgcacactataaatgtctttccttatctgtgtgtactcttatctcactgttctattttttctcctcatttatattaactctttcttacctttttttctgaacttctaggccttctctttccagaactggtggaagacaaatgaaacggccaagatggtaagaaacaagccgcatttctccttggggagactgataatttaaaaggtttgttgtgtcagaaacattcccagcttcatcaccaaccctttccttccacctctgcccactggagaccacttatatcccgaagcggacgcggcagctgaagtcaggaaaccatgcatcacattagcaggagccaactgcagactttaaactccgttcaacatgtggatgcggcagagaaatgacctgtccagacaagccggggcagctcataaactggttcatctgctccctgtgcgtcccgcgggtgcgtaagctctggagcagccggcgtccaaggacccggagaaaccttctgctgggcactgcgtgtgccatctacttgggcttcctggtgagccaggtggggagggcctctctccagcatggacaggcggctgagaaggggccacatcgcagccgcgacaccgccgagccatccttccctgagatacccctggatggtaccctggcccctccagagtcccagggcaatgggtccactctgcagcccaatgtggtgtacattaccctacgctccaagcgcagcaagccggccaatatccgtggcaccgtgaagcccaagcgcaggaaaaagcatgcagtggcatcggctgccccagggcaggaggctttggtcggaccatcccttcagccgcaggaagcggcaagggaagctgatgctgtagcacctgggtacgctcagggagcaaacctggttaagattggagagcgaccctggaggttggtgcggggtccgggagtgcgagccgggggcccagacttcctgcagcccagctccagggagagcaacattaggatctacagcgagagcgccccctcctggctgagcaaagatgacatccgaagaatgcgactcttggcggacagcgcagtggcagggctccggcctgtgtcctctaggagcggagcccgtttgctggtgctggaggggggcgcacctggcgctgtgctccgctgtggccctagcccctgtgggcttctcaagcagcccttggacatgagtgaggtgtttgccttccacctagacaggatcctggggctcaacaggaccctgccgtctgtgagcaggaaagcagagttcatccaagcagcagcagcagcgtgtctttccatgcgcttggcattctttattttcccagcctgggaggatatgagagttccagggaaatgctgtattggacatgcaagactcacctggggaacttatcagcagttgctgaaacagaaatgctggcagaatggccgagtacccaagcctgaatcgggttgtactgaaatacatcatcatgagtggtccaagatggcactctttgattttttgttacagatttataatcgcttagatacaaattgctgtggattcagacctcgcaaggaagatgcctgtgtacagaatggattgaggccaaaatgtgatgaccaaggttctgcggctctagcacacattatccagcgaaagcatgacccaaggcatttggtttttatagacaacaagggtttctttgacaggagtgaagataacttaaacttcaaattgttagaaggcatcaaagagtttccagcttctgcagtttctgttttgaagagccagcacttacggcagaaacttcttcagtctctgtttcttgataaagtgtattgggaaagtcaaggaggtagacaaggaattgaaaagcttatcgatgtaatagaacacagagccaaaattcttatcacctatatcaatgcacacggggtcaaagtattacctatgaatgaatgacaaaagaatcttctggctagggtgttagatatatttatgcatttttggttttgtttttaaatcaagcacatcaacctcaagcccgtttagcaatgaggcagtgtagatgaatacgtaaaataaatgactttaaccaagtagctataatgggacttagcactgtatgcatacttaaaaaggttttgaaaaacaaactacttgagaaatatttgtttatatttttctctaacatcatgctatgtgtcagtctgaacatctgacaacagaaatttcagttattattctagctaagttttgaaaacatttgtcatgctgtttaatagaaaactgcaaaccagagacactgactccattaataaaccatattttgtgccgttttgactgttctgaccaaatactaatgggaacaattcttgacgtttttctgttgctgattgttaacatagagcagtctctacactaccctgaggcaactctacattggaacactgaggcttacagcctgcaagagcatcagagctgaccatacatttaaacagaaatgctggtttatttgcaaaatcaccagtatattttctattgtgtctataaaaaatcagtcatttaagtacaagaatcatattttccattcctttttagaaatttattttgttgtccctatggaaatcattcacatctgacaatttatatgttaaagagttttactctctctattttggtccaatttgtatctagtggctgagaaattaaataattctaaagtatgaagttacctatctgaaaatgtacttacagagtatcattttaaaatggatgtctctttaaaaattttgttacttttaccaacaatgtaatataatttatgtatattttattaataatagtgaattccttaaaatttgttctatgtacttatatttaatttgatttaatggttactgcccagatattgagaattggttcaaatattgagtgtgtttcaatatattatctggcttatttcaacatgagtaatatgagcaaaataagttaaaacctgcgtctgatcaattttcctcatgactagaactaaaacagtaaatttggacaatattaagcctcaaataatcatctccaaactccttctaacactttttaaatcagattggaagacatggacaaatcaggttcatgtgttgcatctttatgtcctttgccaatatccaagatcatcacatatggtagatattcacatggagtttcaaattcagaatagattaccattaccttcctgcccttacacatcctactccttatttaaaagttctatttgtgacttttcatttcctgaaagtttaaaaatacaatttgagaatgtttataatacattctctcctgtcttttcacggttacgtctgttattgctgaaatacaccacattttctttgttctggtcaaggttaactcaatatctgtgtgaaagagaactactaacaacgttacaatagaggctagatttgaaaaaaaaaatctatagatctaattgatacaattgtagaacaaaatgtcaaaataatgttttaagtataagagaagatggaccaaggagagagagatcatttgaaaatctaattgtagcttttctaggctcacattcatgtactacttttagcacccttatgggctgtgctcgccccctggacagttgagctttggattatcttcctcttcaattttccctctattgacccgagtgtctccctctgcttctacagatttatagtactccttggctcttttgagtctccacttttactcactgtctctgggatttttaagatccttttcttctcttataaatcatcctcttaatgaaaattagcctaacaaaagtttggagactggaatcctactttgagccactgacttgaaataactcttttggcaagttgcctgacatcctgtcttaccaaggtggcatatttgcatttttactgcttaaaacatttttttttttttaccatctttatccaaatttatcatattgatggtaggactaacaggctttttagaagctggctttaactttgagtctcaagctacaatgctgttgggcagcctggtcttcccacgtgagggtttaactttgtttatttgcctccagttattccaaaatgcttattaaatgaaagtcccaggaacatgtttattttagtcacctttgctttttaacaattttgttttgtaatcaatgagtaattcatgatgaattatttttgactaatggatagccgaaggccaggcttttaattctaataggtaatgttcttcttttgtcttattgaaacaatgagaatactctgtgcatttcaaatgcactccgattatgctgtggttttattcacataagcacaatatgtgttttatttataacttcataacaaacttataatataataatttaccttagcagacatgcaaaagcttattcttgtgtgacttactttctttaagctaataatataaaaataaatatgtatcttaaaaatctataataaaacattagaaattaaagatatgtgctttttattttgcagatgagttcatttgcttttgtagatgtgttttcagagctaggtacagaggaatgtttgctacctttagcggtgaaaaaagaaagagagtcaagaattttgttggattgtgtttgtgtgtgcatatatttgatatcatcattatatttgtaatctttggacttgtaatcatagcctgtttattctactgtgccattaaatatactttaccttatacataacgaataaaatacctagaagtagatttatttacaaaaaaaaaaaaaaaa

SEQ ID NO: 5 - DNA, 2750 bp, Homo sapiens
gtgctctgagtttttggtttctgtttcaccttgtgtctgagctggtctgaaggctggttgttcagactgagcttcctgcctgcctgtaccccgccaacagcttcagaagaaggagcagcccctgggtgcgtccactttctgggcacgtgaggttgggccttggccgcctgagcccttgagttggtcacttgaaccttgggaatattgagattatattctcctgccttttaaaaagatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgatggtgctattttgtttgttttgttttgttttgtttttttgagacagaatctcgctctgtcgcccaggctggagtgcagtggcgtgatctcggctcaccgcaagctccgcctcccgggttcaggccattctcctgcctcagcctcccgagtagctgggactacaggggcccgccaccacacctggctaattttttaaaaatatttttagtagagacagggtttcactgtgttagccagggtggtcttgatctcctgacctcgtgatccacccacctcggcctcccaaagtgctgggattacaggcgtgagccaccgcgcctggccgatggtactatttagatataacactatgtttatttactaattttctagattttctactttattaattgttttgcacttttttataagagctaaagttaaataggatattaacaacaataacactgtctcctttctcttatgcttaaggctttgggaatgtttttagctggtggcaataaataccagacacgtacaaaatccagctatgaatatagagggcttatgattcagattgttatctatcaactataagcccactgttaatattctattaactttaattctctttcaaagctaaattccacactaccacattaaaaaaattagaaagtagccacgtatggtggctcatgtctataatcccagcactttgggaggttgaggtgggaggattgcttgaacccaagaggtcaaggctgcagtgagccatgttcacaccgctgcactcaagcttgggtgacagaacaagaccccgtctcaaaaaaaattttttttttaataaaacaaaatttgtttgaaatcttttaaaaattcaaatgatttttacaagttttaaataagctctccccaaacttgctttatgccttcttattgcttttatgatatatatatgcttggctaactatatttgctttttgctaacaatgctctggggtctttttatgcatttgcatttgctctttcatctctgcttggattattttaaatcattaggaattaagttatctttaaaatttaagtatcttttttcaaaaacattttttaatagaataaaatataatttgatcttattaaa

SEQ ID NO: 6 - DNA, 2938 bp, Homo sapiens
gagtgagtcatctctgttctgctttaggagtaaagtttaccctgcagttccttctgtggtgaagttttctctttctctcggagaccagattctgcctttctgctggagggaagtgttttcacaggttctcctccttttatcttttgtgttttttttcaagccctgctgaatttgctagtcaactcaacaggaagtgaggccatggagggaggcagaagagccagggtggttattgaaagtaaaagaaacttcttcctgggagcctttcccacccccttccctgctgagcacgtggagttaggcaggttaggggactcggagactgcgatggtgccaggaaagggtggagcggattatattctcctgccttttaaaaagatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaagagagaagcagcagccttgaaggaagtcctgatgaattttcaaatggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgatggtgctattttgtttgttttgttttgttttgtttttttgagacagaatctcgctctgtcgcccaggctggagtgcagtggcgtgatctcggctcaccgcaagctccgcctcccgggttcaggccattctcctgcctcagcctcccgagtagctgggactacaggggcccgccaccacacctggctaattttttaaaaatatttttagtagagacagggtttcactgtgttagccagggtggtcttgatctcctgacctcgtgatccacccacctcggcctcccaaagtgctgggattacaggcgtgagccaccgcgcctggccgatggtactatttagatataacactatgtttatttactaattttctagattttctactttattaattgttttgcacttttttataagagctaaagttaaataggatattaacaacaataacactgtctcctttctcttatgcttaaggctttgggaatgtttttagctggtggcaataaataccagacacgtacaaaatccagctatgaatatagagggcttatgattcagattgttatctatcaactataagcccactgttaatattctattaactttaattctctttcaaagctaaattccacactaccacattaaaaaaattagaaagtagccacgtatggtggctcatgtctataatcccagcactttgggaggttgaggtgggaggattgcttgaacccaagaggtcaaggctgcagtgagccatgttcacaccgctgcactcaagcttgggtgacagaacaagaccccgtctcaaaaaaaattttttttttaataaaacaaaatttgtttgaaatcttttaaaaattcaaatgatttttacaagttttaaataagctctccccaaacttgctttatgccttcttattgcttttatgatatatatatgcttggctaactatatttgctttttgctaacaatgctctggggtctttttatgcatttgcatttgctctttcatctctgcttggattattttaaatcattaggaattaagttatctttaaaatttaagtatcttttttcaaaaacattttttaatagaataaaatataatttgatcttattaaa

SEQ ID NO: 7 - DNA, 3220 bp, Homo sapiens
ctcccggagatgccccgcggcagccgcgctcggggctctaagagaaaaaggagttggaatacagaatgcccatcctttccaggagaaagaccactgcaggtcagaagagcaggtctcaggacagcaggggcagctgcctctctctctgaagcatggctcaggtgtggagaagggtttcagaacacttctgggaatccgtcattaacagctgaagagaagacgattacagaaaagcaccttgaattatgccctagacccaagcaagaaaccaccacatctaaaagcaccagtgggcttacagacataacatggagctccagtggaagtgatttgtcggatgaagataagacactttctcagttacagagagatgaattacagtttatcgactgggagattgacagtgacagggcagaggctagtgactgtgatgaatttgaagatgacgagggtgctgtggaaatctcagactgtgcttcttgtgcaagtaatcagtctttgacaagtgatgagaagctgtcggagcttcccaagccaagttctatagaaattttagagtattcatcagatagtgaaaaagaagatgatttggaaaatgtcctactcattgattcagaatcccctcacaaataccacgtgcagtttgcatcggatgcaagacagattatggagagactgatagatccaaggacaaaatcaacagagaccattttgcatacacctcagaaacccacagctaagtttcccaggactccagaaaattcagcaaagaagaagcttttaagaggtggactagcagaaagactaaatggactgcagaatcgagagagatctgctatttctttgtggagacatcaatgtatttcttaccaaaagacactttcaggtagaaaatctggtgtattaactgtgaaaattttagagctgcatgaggaatgtgccatgcaagttgccatgtgtgagcagttattggggtcaccagccaccagctcctcccaaagtgtggctcccaggcctggagctggcctgaaagttctcttcaccaaggagactgcaggctacctcaggggccgtccccaggacactgtccggatcttccctccctggcaaaaactgattattccaagtggaagttgccctgttattctgaatacttacttttgtgagaaagttgttgccaaagaagattcagaaaaaacttgtgaagtgtactgtccggacataccccttccaagaagaagcatctctttggcccagatgtttgtaattaagggtctaacaaataattcacctgaaatccaggttgtgtgtagtggtgtagccactacagggacagcctggacccatgggcacaaagaagcaaaacagcgcatcccaaccagcactcccctgagggattctctcctggatgtggtggaaagccagggagctgcctcgtggccaggagctggagtccgagtggtggtgcaaagagtgtattctcttcccagcagagacagcaccaggggtcagcagggggccagctcaggacacacagacccagctggaactcgagcctgccttctggtacaagatgcctgtggaatgttcggtgaagtgcacttggagttcaccatgtcgaaggcaagacagttggaagggaagtcttgcagcctggtgggaatgaaggttctacagaaagtcaccagaggaaggacagcggggattttcagtttgattgacaccctgtggcccccagcgatacctctgaaaacacctggccgcgaccagccctgtgaagagataaaaactcatctgcctcctccagccttgtgttacatcctcacagctcatccaaatctgggacaaattgatataattgacgaagaccccatttataagctttaccagcctccagttacccgctgcttaagagacattctccagatgaatgatcttggtacccgttgcagtttctatgccacggtgatttaccaaaaaccacagctgaagagtctgctgcttctggagcaaagggagatctggctgctagtgaccgatgtcactctgcaaacgaaggaggagagagaccccaggctccccaaaaccctgctggtctatgtggcccccttgtgtgtgctgggctctgaagtcctggaggcactcgctggggctgcccctcacagcctcttcttcaaggacgctctccgtgaccagggtcggattgtttgtgctgaacgaactgtcctcttgcttcagaagccccttttgagtgtggtctctggtgcaagttcctgtgagctgcctggcccggtgatgctcgacagcctggactctgcaacacctgtcaactccatctgcagtgttcaaggcactgtggttggcgtggacgagagcactgctttctcatggcctgtgtgtgacatgtgtggcaacgggagattggaacagaggccggaagacagaggcgccttttcctgtggggactgctcccgggtggtcacatctcctgttctcaagaggcacctgcaggtcttcctggactgccgctcaagaccgcagtgcagagtgaaggtcaagctgttgcagcgcagcatttcctccctgctgaggtttgccgccggtgaagatgggagctacgaagtgaagagtgtcctcggaaaggaagtggggttgttaaattgttttgtccagtccgtaaccgcccacccgaccagctgcattggattggaggaaatcgagcttctgagtgcaggaggggcctctgcagaacactagcggttgccgcaggatctgtgaactttgcaatgtggctgcaagggtggtggtggtggtggtgatttggggtagttatttgttaactatggacacagtgaacgtagtttacgatcttgaaatgaaacttagatttttctggggaaatgttcagatacagttttgtgaactgtaaatcaaaatacctttttctacagtttatcttttattttctgcaaatttaggaacatatttactcgttttcacattgaatcttaagtttaagctcttcatttggtatttaggcaatatatgagaaaaaaattttttttgttcatttgtaattttaacaagttgaacattttaccatgattgaacatgtttttattacagtatttaacattcccccaaagaataccctgcaaagtgtaaacctttgtcccatactgtgatattactgttctgctacaataaatgtcaaacctaa

SEQ ID NO: 8 - DNA, 4974 bp, Homo sapiens
acacctccctccccgcctgccagtgtcaccagcctgttgcctctgtgagaaagtaccactgtaagaggccaaagggcatgatcattttcctctttcaccctgtctaggttgccagcaaatcccacgggcctcctgacgctgcccctggggccacaggtccctcgagtgctggaaggatgaaggattcctgcatcactgtgatggccatggcgctgctgtctgggttctttttcttcgcgccggcctcgagctacaacctggacgtgcggggcgcgcggagcttctccccaccgcgcgccgggaggcactttggataccgcgtcctgcaggtcggaaacggggtcatcgtgggagctccaggggaggggaacagcacaggaagcctctatcagtgccagtcgggcacaggacactgcctgccagtcaccctgagaggttccaactatacctccaagtacttgggaatgaccttggcaacagaccccacagatggaagcattttgtttgctgctgttcagttttccacaagctacaaaacagaatttgatttctcagattatgttaaacggaaggaccctgatgctctgctgaagcatgtaaagcacatgttgctgttgaccaatacctttggtgccatcaattatgtcgcgacagaggtgttccgggaggagctgggggcccggccagatgccaccaaagtgcttatcatcatcacggatggggaggccactgacagtggcaacatcgatgcggccaaagacatcatccgctacatcatcgggattggaaagcattttcagaccaaggagagtcaggagaccctccacaaatttgcatcaaaacccgcgagcgagtttgtgaaaattctggacacatttgagaagctgaaagatctattcactgagctgcagaagaagatctatgtcattgagggcacaagcaaacaggacctgacttccttcaacatggagctgtcctccagcggcatcagtgctgacctcagcaggggccatgcagtcgtgggggcagtaggagccaaggactgggctgggggctttcttgacctgaaggcagacctgcaggatgacacatttattgggaatgaaccattgacaccagaagtgagagcaggctatttgggttacaccgtgacctggctgccctcccggcaaaagacttcgttgctggcctcgggagcccctcgataccagcacatgggccgagtgctgctgttccaagagccacagggcggaggacactggagccaggtccagacaatccatgggacccagattggctcttatttcggtggggagctgtgtggcgtcgacgtggaccaagatggggagacagagctgctgctgattggtgccccactgttctatggggagcagagaggaggccgggtgtttatctaccagagaagacagttggggtttgaagaagtctcagagctgcagggggaccccggctacccactcgggcggtttggagaagccatcactgctctgacagacatcaacggcgatgggctggtagacgtggctgtgggggcccctctggaggagcagggggctgtgtacatcttcaatgggaggcacggggggcttagtccccagccaagtcagcggatagaagggacccaagtgctctcaggaattcagtggtttggacgctccatccatggggtgaaggaccttgaaggggatggcttggcagatgtggctgtgggggctgagagccagatgatcgtgctgagctcccggcccgtggtggatatggtcaccctgatgtccttctctccagctgagatcccagtgcatgaagtggagtgctcctattcaaccagtaacaagatgaaagaaggagttaatatcacaatctgtttccagatcaagtctctcatcccccagttccaaggccgcctggttgccaatctcacttacactctgcagctggatggccaccggaccagaagacgggggttgttcccaggagggagacatgaactcagaaggaatatagctgtcaccaccagcatgtcatgcactgacttctcatttcatttcccggtatgtgttcaagacctcatctcccccatcaatgtttccctgaatttctctctttgggaggaggaagggacaccgagggaccaaagggcgggcaaggacataccgcccatcctgagaccctccctgcactcggaaacctgggagatcccttttgagaagaactgtggggaggacaagaagtgtgaggcaaacttgagagtgtccttctctcctgcaagatccagagccctgcgtctaactgcttttgccagcctctctgtggagctgagcctgagtaacttggaagaagatgcttactgggtccagctggacctgcacttccccccgggactctccttccgcaaggtggagatgctgaagccccatagccagatacctgtgagctgcgaggagcttcctgaagagtccaggcttctgtccagggcattatcttgcaatgtgagctctcccatcttcaaagcaggccactcggttgctctgcagatgatgtttaatacactggtaaacagctcctggggggactcggttgaattgcacgccaatgtgacctgtaacaatgaggactcagacctcctggaggacaactcagccactaccatcatccccatcctgtaccccatcaacatcctcatccaggaccaagaagactccacactctatgtcagtttcacccccaaaggccccaagatccaccaagtcaagcacatgtaccaggtgaggatccagccttccatccacgaccacaacatacccaccctggaggctgtggttggggtgccacagcctcccagcgaggggcccatcacacaccagtggagcgtgcagatggagcctcccgtgccctgccactatgaggatctggagaggctcccggatgcagctgagccttgtctccccggagccctgttccgctgccctgttgtcttcaggcaggagatcctcgtccaagtgatcgggactctggagctggtgggagagatcgaggcctcttccatgttcagcctctgcagctccctctccatctccttcaacagcagcaagcatttccacctctatggcagcaacgcctccctggcccaggttgtcatgaaggttgacgtggtgtatgagaagcagatgctctacctctacgtgctgagcggcatcggggggctgctgctgctgctgctcattttcatagtgctgtacaaggttggtttcttcaaacggaacctgaaggagaagatggaggctggcagaggtgtcccgaatggaatccctgcagaagactctgagcagctggcatctgggcaagaggctggggatcccggctgcctgaagcccctccatgagaaggactctgagagtggtggtggcaaggactgagtccaggcctgtgaggtgcagagtgcccagaactggactcaggatgcccagggccactctgcctctgcctgcattctgccgtgtgccctcgggcgagtcactgcctctccctggccctcagtttccctatctcgaacatggaactcattcctgcctgtctcctttgcaggctcatagggaagacctgctgagggaccagccaagagggctgcaaaagtgagggcttgtcattaccagacggttcaccagcctctcttggtttccttccttggaagagaatgtctgatctaaatgtggagaaactgtagtctcaggacctagggatgttctggccctcacccctgccctgggatgtccacagatgcctccaccccccagaacctgtccttgcacactcccctgcactggagtccagtctcttctgctggcagaaagcaaatgtgacctgtgtcactacgtgactgtggcacacgccttgttcttggccaaagaccaaattccttggcatgccttccagcaccctgcaaaatgagaccctcgtggccttccccagcctcttctagagccgtgatgcctccctgttgaagctctggtgacaccagcctttctcccaggccaggctccttcctgtcttcctgcattcacccagacagctccctctgcctgaaccttccatctcgccacccctccttccttgaccagcagatcccagctcacgtcacacttggttgggtcctcacatctttcacacttccaccagcctgcactactccctcaaagcacacgtcatgtttcttcatccggcagcctggatgttttttccctgtttaatgattgacgtacttagcagctatctctcagtgaactgtgagggtaaaggctatacttgtcttgttcaccttgggatgatgcctcatgatatgtcagggcgtgggacatctagtaggtgcttgacataatttcactgaattaatgacagagccagtgggaagatacagaaaaagaggggctgggctgggcgcggtggttcacgcctgtaatcccagcactttgggaggccaaggagggtggatcacctgaggtcaggagttagaggccagcctggcgaaaccccatctctactaaaaatacaaaatccaggcgtggtggcacacacctgtagtcccagctactcaggaggttgaggtaggagaattgcttgaacctgggaggtggaggttgcagtgagccaagattgcgccattgcactccagcctgggcaacacagcgagactccgtctcaaggaaaaaataaaaataaaaagcgggcacgggcccgtgacatccccacccttggaggctgtcttctcaggctctgccctgccctagctccacaccctctcccaggacccatcacgcctgtgcagtggcccccacagaaagactgagctcaaggtgggaaccacgtctgctaacttggagccccagtgccaagcacagtgcctgcatgtatttatccaataaatgtgaaattctgtccaaaaaaaaaaaaaaaaaa

SEQ ID NO: 9 - DNA, 2689 bp, Homo sapiens
tttattctctggaacatgaaacattctgttgtgctcatatcatgcaaattatcactagtaggagagcagagagtggaaatgttccaggtataaagacccacaagataaagaagctcagagtcgttagaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaagcctttttcacatagctcttggctgtaggattgccccactccaaaaaccagtgtgtggaggtccaggagtgagaccaggaaagaatgtgaaagtgactacacaaggactcctcgatggtcgtggaaaaggaaagtcaattggcagagcccctgaagccagtcttcaggacaaagaaggagcctagagacagaaatgacagatctctgctttggaaatcacacgtctggcttcacagatgtgtgattcacagtgtgaatcttggtgtctacgttaccaggcaggaaggctgagaggagagagactccagctgggttggaaaacagtattttccaaactaccttccagttcctcatttttgaatacaggcatagagttcagactttttttaaatagtaaaaataaaattaaagctgaaaactgcaacttgtaaatgtggtaaagagttagtttgagttactatcatgtcaaacgtgaaaatgctgtattagtcacagagataattctagctttgagcttaagaattttgagcaggtggtatgtttgggagactgctgagtcaacccaatagttgttgattggcaggagttggaagtgtgtgatctgtgggcacattagcctatgtgcatgcagcatctaagtaatgatgtcgtttgaatcacagtatacgctccatcgctgtcatctcagctggatctccattctctcaggcttgctgccaaaagccttttgtgttttgttttgtatcattatgaagtcatgcgtttaatcacattcgagtgtttcagtgcttcgcagatgtccttgatgctcatattgttccctattttgccagtgggaactcctaaatcaagttggcttctaatcaaagcttttaaaccctattggtaaagaatggaaggtggagaagctccctgaagtaagcaaagactttcctcttagtcgagccaagttaagaatgttcttatgttgcccagtgtgtttctgatctgatgcaagcaagaaacactgggcttctagaaccaggcaacttgggaactagactcccaagctggactatggctctactttcaggccacatggctaaagaaggtttcagaaagaagtggggacagagcagaactttcaccttcatatatttgtatgatcctaatgaatgcataaaatgttaagttgatggtgatgaaatgtaaatactgtttttaacaactatgatttggaaaataaatcaatgctataactatgttgaaaaaaaaaaaaaaaaaa

SEQ ID NO: 10 - DNA, 2335 bp, Homo sapiens
tttattctctggaacatgaaacattctgttgtgctcatatcatgcaaattatcactagtaggagagcagagagtggaaatgttccaggtataaagacccacaagataaagaagctcagagtcgttagaaacaggagcagatgtacagggtttgcctgactcacactcaaggttgcataagcaagatttcaaaattaatcctattctggagacctcaacccaatgtacaatgttcctgactggaaaagaagaactatatttttctgattttttttttcaaatctttaccattagttgccctgtatctccgccttcactttctgcaggaaactttatttcctacttctgcatgccaagtttctacctctagatctgtttggttcagttgctgagaagcctgacataccaggactgcctgagacaagccacaagctgaacagagaaagtggattgaacaaggacgcatttccccagtacatccacaacatgctgtccacatctcgttctcggtttatcagaaataccaacgagagcggtgaagaagtcaccaccttttttgattatgattacggtgctccctgtcataaatttgacgtgaagcaaattggggcccaactcctgcctccgctctactcgctggtgttcatctttggttttgtgggcaacatgctggtcgtcctcatcttaataaactgcaaaaagctgaagtgcttgactgacatttacctgctcaacctggccatctctgatctgctttttcttattactctcccattgtgggctcactctgctgcaaatgagtgggtctttgggaatgcaatgtgcaaattattcacagggctgtatcacatcggttattttggcggaatcttcttcatcatcctcctgacaatcgatagatacctggctattgtccatgctgtgtttgctttaaaagccaggacggtcacctttggggtggtgacaagtgtgatcacctggttggtggctgtgtttgcttctgtcccaggaatcatctttactaaatgccagaaagaagattctgtttatgtctgtggcccttattttccacgaggatggaataatttccacacaataatgaggaacattttggggctggtcctgccgctgctcatcatggtcatctgctactcgggaatcctgaaaaccctgcttcggtgtcgaaacgagaagaagaggcatagggcagtgagagtcatcttcaccatcatgattgtttactttctcttctggactccctataatattgtcattctcctgaacaccttccaggaattcttcggcctgagtaactgtgaaagcaccagtcaactggaccaagccacgcaggtgacagagactcttgggatgactcactgctgcatcaatcccatcatctatgccttcgttggggagaagttcagaaggtatctctcggtgttcttccgaaagcacatcaccaagcgcttctgcaaacaatgtccagttttctacagggagacagtggatggagtgacttcaacaaacacgccttccactggggagcaggaagtctcggctggtttataaaacgaggagcagtttgattgttgtttataaagggagataacaatctgtatataacaacaaacttcaagggtttgttgaacaatagaaacctgtaaagcaggtgcccaggaacctcagggctgtgtgtactaatacagactatgtcacccaatgcatatccaacatgtgctcagggaataatccagaaaaactgtgggtagagactttgactctccagaaagctcatctcagctcctgaaaaatgcctcattaccttgtgctaatcctctttttctagtcttcataatttcttcactcaatctctgattctgtcaatgtcttgaaatcaagggccagctggaggtgaagaagagaatgtgacaggcacagatgaatgggagtgagggatagtggggtcagggctgagaggagaaggagggagacatgagcatggctgagcctggacaaagacaaaggtgagcaaagggctcacgcattcagccaggagatgatactggtccttagccccatctgccacgtgtatttaaccttgaagggttcaccaggtcagggagagtttgggaactgcaataacctgggagttttggtggagtccgatgattctcttttgcataagtgcatgacatatttttgctttattacagtttatctatggcacccatgcaccttacatttgaaatctatgaaatatcatgctccattgttcagatgcttcttaggccacatccccctgtctaaaaattcagaaaatttttgtttataaaaga

SEQ ID NO: 11 - DNA, 7801 bp, Homo sapiens
atttgactcactgaccctgatggggaagatagtgttattcccacagtgtggtagtacacactggggcttatagggactgagcctactcaagggtatatggtgctgtgggtcagagctggggcatggcccagggattcagtgtgccttgactccccctgtaaatgttcctctcagaagccttcttggccttccagcccttggtttttgagacaaccagcagtcatttgttcgttcctgacattccttcctgtcccttccttccaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 12 - DNA, 7694 bp, Homo sapiens
atttgactcactgaccctgatggggaagatagtgttattcccacagtgtggtagtacacactggggcttatagggactgagcctactcaagggtatatggtgctgtgggtcagagctggggcatggcccagggattcagtgtgccttgactccccctgttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 13 - DNA, 7629 bp, Homo sapiens
atttgactcactgaccctgatggggaagatagtgttattcccacagtgtggtagtacacactggggcttatagggactgagcctactcaagggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 14 - DNA, 7573 bp, Homo sapiens
attatacttcttacagtgaggagccagagcttccaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 15 - DNA, 4809 bp, Homo sapiens
gtgaactgttgcaccgtgcaattgcacactataaatgtctttccttatctgtgtgtactcttatctcactgttctattttttctcctcatttatattaactctttcttacctttttttctgaacttctaggccttctctttccagaactggtggaagacaaatgaaacggccaagatggtaagaaacaagccgcatttctccttggggagactgataatttaaaaggtttgttgtgtcagaaacattcccagcttcatcaccaaccctttccttccacctctgcccactggagaccacttatatcccgaagcggacgcggcagctgaagtcaggaaaccatgcatcacattagcaggagccaactgcagactttaaactccgttcaacatgtggatgcggcagagaaatgacctgtccagacaagccggggcagctcataaactggttcatctgctccctgtgcgtcccgcgggtgcgtaagctctggagcagccggcgtccaaggacccggagaaaccttctgctgggcactgcgtgtgccatctacttgggcttcctggtgagccaggtggggagggcctctctccagcatggacaggcggctgagaaggggccacatcgcagccgcgacaccgccgagccatccttccctgagatacccctggatggtaccctggcccctccagagtcccagggcaatgggtccactctgcagcccaatgtggtgtacattaccctacgctccaagcgcagcaagccggccaatatccgtggcaccgtgaagcccaagcgcaggaaaaagcatgcagtggcatcggctgccccagggcaggaggctttggtcggaccatcccttcagccgcaggaagcggcaagggaagctgatgctgtagcacctgggtacgctcagggagcaaacctggttaagattggagagcgaccctggaggttggtgcggggtccgggagtgcgagccgggggcccagacttcctgcagcccagctccagggagagcaacattaggatctacagcgagagcgccccctcctggctgagcaaagatgacatccgaagaatgcgactcttggcggacagcgcagtggcagggctccggcctgtgtcctctaggagcggagcccgtttgctggtgctggaggggggcgcacctggcgctgtgctccgctgtggccctagcccctgtgggcttctcaagcagcccttggacatgagtgaggtgtttgccttccacctagacaggatcctggggctcaacaggaccctgccgtctgtgagcaggaaagcagagttcatccaagatggccgcccatgccccatcattctttgggatgcatctttatcttcagcaagtaatgacacccattcttctgttaagctcacctggggaacttatcagcagttgctgaaacagaaatgctggcagaatggccgagtacccaagcctgaatcgggttgtactgaaatacatcatcatgagtggtccaagatggcactctttgattttttgttacagatttataatcgcttagatacaaattgctgtggattcagacctcgcaaggaagatgcctgtgtacagaatggattgaggccaaaatgtgatgaccaaggttctgcggctctagcacacattatccagcgaaagcatgacccaaggcatttggtttttatagacaacaagggtttctttgacaggagtgaagataacttaaacttcaaattgttagaaggcatcaaagagtttccagcttctgcagtttctgttttgaagagccagcacttacggcagaaacttcttcagtctctgtttcttgataaagtgtattgggaaagtcaaggaggtagacaaggaattgaaaagcttatcgatgtaatagaacacagagccaaaattcttatcacctatatcaatgcacacggggtcaaagtattacctatgaatgaatgacaaaagaatcttctggctagggtgttagatatatttatgcatttttggttttgtttttaaatcaagcacatcaacctcaagcccgtttagcaatgaggcagtgtagatgaatacgtaaaataaatgactttaaccaagtagctataatgggacttagcactgtatgcatacttaaaaaggttttgaaaaacaaactacttgagaaatatttgtttatatttttctctaacatcatgctatgtgtcagtctgaacatctgacaacagaaatttcagttattattctagctaagttttgaaaacatttgtcatgctgtttaatagaaaactgcaaaccagagacactgactccattaataaaccatattttgtgccgttttgactgttctgaccaaatactaatgggaacaattcttgacgtttttctgttgctgattgttaacatagagcagtctctacactaccctgaggcaactctacattggaacactgaggcttacagcctgcaagagcatcagagctgaccatacatttaaacagaaatgctggtttatttgcaaaatcaccagtatattttctattgtgtctataaaaaatcagtcatttaagtacaagaatcatattttccattcctttttagaaatttattttgttgtccctatggaaatcattcacatctgacaatttatatgttaaagagttttactctctctattttggtccaatttgtatctagtggctgagaaattaaataattctaaagtatgaagttacctatctgaaaatgtacttacagagtatcattttaaaatggatgtctctttaaaaattttgttacttttaccaacaatgtaatataatttatgtatattttattaataatagtgaattccttaaaatttgttctatgtacttatatttaatttgatttaatggttactgcccagatattgagaattggttcaaatattgagtgtgtttcaatatattatctggcttatttcaacatgagtaatatgagcaaaataagttaaaacctgcgtctgatcaattttcctcatgactagaactaaaacagtaaatttggacaatattaagcctcaaataatcatctccaaactccttctaacactttttaaatcagattggaagacatggacaaatcaggttcatgtgttgcatctttatgtcctttgccaatatccaagatcatcacatatggtagatattcacatggagtttcaaattcagaatagattaccattaccttcctgcccttacacatcctactccttatttaaaagttctatttgtgacttttcatttcctgaaagtttaaaaatacaatttgagaatgtttataatacattctctcctgtcttttcacggttacgtctgttattgctgaaatacaccacattttctttgttctggtcaaggttaactcaatatctgtgtgaaagagaactactaacaacgttacaatagaggctagatttgaaaaaaaaaatctatagatctaattgatacaattgtagaacaaaatgtcaaaataatgttttaagtataagagaagatggaccaaggagagagagatcatttgaaaatctaattgtagcttttctaggctcacattcatgtactacttttagcacccttatgggctgtgctcgccccctggacagttgagctttggattatcttcctcttcaattttccctctattgacccgagtgtctccctctgcttctacagatttatagtactccttggctcttttgagtctccacttttactcactgtctctgggatttttaagatccttttcttctcttataaatcatcctcttaatgaaaattagcctaacaaaagtttggagactggaatcctactttgagccactgacttgaaataactcttttggcaagttgcctgacatcctgtcttaccaaggtggcatatttgcatttttactgcttaaaacatttttttttttttaccatctttatccaaatttatcatattgatggtaggactaacaggctttttagaagctggctttaactttgagtctcaagctacaatgctgttgggcagcctggtcttcccacgtgagggtttaactttgtttatttgcctccagttattccaaaatgcttattaaatgaaagtcccaggaacatgtttattttagtcacctttgctttttaacaattttgttttgtaatcaatgagtaattcatgatgaattatttttgactaatggatagccgaaggccaggcttttaattctaataggtaatgttcttcttttgtcttattgaaacaatgagaatactctgtgcatttcaaatgcactccgattatgctgtggttttattcacataagcacaatatgtgttttatttataacttcataacaaacttataatataataatttaccttagcagacatgcaaaagcttattcttgtgtgacttactttctttaagctaataatataaaaataaatatgtatcttaaaaatctataataaaacattagaaattaaagatatgtgctttttattttgcagatgagttcatttgcttttgtagatgtgttttcagagctaggtacagaggaatgtttgctacctttagcggtgaaaaaagaaagagagtcaagaattttgttggattgtgtttgtgtgtgcatatatttgatatcatcattatatttgtaatctttggacttgtaatcatagcctgtttattctactgtgccattaaatatactttaccttatacataacgaataaaatacctagaagtagatttatttacaaaaaaaaaaaaaaaa

SEQ ID NO: 16 - DNA, 3522 bp, Homo sapiens
aggtcggggtgagaggccgggggcctggtctgggccggagcctgccggggacaggaactggatgacgggccaggccagattccaccgcggagccgaggcatgccgagctatccggcagtgcaggtcccggcggccgcgggcagctcacccggaaggccgcgcggggtccgggcgtggatcccggcacgcgagcccgggcgttcgcaggggaggattttccttgctgctgcttaacttggaggagtactactttgaacagcatagagccaatcacattttgcacaagggcagtcaccatgaaaggaaaatcagaggctccttaaaaatatgttcaaaatcggtgatttttgaaccagattcaatatcccagcccatcatcaagattcctttgagagactgtataaaaataggaaagcatggagaaaatggagccaatagacacttcacaaaggcaaaatctgggggtatttcactcattttcagtcaggtatatttcattaaagaacataatgttgttgcaccatataaaatagaaaggggcaaaatggaatatgtttttgaattggatgttcccgggaaagtggaagatgttgtggagacgttgcttcagcttcacagagcatcctgccttgacaaattgggtgaccaaaccgccatgataacagctattttgcagtctcgtttagctagaacatcatttgacaaaaacaggttccaaaacatttctgaaaagctgcacatggaatgcaaagcagaaatggtgacgcctctggtgactaatcctggacacgtgtgcatcacggacacaaacctgtattttcagcccctcaacggctacccgaaacctgtggtccagataacactccaagatgtccgccgcatctacaaaaggaggcacggcctcatgcctctgggcttggaagtattttgcacagaagatgatctgtgttccgacatctacctaaagttctatgaacctcaagatagagatgatctctatttttacattgccacatacctagagcaccatgtggcggagcacactgctgagagctacatgctgcagtggcagcgtggacacctttccaactatcagtacctccttcacctcaacaacctggccgaccgcagctgcaacgacctctcccagtaccctgtgtttccatggataatacatgattattccagctcagaactagatttgtcaaatccaggaaccttccgggatctcagtaagccagtaggggccctaaataaggaacggctggagagactactgacacgctaccaggaaatgcctgaaccaaagttcatgtatgggagtcactactcttccccgggttatgtacttttttatcttgttaggattgcaccagagtatatgctgtgcctgcagaatggaagatttgataatgcagatagaatgttcaacagtattgcagaaacttggaaaaactgtctggatggtgcaacggattttaaagagttaattccagaattctatggtgatgatgtgagctttctagtcaatagcctgaagttggatttgggaaagagacaaggaggacagatggttgacgacgtggagcttcccccttgggcttccagtcccgaggactttctccagaagagcaaagatgcattggaaagcaattatgtgtctgaacaccttcacgagtggattgatctaatatttggctacaaacaaaaagggagtgatgcagttggggcccataatgtatttcatcccctgacctatgaaggaggtgtagacttgaacagcatccaggatcctgatgagaaggtagccatgcttacgcaaatcttggaatttgggcagacaccaaaacaactatttgtgacaccacatcctcgaaggatcaccccaaagtttaaaagtttgtcccagacctccagttataatgcttctatggcagattccccaggtgaagagtcttttgaagacctgaccgaagaaagcaaaacactggcctggaataacatcaccaaactgcagttacacgagcactataaaatccacaaagaagcagttactggaatcacggtctctcgcaatggatcttcagtattcacaacatcccaagattccaccttgaagatgttttctaaagaatcaaaaatgctacaaagaagtatatcattttcaaatatggctttatcgtcttgtttacttttaccaggagatgccactgtcataacttcttcatgggataataatgtctatttttattccatagcatttggaagacgccaggacacgttaatgggacatgatgatgctgttagtaagatctgttggcatgacaacaggctatattctgcatcgtgggactctacagtgaaggtgtggtctggtgttcctgcagagatgccaggcaccaaaagacaccactttgacttgctggccgagctggaacatgatgtcagtgtagatacaatcagtttaaatgctgcaagcacactgttagtttccggcaccaaagaaggcacagtgaatatttgggacctcacaacggccaccttaatgcaccagattccatgccattcagggattgtatgtgacactgcttttagcccagatagtcgccatgtcctcagcacaggaacagatggctgtcttaatgtcattgatgtgcagacaggaatgctcatctcctccatgacatcagatgagccccagaggtgctttgtctgggatggaaattccgttttatctggcagtcagtctggtgaactgctcgtttgggacctccttggagcaaaaatcagtgagagaatacagggccacacaggtgctgtgacatgtatatggatgaatgaacagtgtagcagtatcatcacaggaggggaagacagacaaattatattctggaaattgcagtattaagtgccttttcctctcctgaatattaaattgaactctatttaatgcatttttaaaccaaacttttaaacggactggtgaatgtgcaatgttagtaattagaagttttaccacatggaaaatttgtggttttaaactttctaaatcatggtgacttcattgaaagccattagttgctattctcttagggcagataaaatgcggctgtgttaggaaaaacatgttacactgtaaggcagatgatcgtccccgtatgatgattgtcagaagacaggactaagtagcagagaatagctaagagataaattgggctggggaaacttgtcagaaagcactgaacaattaagaaattttccaagaaaatgtgcagtattctctgctacttctgaatctgttttgtcttcctaatctatcacaattgccacccatcgggttttgggtgtgtgttttcatagcgtggttactttctataatgctgtacccagattctaagaacctggagaaggattagcagttcttagtaagtttactgtgtataggaacggtttgtatttcattacagctattcatcttttctacattaaaaatatttttctctaaagaaaaaaaaaaaaaaa

SEQ ID NO: 17 - DNA, 2914 bp, Homo sapiens
gtgctctgagtttttggtttctgtttcaccttgtgtctgagctggtctgaaggctggttgttcagactgagcttcctgcctgcctgtaccccgccaacagcttcagaagaaggtgactggtggctgcctgaggaataccagtgggcaagagaattagcatttctggagcatctgctgtctgagcagcccctgggtgcgtccactttctgggcacgtgaggttgggccttggccgcctgagcccttgagttggtcacttgaaccttgggaatattgagattatattctcctgccttttaaaaagatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacaggttccacttctgccgcatgagctgggctgaagcaaacagccagtgccagacacagtctgtacctttctggcggagggtcgatcatctattaataagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgatggtgctattttgtttgttttgttttgttttgtttttttgagacagaatctcgctctgtcgcccaggctggagtgcagtggcgtgatctcggctcaccgcaagctccgcctcccgggttcaggccattctcctgcctcagcctcccgagtagctgggactacaggggcccgccaccacacctggctaattttttaaaaatatttttagtagagacagggtttcactgtgttagccagggtggtcttgatctcctgacctcgtgatccacccacctcggcctcccaaagtgctgggattacaggcgtgagccaccgcgcctggccgatggtactatttagatataacactatgtttatttactaattttctagattttctactttattaattgttttgcacttttttataagagctaaagttaaataggatattaacaacaataacactgtctcctttctcttatgcttaaggctttgggaatgtttttagctggtggcaataaataccagacacgtacaaaatccagctatgaatatagagggcttatgattcagattgttatctatcaactataagcccactgttaatattctattaactttaattctctttcaaagctaaattccacactaccacattaaaaaaattagaaagtagccacgtatggtggctcatgtctataatcccagcactttgggaggttgaggtgggaggattgcttgaacccaagaggtcaaggctgcagtgagccatgttcacaccgctgcactcaagcttgggtgacagaacaagaccccgtctcaaaaaaaattttttttttaataaaacaaaatttgtttgaaatcttttaaaaattcaaatgatttttacaagttttaaataagctctccccaaacttgctttatgccttcttattgcttttatgatatatatatgcttggctaactatatttgctttttgctaacaatgctctggggtctttttatgcatttgcatttgctctttcatctctgcttggattattttaaatcattaggaattaagttatctttaaaatttaagtatcttttttcaaaaacattttttaatagaataaaatataatttgatcttattaaa

SEQ ID NO: 18 - DNA, 2859 bp, Homo sapiens
ctttccgcggcattctttgggcgtgagtcatgcaggtttgcagccagccccaaagggggtgtgtgcgcgagcagagcgctataaatacggcgcctcccagtgcccacaacgcggcgtcgccaggaggagcgcgcgggcacagggtgccgctgaccgaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgatcgcttggagggtcctctttttatgttgagttgctgcttcccggcatgccttcattttgctatggggggcaggcaggggggatggaaaataagtagaaacaaaaaagcagtggctaagatggtatagggactgtcataccagtgaagaataaaagggtgaagaataaaagggatatgatgacaaggttgatccacttcaagaattgcttgctttcaggaagagagatgtgtttcaacaagccaactaaaatatattgctgcaaatggaagcttttctgttctattataaaactgtcgatgtattctgaccaaggtgcgacaatctcctaaaggaatacactgaaagttaaggagaagaatcagtaagtgtaaggtgtacttggtattataatgcataattgatgttttcgttatgaaaacatttggtgcccagaagtccaaattatcagttttatttgtaagagctattgcttttgcagcggttttatttgtaaaagctgttgatttcgagttgtaagagctcagcatcccaggggcatcttcttgactgtggcatttcctgtccaccgccggtttatatgatcttcatacctttccctggaccacaggcgtttctcggcttttagtctgaaccatagctgggctgcagtaccctacgctgccagcaggtggccatgactacccgtggtaccaatctcagtcttaaagctcaggcttttcgttcattaacattctctgatagaattctggtcatcagatgtactgcaatggaacaaaactcatctggctgcatcccaggtgtgtagcaaagtccacatgtaaatttatagcttagaatattcttaagtcactgtcccttgtctctctttgaagttataaacaacaaacttaaagcttagcttatgtccaaggtaagtattttagcatggctgtcaaggaaattcagagtaaagtcagtgtgattcacttaatgatatacattaattagaattatggggtcagaggtatttgcttaagtgatcataattgtaaagtatatgtcacattgtcacattaatgtcaaaaaaaaaaaaaaaaaa

SEQ ID NO: 19 - DNA, 1809 bp, Homo sapiens
gtcttcctccctccctcccttcctcttactctcattcatttcatacacactggctcacacatctactctctctctctatctctctcagaatgacaattctaggtacaacttttggcatggttttttctttacttcaagtcgtttctggagaaagtggctatgctcaaaatggagacttggaagatgcagaactggatgactactcattctcatgctatagccagttggaagtgaatggatcgcagcactcactgacctgtgcttttgaggacccagatgtcaacatcaccaatctggaatttgaaatatgtggggccctcgtggaggtaaagtgcctgaatttcaggaaactacaagagatatatttcatcgagacaaagaaattcttactgattggaaagagcaatatatgtgtgaaggttggagaaaagagtctaacctgcaaaaaaatagacctaaccactatagttaaacctgaggctccttttgacctgagtgtcgtctatcgggaaggagccaatgactttgtggtgacatttaatacatcacacttgcaaaagaagtatgtaaaagttttaatgcacgatgtagcttaccgccaggaaaaggatgaaaacaaatggacgcatgtgaatttatccagcacaaagctgacactcctgcagagaaagctccaaccggcagcaatgtatgagattaaagttcgatccatccctgatcactattttaaaggcttctggagtgaatggagtccaagttattacttcagaactccagagatcaataatagctcaggggagatggatcctatcttactaaccatcagcattttgagttttttctctgtcgctctgttggtcatcttggcctgtgtgttatggaaaaaaaggattaagcctatcgtatggcccagtctccccgatcataagaagactctggaacatctttgtaagaaaccaagaaaaaatttaaatgtgagtttcaatcctgaaagtttcctggactgccagattcatagggtggatgacattcaagctagagatgaagtggaaggttttctgcaagatacgtttcctcagcaactagaagaatctgagaagcagaggcttggaggggatgtgcagagccccaactgcccatctgaggatgtagtcatcactccagaaagctttggaagagattcatccctcacatgcctggctgggaatgtcagtgcatgtgacgcccctattctctcctcttccaggtccctagactgcagggagagtggcaagaatgggcctcatgtgtaccaggacctcctgcttagccttgggactacaaacagcacgctgccccctccattttctctccaatctggaatcctgacattgaacccagttgctcagggtcagcccattcttacttccctgggatcaaatcaagaagaagcatatgtcaccatgtccagcttctaccaaaaccagtgaagtgtaagaaacccagactgaacttaccgtgagcgacaaagatgatttaaaagggaagtctagagttcctagtctccctcacagcacagagaagacaaaattagcaaaaccccactacacagtctgcaagattctgaaacattgctttgaccactcttcctgagttcagtggcactcaacatgagtcaagagcatcctgcttctaccatgtggatttggtcacaaggtttaaggtgacccaatgattcagctatttaaaaaaaaaagaggaaagaatgaaagagtaaaggaaatgattgaggagtgaggaaggcaggaagagagcatgagaggaaaaaaa

SEQ ID NO: 20 - DNA, 5226 bp, Homo sapiens
acacctccctccccgcctgccagtgtcaccagcctgttgcctctgtgagaaagtaccactgtaagaggccaaagggcatgatcattttcctctttcaccctgtctaggttgccagcaaatcccacgggcctcctgacgctgcccctggggccacaggtccctcgagtgctggaaggatgaaggattcctgcatcactgtgatggccatggcgctgctgtctgggttctttttcttcgcgccggcctcgagctacaacctggacgtgcggggcgcgcggagcttctccccaccgcgcgccgggaggcactttggataccgcgtcctgcaggtcggaaacggggtcatcgtgggagctccaggggaggggaacagcacaggaagcctctatcagtgccagtcgggcacaggacactgcctgccagtcaccctgagaggttccaactatacctccaagtacttgggaatgaccttggcaacagaccccacagatggaagcattttggcctgtgaccctgggctgtctcgaacgtgtgaccagaacacctatctgagtggcctgtgttacctcttccgccagaatctgcagggtcccatgctgcaggggcgccctggttttcaggaatgtatcaagggcaacgtagacctggtatttctgtttgatggttcgatgagcttgcagccagatgaatttcagaaaattctggacttcatgaaggatgtgatgaagaaactcagcaacacttcgtaccagtttgctgctgttcagttttccacaagctacaaaacagaatttgatttctcagattatgttaaacggaaggaccctgatgctctgctgaagcatgtaaagcacatgttgctgttgaccaatacctttggtgccatcaattatgtcgcgacagaggtgttccgggaggagctgggggcccggccagatgccaccaaagtgcttatcatcatcacggatggggaggccactgacagtggcaacatcgatgcggccaaagacatcatccgctacatcatcgggattggaaagcattttcagaccaaggagagtcaggagaccctccacaaatttgcatcaaaacccgcgagcgagtttgtgaaaattctggacacatttgagaagctgaaagatctattcactgagctgcagaagaagatctatgtcattgagggcacaagcaaacaggacctgacttccttcaacatggagctgtcctccagcggcatcagtgctgacctcagcaggggccatgcagtcgtgggggcagtaggagccaaggactgggctgggggctttcttgacctgaaggcagacctgcaggatgacacatttattgggaatgaaccattgacaccagaagtgagagcaggctatttgggttacaccgtgacctggctgccctcccggcaaaagacttcgttgctggcctcgggagcccctcgataccagcacatgggccgagtgctgctgttccaagagccacagggcggaggacactggagccaggtccagacaatccatgggacccagattggctcttatttcggtggggagctgtgtggcgtcgacgtggaccaagatggggagacagagctgctgctgattggtgccccactgttctatggggagcagagaggaggccgggtgtttatctaccagagaagacagttggggtttgaagaagtctcagagctgcagggggaccccggctacccactcgggcggtttggagaagccatcactgctctgacagacatcaacggcgatgggctggtagacgtggctgtgggggcccctctggaggagcagggggctgtgtacatcttcaatgggaggcacggggggcttagtccccagccaagtcagcggatagaagggacccaagtgctctcaggaattcagtggtttggacgctccatccatggggtgaaggaccttgaaggggatggcttggcagatgtggctgtgggggctgagagccagatgatcgtgctgagctcccggcccgtggtggatatggtcaccctgatgtccttctctccagctgagatcccagtgcatgaagtggagtgctcctattcaaccagtaacaagatgaaagaaggagttaatatcacaatctgtttccagatcaagtctctcatcccccagttccaaggccgcctggttgccaatctcacttacactctgcagctggatggccaccggaccagaagacgggggttgttcccaggagggagacatgaactcagaaggaatatagctgtcaccaccagcatgtcatgcactgacttctcatttcatttcccggtatgtgttcaagacctcatctcccccatcaatgtttccctgaatttctctctttgggaggaggaagggacaccgagggaccaaagggcgcagggcaaggacataccgcccatcctgagaccctccctgcactcggaaacctgggagatcccttttgagaagaactgtggggaggacaagaagtgtgaggcaaacttgagagtgtccttctctcctgcaagatccagagccctgcgtctaactgcttttgccagcctctctgtggagctgagcctgagtaacttggaagaagatgcttactgggtccagctggacctgcacttccccccgggactctccttccgcaaggtggagatgctgaagccccatagccagatacctgtgagctgcgaggagcttcctgaagagtccaggcttctgtccagggcattatcttgcaatgtgagctctcccatcttcaaagcaggccactcggttgctctgcagatgatgtttaatacactggtaaacagctcctggggggactcggttgaattgcacgccaatgtgacctgtaacaatgaggactcagacctcctggaggacaactcagccactaccatcatccccatcctgtaccccatcaacatcctcatccaggaccaagaagactccacactctatgtcagtttcacccccaaaggccccaagatccaccaagtcaagcacatgtaccaggtgaggatccagccttccatccacgaccacaacatacccaccctggaggctgtggttggggtgccacagcctcccagcgaggggcccatcacacaccagtggagcgtgcagatggagcctcccgtgccctgccactatgaggatctggagaggctcccggatgcagctgagccttgtctccccggagccctgttccgctgccctgttgtcttcaggcaggagatcctcgtccaagtgatcgggactctggagctggtgggagagatcgaggcctcttccatgttcagcctctgcagctccctctccatctccttcaacagcagcaagcatttccacctctatggcagcaacgcctccctggcccaggttgtcatgaaggttgacgtggtgtatgagaagcagatgctctacctctacgtgctgagcggcatcggggggctgctgctgctgctgctcattttcatagtgctgtacaaggttggtttcttcaaacggaacctgaaggagaagatggaggctggcagaggtgtcccgaatggaatccctgcagaagactctgagcagctggcatctgggcaagaggctggggatcccggctgcctgaagcccctccatgagaaggactctgagagtggtggtggcaaggactgagtccaggcctgtgaggtgcagagtgcccagaactggactcaggatgcccagggccactctgcctctgcctgcattctgccgtgtgccctcgggcgagtcactgcctctccctggccctcagtttccctatctcgaacatggaactcattcctgcctgtctcctttgcaggctcatagggaagacctgctgagggaccagccaagagggctgcaaaagtgagggcttgtcattaccagacggttcaccagcctctcttggtttccttccttggaagagaatgtctgatctaaatgtggagaaactgtagtctcaggacctagggatgttctggccctcacccctgccctgggatgtccacagatgcctccaccccccagaacctgtccttgcacactcccctgcactggagtccagtctcttctgctggcagaaagcaaatgtgacctgtgtcactacgtgactgtggcacacgccttgttcttggccaaagaccaaattccttggcatgccttccagcaccctgcaaaatgagaccctcgtggccttccccagcctcttctagagccgtgatgcctccctgttgaagctctggtgacaccagcctttctcccaggccaggctccttcctgtcttcctgcattcacccagacagctccctctgcctgaaccttccatctcgccacccctccttccttgaccagcagatcccagctcacgtcacacttggttgggtcctcacatctttcacacttccaccagcctgcactactccctcaaagcacacgtcatgtttcttcatccggcagcctggatgttttttccctgtttaatgattgacgtacttagcagctatctctcagtgaactgtgagggtaaaggctatacttgtcttgttcaccttgggatgatgcctcatgatatgtcagggcgtgggacatctagtaggtgcttgacataatttcactgaattaatgacagagccagtgggaagatacagaaaaagaggggctgggctgggcgcggtggttcacgcctgtaatcccagcactttgggaggccaaggagggtggatcacctgaggtcaggagttagaggccagcctggcgaaaccccatctctactaaaaatacaaaatccaggcgtggtggcacacacctgtagtcccagctactcaggaggttgaggtaggagaattgcttgaacctgggaggtggaggttgcagtgagccaagattgcgccattgcactccagcctgggcaacacagcgagactccgtctcaaggaaaaaataaaaataaaaagcgggcacgggcccgtgacatccccacccttggaggctgtcttctcaggctctgccctgccctagctccacaccctctcccaggacccatcacgcctgtgcagtggcccccacagaaagactgagctcaaggtgggaaccacgtctgctaacttggagccccagtgccaagcacagtgcctgcatgtatttatccaataaatgtgaaattctgtccaaaaaaaaaaaaaaaaaa

SEQ ID NO: 21 - DNA, 2684 bp, Homo sapiens
acgccgtagggggcgggccgttcgggcttggtttcctgggcgaccaccgctggctagtccgttagaggcgtgcgggcttcggaggcgtgcgggcttcgggtgccatggggactcctcccggcctgcagaccgactgcgaggcgctgctcagccgcttccaggagacggacagtgtacgcttcgaggacttcacggagctctggagaaacatgaagttcgggactatcttctgtggcagaatgagaaatttagaaaagaacatgtttacaaaagaagctttagctttggcttggcgatattttttacctccatacaccttccagatcagagttggtgctttgtatctgctatatggattatataatacccaactgtgtcaaccaaaacaaaagatcagagttgccctgaaggattgggatgaagttttaaaatttcagcaagatttagtaaatgcacagcattttgatgcagcttatatttttaggaagctacgactagacagagcatttcactttacagcaatgcccaaattgctgtcatataggatgaagaaaaaaattcaccgagctgaagttacagaagaatttaaggacccaagtgatcgtgtgatgaaacttatcacttctgatgtattagaggaaatgctgaatgttcatgatcattatcagaacatgaaacatgtaatttcagttgataagtccaagccagataaagccctcagcttgataaaggatgatttttttgacaatattaagaacatagttttggagcatcagcagtggcacaaagacagaaagaatccatccttaaagtcaaaaactaatgatggagaagaaaaaatggaaggaaattcacaagaaacggagagatgtgaaagggcagaatcattagcgaaaataaaatcaaaggccttttcagttgtcatacaggcatccaaatcaagaaggcatcgtcaagtcaaactcgactcttctgactctgattctgcatctggtcaagggcaagtcaaagcaactaggaaaaaagagaagaaagaaagattgaaaccagcaggaaggaagatgtctctcagaaacaaaggcaatgtgcagaatatacacaaggaagataaacctttaagtctgagtatgcctgtaattacagaagaagaagagaatgaaagtttgagtggaacagagttcactgcatccaagaagaggagaaaacactgaacaaagagcctggtgtagtttttaattttgagttttctgacagaagaaaagattgatattttgtgtattgaacaggaagactgccagtattaaaaaaatccttctgggaatctgtaggttatttcttggaaattgcaatacgtagttctagaataaaagtacaaaaaattagaataagaattctttaacattttctttaatgatttgcataaatggagataaaacttgtatttagtatgtaatagaaaaaattctgttattcgcagattgttactatttcctataaggttttgtgatactatactgtcctaatacagtctggtaatactattctattttatttaaaatattttttattgaaatattaatgtttattacatgcaaataactattttgtatctacagtcggataatggattttttattttgtatatttattctattttgtatattgttaagtgcaataaagtttttgccttgctttattttttaatacataaaacttacattctcataacgtgattgataacttaggaagttcacaatgtattttctacttctgcaattaaatattctttagtgcttgtttattattactaaatactaattaagtactaacaagtacttaaatactaatgtattaagtatttaagtactttctaataaaatctttaacaataataatgtaaatttcagaatgtgtctctggtacagaatagttgatattaacagaaaaaaaaaaatctgtagcttcatgaatatgccactctgttaatttcttgttccagacattttaatagagattgcttgagccatgttgtttgaattgctgccaatagcagaccatatccctatcatgttgttggctcaactgttttttttttttccctaatagagatggagtatcgctatgttgctcaggctggtcttgaactcctgggctcaagctatcctcctgcctcagcctcccaaagtactgggattataggtgtgagctactgtacccagccttaacctgtttcacagttgattatacttcatgctgttttccagcatggtattattaagggatttaaagtttgggttgcatgcctgtaatcccagcattttgggaggccgaggtgggcggatcacgaggtcaggagatcgagaccatcgtgactaacacagtgaaaccccgtctctaataaaaatacgaaaaattagccaggcgtggtggcgggcgcctgtaatcccagctactcgggaagctgaggcaggagaatggtgtgaacccagtgagccgagatcgtgccactgcactccagcctgggcaacagagtgagacttcgtctcaaaaaaaaaaaaaagtttgggttgaagatcaaattcgtgatatctctatatctaatctttaaaaatcagaatgctaatgctgacgcaaataaaattttcatttattagcaaaaaaaaaaaaaaaaaaaaaaaaaaaaa

SEQ ID NO: 22 - DNA, 3431 bp, Homo sapiens
ggttttcaggagcccgagcgagggcgccgcttttgcgtccgggaggagccaaccgtggcgcaggcggcgcggggaggcgtcccagagtctcactctgccgcccaggctggactgcagtgacacaatctcggctgactgcaaccactgcctccagggttcaagcgattctcttgcctcagcctcccaagtagctgggattacagattgatgttcatgttcctgacactactacaagattcatactcctgatgctactgacaacgtggcttctccacagtcaccaaaccagggatgctatactggacttccctactctcatctgctccagccccctgaccttatagttgcccagctttcctggcaattgactttgcccatcaatacacaggatttagcatccagggaagatgtcggagcctcagatgttaattttctaattgagaatgttggcgctgtccgaacctggagacaggaaaacaaaaagtcctttctcctgattcaccaaaaaataaaatactgactaccatcactgtgatgagattcctatagtctcaggaactgaagtctttaaacaaccagggaccctctgcccctagaataagaacatactagaagtcccttctgctaggacaacgaggatcatgggagaccacctggaccttctcctaggagtggtgctcatggccggtcctgtgtttggaattccttcctgctcctttgatggccgaatagccttttatcgtttctgcaacctcacccaggtcccccaggtcctcaacaccactgagaggctcctgctgagcttcaactatatcaggacagtcactgcttcatccttcccctttctggaacagctgcagctgctggagctcgggagccagtatacccccttgactattgacaaggaggccttcagaaacctgcccaaccttagaatcttggacctgggaagtagtaagatatacttcttgcatccagatgcttttcagggactgttccatctgtttgaacttagactgtatttctgtggtctctctgatgctgtattgaaagatggttatttcagaaatttaaaggctttaactcgcttggatctatccaaaaatcagattcgtagcctttaccttcatccttcatttgggaagttgaattccttaaagtccatagatttttcctccaaccaaatattccttgtatgtgaacatgagctcgagcccctacaagggaaaacgctctccttttttagcctcgcagctaatagcttgtatagcagagtctcagtggactggggaaaatgtatgaacccattcagaaacatggtgctggagatactagatgtttctggaaatggctggacagtggacatcacaggaaactttagcaatgccatcagcaaaagccaggccttctctttgattcttgcccaccacatcatgggtgccgggtttggcttccataacatcaaagatcctgaccagaacacatttgctggcctggccagaagttcagtgagacacctggatctttcacatgggtttgtcttctccctgaactcacgagtctttgagacactcaaggatttgaaggttctgaaccttgcctacaacaagataaataagattgcagatgaagcattttacggacttgacaacctccaagttctcaatttgtcatataaccttctgggggaactttacagttcgaatttctatggactacctaaggtagcctacattgatttgcaaaagaatcacattgcaataattcaagaccaaacattcaaattcctggaaaaattacagaccttggatctccgagacaatgctcttacaaccattcattttattccaagcatacccgatatcttcttgagtggcaataaactagtgactttgccaaagatcaaccttacagcgaacctcatccacttatcagaaaacaggctagaaaatctagatattctctactttctcctacgggtacctcatctccagattctcattttaaatcaaaatcgcttctcctcctgtagtggagatcaaaccccttcagagaatcccagcttagaacagcttttccttggagaaaatatgttgcaacttgcctgggaaactgagctctgttgggatgtttttgagggactttctcatcttcaagttctgtatttgaatcataactatcttaattcccttccaccaggagtatttagccatctgactgcattaaggggactaagcctcaactccaacaggctgacagttctttctcacaatgatttacctgctaatttagagatcctggacatatccaggaaccagctcctagctcctaatcctgatgtatttgtatcacttagtgtcttggatataactcataacaagttcatttgtgaatgtgaacttagcacttttatcaattggcttaatcacaccaatgtcactatagctgggcctcctgcagacatatattgtgtgtaccctgactcgttctctggggtttccctcttctctctttccacggaaggttgtgatgaagaggaagtcttaaagtccctaaagttctcccttttcattgtatgcactgtcactctgactctgttcctcatgaccatcctcacagtcacaaagttccggggcttctgttttatctgttataagacagcccagagactggtgttcaaggaccatccccagggcacagaacctgatatgtacaaatatgatgcctatttgtgcttcagcagcaaagacttcacatgggtgcagaatgctttgctcaaacacctggacactcaatacagtgaccaaaacagattcaacctgtgctttgaagaaagagactttgtcccaggagaaaaccgcattgccaatatccaggatgccatctggaacagtagaaagatcgtttgtcttgtgagcagacacttccttagagatggctggtgccttgaagccttcagttatgcccagggcaggtgcttatctgaccttaacagtgctctcatcatggtggtggttgggtccttgtcccagtaccagttgatgaaacatcaatccatcagaggctttgtacagaaacagcagtatttgaggtggcctgaggatctccaggatgttggctggtttcttcataaactctctcaacagatactaaagaaagaaaaagaaaagaagaaagacaataacattccgttgcaaactgtagcaaccatctcctaatcaaaggagcaatttccaacttatctcaagccacaaataactcttcactttgtatttgcaccaagttatcattttggggtcctctctggaggttttttttttctttttgctactatgaaaacaacataaatctctcaattttcgtatcaacaccatgttctgtctcactaacctccaaatggaaaataatagatctagaaaattgcaactgcc

SEQ ID NO: 23 - DNA, 3582 bp, Homo sapiens
gccggagtccccacgcgaggatgctgcggtgagcgcggccgggacgccgcgtcgggctctcggccgcccagcggcgccgcaggggaagccaggccggcagccgcgcgctccccagccggccaccaatcccgccctccccggctcctccggacctcctcggcaggcggcggcggggcctgcctgtgcgctctgcgcccgcccgcgcctaccctccatggcgtttatccggaagaagcagcaggagcagcagctgcagctctactccaaggagagattttccttgctgctgcttaacttggaggagtactactttgaacagcatagagccaatcacattttgcacaagggcagtcaccatgaaaggaaaatcagaggctccttaaaaatatgttcaaaatcggtgatttttgaaccagattcaatatcccagcccatcatcaagattcctttgagagactgtataaaaataggaaagcatggagaaaatggagccaatagacacttcacaaaggcaaaatctgggggtatttcactcattttcagtcaggtatatttcattaaagaacataatgttgttgcaccatataaaatagaaaggggcaaaatggaatatgtttttgaattggatgttcccgggaaagtggaagatgttgtggagacgttgcttcagcttcacagagcatcctgccttgacaaattgggtgaccaaaccgccatgataacagctattttgcagtctcgtttagctagaacatcatttgacaaaaacaggttccaaaacatttctgaaaagctgcacatggaatgcaaagcagaaatggtgacgcctctggtgactaatcctggacacgtgtgcatcacggacacaaacctgtattttcagcccctcaacggctacccgaaacctgtggtccagataacactccaagatgtccgccgcatctacaaaaggaggcacggcctcatgcctctgggcttggaagtattttgcacagaagatgatctgtgttccgacatctacctaaagttctatgaacctcaagatagagatgatctctatttttacattgccacatacctagagcaccatgtggcggagcacactgctgagagctacatgctgcagtggcagcgtggacacctttccaactatcagtacctccttcacctcaacaacctggccgaccgcagctgcaacgacctctcccagtaccctgtgtttccatggataatacatgattattccagctcagaactagatttgtcaaatccaggaaccttccgggatctcagtaagccagtaggggccctaaataaggaacggctggagagactactgacacgctaccaggaaatgcctgaaccaaagttcatgtatgggagtcactactcttccccgggttatgtacttttttatcttgttaggattgcaccagagtatatgctgtgcctgcagaatggaagatttgataatgcagatagaatgttcaacagtattgcagaaacttggaaaaactgtctggatggtgcaacggattttaaagagttaattccagaattctatggtgatgatgtgagctttctagtcaatagcctgaagttggatttgggaaagagacaaggaggacagatggttgacgacgtggagcttcccccttgggcttccagtcccgaggactttctccagaagagcaaagatgcattggaaagcaattatgtgtctgaacaccttcacgagtggattgatctaatatttggctacaaacaaaaagggagtgatgcagttggggcccataatgtatttcatcccctgacctatgaaggaggtgtagacttgaacagcatccaggatcctgatgagaaggtagccatgcttacgcaaatcttggaatttgggcagacaccaaaacaactatttgtgacaccacatcctcgaaggatcaccccaaagtttaaaagtttgtcccagacctccagttataatgcttctatggcagattccccaggtgaagagtcttttgaagacctgaccgaagaaagcaaaacactggcctggaataacatcaccaaactgcagttacacgagcactataaaatccacaaagaagcagttactggaatcacggtctctcgcaatggatcttcagtattcacaacatcccaagattccaccttgaagatgttttctaaagaatcaaaaatgctacaaagaagtatatcattttcaaatatggctttatcgtcttgtttacttttaccaggagatgccactgtcataacttcttcatgggataataatgtctatttttattccatagcatttggaagacgccaggacacgttaatgggacatgatgatgctgttagtaagatctgttggcatgacaacaggctatattctgcatcgtgggactctacagtgaaggtgtggtctggtgttcctgcagagatgccaggcaccaaaagacaccactttgacttgctggccgagctggaacatgatgtcagtgtagatacaatcagtttaaatgctgcaagcacactgttagtttccggcaccaaagaaggcacagtgaatatttgggacctcacaacggccaccttaatgcaccagattccatgccattcagggattgtatgtgacactgcttttagcccagatagtcgccatgtcctcagcacaggaacagatggctgtcttaatgtcattgatgtgcagacaggaatgctcatctcctccatgacatcagatgagccccagaggtgctttgtctgggatggaaattccgttttatctggcagtcagtctggtgaactgctcgtttgggacctccttggagcaaaaatcagtgagagaatacagggccacacaggtgctgtgacatgtatatggatgaatgaacagtgtagcagtatcatcacaggaggggaagacagacaaattatattctggaaattgcagtattaagtgccttttcctctcctgaatattaaattgaactctatttaatgcatttttaaaccaaacttttaaacggactggtgaatgtgcaatgttagtaattagaagttttaccacatggaaaatttgtggttttaaactttctaaatcatggtgacttcattgaaagccattagttgctattctcttagggcagataaaatgcggctgtgttaggaaaaacatgttacactgtaaggcagatgatcgtccccgtatgatgattgtcagaagacaggactaagtagcagagaatagctaagagataaattgggctggggaaacttgtcagaaagcactgaacaattaagaaattttccaagaaaatgtgcagtattctctgctacttctgaatctgttttgtcttcctaatctatcacaattgccacccatcgggttttgggtgtgtgttttcatagcgtggttactttctataatgctgtacccagattctaagaacctggagaaggattagcagttcttagtaagtttactgtgtataggaacggtttgtatttcattacagctattcatcttttctacattaaaaatatttttctctaaagaaaaaaaaaaaaaaa

SEQ ID NO: 24 - DNA, 4268 bp, Homo sapiens
gccctgcgcactccctgctggggtgagcagcactgtaaagatgaagctggctaactggtactggctgagctcagctgttcttgccacttacggttttttggttgtggcaaacaatgaaacagaggaaattaaagatgaaagagcaaaggatgtctgcccagtgagactagaaagcagagggaaatgcgaagaggcaggggagtgcccctaccaggtaagcctgccccccttgactattcagctcccgaagcaattcagcaggatcgaggaggtgttcaaagaagtccaaaacctcaaggaaatcgtaaatagtctaaagaaatcttgccaagactgcaagctgcaggctgatgacaacggagacccaggcagaaacggactgttgttacccagtacaggagccccgggagaggttggtgataacagagttagagaattagagagtgaggttaacaagctgtcctctgagctaaagaatgccaaagaggagatcaatgtacttcatggtcgcctggagaagctgaatcttgtaaatatgaacaacatagaaaattatgttgacagcaaagtggcaaatctaacatttgttgtcaatagtttggatggcaaatgttcaaagtgtcccagccaagaacaaatacagtcacgtccagttcaacatctaatatataaagattgctctgactactacgcaataggcaaaagaagcagtgagacctacagagttacacctgatcccaaaaatagtagctttgaagtttactgtgacatggagaccatggggggaggctggacagtgctgcaggcacgtctcgatgggagcaccaacttcaccagaacatggcaagactacaaagcaggctttggaaacctcagaagggaattttggctggggaacgataaaattcatcttctgaccaagagtaaggaaatgattctgagaatagatcttgaagactttaatggtgtcgaactatatgccttgtatgatcagttttatgtggctaatgagtttctcaaatatcgtttacacgttggtaactataatggcacagctggagatgcattacgtttcaacaaacattacaaccacgatctgaagtttttcaccactccagataaagacaatgatcgatatccttctgggaactgtgggctgtactacagttcaggctggtggtttgatgcatgtctttctgcaaacttaaatggcaaatattatcaccaaaaatacagaggtgtccgtaatgggattttctggggtacctggcctggtgtaagtgaggcacaccctggtggctacaagtcctccttcaaagaggctaagatgatgatcagacccaagcactttaagccataaatcactctgttcattcctccaggtattcgttatctaatagggcaattaattccttcagcactttagaatatgccttgtttcatatttttcatagctaaaaaatgatgtctgacggctaggttcttatgctacacagcatttgaaataaagctgaaaaacaatgcattttaaaggagtcctttgttgttatgctgttatccaatgaacacttgcaagcaattagcaatattgagaattatacattagatttacaattcttttaatttctattgaaactttttctattgcttgtattacttgctgtatttaaaaaataattgttggctgggtgtggtagctcacgcctgtaatcccagcactttggaatgtcaaggcaggcagatcacttgaggtcaggagtttgagaccagcctggccaaacatgtgaaacgctgtctctattaaaaatacaaaaattagccgggcatggtggtacatgcctgtaatcctagctacttgggaggctgaggcaggagaatcgcttgaacctgagaggaagaggttgcagtgagccaagactgagccactgcactccagcatgggtgacagagaaaactctgtctcaaacaaaaaaataataaaatttattcagtaggctggattctacacaaagtaatctgtatttgggccatgatttaagcacatctgaaggtatatcactcttttcaggctataattatttgggtaatcttcattctgagacaaacttaatctatatcatttactttgcaacagaacaaccctacagcattttggttcccagactaagggaactaatatctatataattaaacttgttcatttatcattcatgaaatataaaatacttgtcatttaaaccgtttaaaaatgtggtagcataatgtcaccccaaaaagcattcagaaagcaatgtaactgtgaagaccagggtttaaaggtaattcatttatagtttataactccttagatgtttgatgttgaaaactgctttaacatgaaaattatcttcctctgctctgtgtgaacaatagcttttaatttaagattgctcactactgtactagactactggtaggtttttttggggggggtgggtagggatatgtgggtaatgaagcatttacttacaggctatcatactctgaggccaattttatctccaaagcaataatatcattaagtgattcacttcatagaaggctaagtttctctaggacagatagaaaacatgaattttgaaatatatagaacagtagttaaaatactatatatttcaaccctggctggtagattgcttattttactatcagaaactaaaagatagatttttacccaaacagaagtatctgtaatttttataattcatcaattctggaatgctatatataatatttaaaagactttttaaatgtgtttaatttcatcatcgtaaaaagggatcatctcagagagaacagcagtattctgcgtatttttaaaaatgctctagagtaacatttgaagtaattcactgtagtgtatgccagtcctagaaataatttttttaatttctggtgtctgtttctaatacactaaccaagttttcaaaatatatttacaaagatgcatctttacccattattttaaaatgattaaggaggatagttgcttcaggtaacaagctaatttttcaaatattaggcccttacagaactatttagtcaaaaagtaagatattcctttaaaatatataacccaaagctttcagttaaacatgatatatcacaaatactattaaaatgttaaagagaaatgcaaatagcattaaatgatgaccaaaatgtaaaatattgtagatttcaaaagctgtgtctctattaggtgggataccaaatgtaaatgatgtaactgacgttgttttttactttttactttttaaaaaagactaaaaacgttttgatattatacaatgtatttgtttcagataaggtcattgtcatttagtatatataattaatatatgtacaagtttaagtaaattcctgtgagtaaaaatggacttatcacaaaacatagttctaaagaaaggtatatgctcatatacacggtgtccattaatttaatgggaactaggtataacttcaggagaatttggcaaataattcattaatccatgtaaatattcaaaagcttgttctatccacattatttcaagggatcactttatttttcattatactttcacagcacttttctagtaaattctgtaacacagaaattccattttggaatcatttcatgttaccaataattctagacttttataacatttaacatgttgatggaaatagattacatctgctagaaccttttgccttaactattcaccaatatatgctaatattcataaatatggattgactgtttacaaacattagaatcttgtcttggttccattttgatggctaatatttgttatcttaattaagactatttctgaggtcatgattacttgaaaatattgactaaaactgggtccttagaaattccaggtggagctgatttacctatgactgaggggaaaaaaaaatcaaattttactgataatagtaatgctccaaatgaattaatgacacatctgttcaataaataaagagcttaaatatacaaaacataagaaatctgggcaacaaaacttgtggtctttacttttgaatagctacccaagaaaaggttttaaaggtaaaagttatgagtaatgtcatcacaataagctcttgtttaaaattcttttcttttatgtataattaggtttatgtttcatgtctttttaaaaccttataaaagatttaattatcacatctattcttcaatgtggaaatattaaatattgttggttgtaaaataa

SEQ ID NO: 25 - DNA, 4854 bp, Homo sapiens
gtttttaaaagctttgtatctcttaaaaccatgcagcagtcagtttccaagttttgctttgcaatcagtagttttcaagggagcttttaaagctgaactgaaatgtttgaaatgtggaacactcttgaccatgaaatatgttctacttacatgcctcagcctttaaaagttctttgcattagagtcaaggattacattcttcctggagccaagcatggggccagctgtaaacaagccgcatttctccttggggagactgataatttaaaaggtttgttgtgtcagaaacattcccagcttcatcaccaaccctttccttccacctctgcccactggagaccacttatatcccgaagcggacgcggcagctgaagtcaggaaaccatgcatcacattagcaggagccaactgcagactttaaactccgttcaacatgtggatgcggcagagaaatgacctgtccagacaagccggggcagctcataaactggttcatctgctccctgtgcgtcccgcgggtgcgtaagctctggagcagccggcgtccaaggacccggagaaaccttctgctgggcactgcgtgtgccatctacttgggcttcctggtgagccaggtggggagggcctctctccagcatggacaggcggctgagaaggggccacatcgcagccgcgacaccgccgagccatccttccctgagatacccctggatggtaccctggcccctccagagtcccagggcaatgggtccactctgcagcccaatgtggtgtacattaccctacgctccaagcgcagcaagccggccaatatccgtggcaccgtgaagcccaagcgcaggaaaaagcatgcagtggcatcggctgccccagggcaggaggctttggtcggaccatcccttcagccgcaggaagcggcaagggaagctgatgctgtagcacctgggtacgctcagggagcaaacctggttaagattggagagcgaccctggaggttggtgcggggtccgggagtgcgagccgggggcccagacttcctgcagcccagctccagggagagcaacattaggatctacagcgagagcgccccctcctggctgagcaaagatgacatccgaagaatgcgactcttggcggacagcgcagtggcagggctccggcctgtgtcctctaggagcggagcccgtttgctggtgctggaggggggcgcacctggcgctgtgctccgctgtggccctagcccctgtgggcttctcaagcagcccttggacatgagtgaggtgtttgccttccacctagacaggatcctggggctcaacaggaccctgccgtctgtgagcaggaaagcagagttcatccaagatggccgcccatgccccatcattctttgggatgcatctttatcttcagcaagtaatgacacccattcttctgttaagctcacctggggaacttatcagcagttgctgaaacagaaatgctggcagaatggccgagtacccaagcctgaatcgggttgtactgaaatacatcatcatgagtggtccaagatggcactctttgattttttgttacagatttataatcgcttagatacaaattgctgtggattcagacctcgcaaggaagatgcctgtgtacagaatggattgaggccaaaatgtgatgaccaaggttctgcggctctagcacacattatccagcgaaagcatgacccaaggcatttggtttttatagacaacaagggtttctttgacaggagtgaagataacttaaacttcaaattgttagaaggcatcaaagagtttccagcttctgcagtttctgttttgaagagccagcacttacggcagaaacttcttcagtctctgtttcttgataaagtgtattgggaaagtcaaggaggtagacaaggaattgaaaagcttatcgatgtaatagaacacagagccaaaattcttatcacctatatcaatgcacacggggtcaaagtattacctatgaatgaatgacaaaagaatcttctggctagggtgttagatatatttatgcatttttggttttgtttttaaatcaagcacatcaacctcaagcccgtttagcaatgaggcagtgtagatgaatacgtaaaataaatgactttaaccaagtagctataatgggacttagcactgtatgcatacttaaaaaggttttgaaaaacaaactacttgagaaatatttgtttatatttttctctaacatcatgctatgtgtcagtctgaacatctgacaacagaaatttcagttattattctagctaagttttgaaaacatttgtcatgctgtttaatagaaaactgcaaaccagagacactgactccattaataaaccatattttgtgccgttttgactgttctgaccaaatactaatgggaacaattcttgacgtttttctgttgctgattgttaacatagagcagtctctacactaccctgaggcaactctacattggaacactgaggcttacagcctgcaagagcatcagagctgaccatacatttaaacagaaatgctggtttatttgcaaaatcaccagtatattttctattgtgtctataaaaaatcagtcatttaagtacaagaatcatattttccattcctttttagaaatttattttgttgtccctatggaaatcattcacatctgacaatttatatgttaaagagttttactctctctattttggtccaatttgtatctagtggctgagaaattaaataattctaaagtatgaagttacctatctgaaaatgtacttacagagtatcattttaaaatggatgtctctttaaaaattttgttacttttaccaacaatgtaatataatttatgtatattttattaataatagtgaattccttaaaatttgttctatgtacttatatttaatttgatttaatggttactgcccagatattgagaattggttcaaatattgagtgtgtttcaatatattatctggcttatttcaacatgagtaatatgagcaaaataagttaaaacctgcgtctgatcaattttcctcatgactagaactaaaacagtaaatttggacaatattaagcctcaaataatcatctccaaactccttctaacactttttaaatcagattggaagacatggacaaatcaggttcatgtgttgcatctttatgtcctttgccaatatccaagatcatcacatatggtagatattcacatggagtttcaaattcagaatagattaccattaccttcctgcccttacacatcctactccttatttaaaagttctatttgtgacttttcatttcctgaaagtttaaaaatacaatttgagaatgtttataatacattctctcctgtcttttcacggttacgtctgttattgctgaaatacaccacattttctttgttctggtcaaggttaactcaatatctgtgtgaaagagaactactaacaacgttacaatagaggctagatttgaaaaaaaaaatctatagatctaattgatacaattgtagaacaaaatgtcaaaataatgttttaagtataagagaagatggaccaaggagagagagatcatttgaaaatctaattgtagcttttctaggctcacattcatgtactacttttagcacccttatgggctgtgctcgccccctggacagttgagctttggattatcttcctcttcaattttccctctattgacccgagtgtctccctctgcttctacagatttatagtactccttggctcttttgagtctccacttttactcactgtctctgggatttttaagatccttttcttctcttataaatcatcctcttaatgaaaattagcctaacaaaagtttggagactggaatcctactttgagccactgacttgaaataactcttttggcaagttgcctgacatcctgtcttaccaaggtggcatatttgcatttttactgcttaaaacatttttttttttttaccatctttatccaaatttatcatattgatggtaggactaacaggctttttagaagctggctttaactttgagtctcaagctacaatgctgttgggcagcctggtcttcccacgtgagggtttaactttgtttatttgcctccagttattccaaaatgcttattaaatgaaagtcccaggaacatgtttattttagtcacctttgctttttaacaattttgttttgtaatcaatgagtaattcatgatgaattatttttgactaatggatagccgaaggccaggcttttaattctaataggtaatgttcttcttttgtcttattgaaacaatgagaatactctgtgcatttcaaatgcactccgattatgctgtggttttattcacataagcacaatatgtgttttatttataacttcataacaaacttataatataataatttaccttagcagacatgcaaaagcttattcttgtgtgacttactttctttaagctaataatataaaaataaatatgtatcttaaaaatctataataaaacattagaaattaaagatatgtgctttttattttgcagatgagttcatttgcttttgtagatgtgttttcagagctaggtacagaggaatgtttgctacctttagcggtgaaaaaagaaagagagtcaagaattttgttggattgtgtttgtgtgtgcatatatttgatatcatcattatatttgtaatctttggacttgtaatcatagcctgtttattctactgtgccattaaatatactttaccttatacataacgaataaaatacctagaagtagatttatttacaaaaaaaaaaaaaaaa

SEQ ID NO: 26 - DNA, 1779 bp, Homo sapiens
gtgactgggtggggctgcctcacttctgcctgatttgggaagcgctgcaaggacaaccggctggggtccttgcgcgccgcggctcagggaggagcaccgactgcgccgcgtaagtgccgcctgccctgcgtgggtcgtgccagctcagcgggacaggtcctcgcctcggtccctcggacttagggagcgcggggcagaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaa

SEQ ID NO: 27 - DNA, 2913 bp, Homo sapiens
gaatggctggggttggctgaagagcgcacagtggagttttaatgtccgccatgttggccatggcgtattgaagagagcgagcgagagaggagcggagcggcacagcctcccaccctcccggctggtgttagtgcccggacggcgggctctgcgctccgcccctcaagtccccggcagcggttggcgagtgggggccgaacccccggttctccatgatcccgctggccggggccgtttccccagagcggagaggtatctgctgcgcctgagatgagtaaactgtcgtttcgggcgcgggcgctagacgcctcgaagccgctgccggttttccgctgtgaggatctgcccgacctgcacgaatacgcctcgataaacagggccgtgccgcagatgcccaccggaatggagaaggaagaggagtcggaacatcatcttcagcgggctatttcagcacagcaggtgtatggcgagaagagggataatatggttataccggtcccagaggcagaaagtaatattgcttactatgagtctatatatcctggggaatttaagatgccaaagcagctcattcacatacagccttttagtttggatgctgaacagcctgattatgatttggattctgaagatgaagtatttgtgaataaactgaaaaagaaaatggacatctgcccattgcaatttgaggagatgattgaccgcctagaaaaaggcagtggtcagcagccagtcagtctgcaggaagccaaactactgctaaaagaagatgatgaactaattagagaagtttatgaatattggattaaaaagagaaaaaactgtcgagggccatctcttattccatcagtaaaacaagagaagcgagatggttccagcacaaatgatccttatgtggcttttagaaggcgtactgaaaaaatgcagactcgaaaaaatcgcaaaaatgatgaagcctcttacgaaaaaatgcttaagctgcgacgagatctaagtcgagctgttactattctagagatgataaaaagaagagaaaaaagtaaaagagagctattgcacttaacactggaaattatggaaaagaggtataatttgggcgactacaatggagagatcatgtctgaggttatggcacagagacagccaatgaaacctacttatgccatccccatcatccctattactaatagcagtcaatttaaacaccaggaagcaatggatgtgaaggagttcaaagttaataagcaagataaagccgatcttatccgaccgaaacggaaatatgaaaagaagcccaaagtcttaccatcgtctgccgctgctactccccaacagacgagtcctgctgcactgccagtcttcaatgctaaagatctgaatcagtatgactttcccagctcagacgaagaacctctctcccaggttttgtctggctcttcggaagctgaggaagacaatgatcctgatggtccttttgctttccgtaggaaagcaggctgtcagtactatgctcctcacttagaccaaactggcaactggccttggactagtcctaaagatggaggattaggggatgtgcgatatagatactgcttaactactctcaccgtaccccaaaggtgtattggatttgcacgaagacgggttgggcgcggtggaagggtcttactggacagagctcattcagactatgacagtgtgtttcaccatctggatttggaaatgctttcctcaccacaacattctccagtcaatcagtttgccaatacctcagaaacaaatacctcggacaaatctttctctaaagacctcagtcagatactagtcaatatcaaatcatgtagatggcggcattttaggcctcggacaccatccctacatgacagtgacaatgatgaactctcctgtagaaaattatataggagtataaaccgaacaggaacagcacaacctgggacccagacatgcagtacctctacgcaaagtaaaagtagcagtggttcagcacactttgcatttacagccgaacaataccagcaacatcaacagcaactggcactcatgcagaaacagcagcttgcacaaattcagcaacagcaagcaaatagtaattcctccaccaacacatcacagaaccttgcatctaaccagcagaaaagtggctttcgcctgaatatacagggtttagaaagaacactacagggttttgtttctaagactttggattctgctagtgcacagtttgctgcttctgctttggtgacatcagaacaactgatgggattcaagatgaaggatgatgtggtgcttggaatcggggtgaatggcgtccttccagcctcaggagtatacaagggcttacacctcagtagtactacaccaacagcacttgtacatacaagtccatcaacggcaggttcagctttgttacagccttcaaatattacacagacttcaagttcccacagtgcactgagtcatcaagtaactgctgccaattctgcaacaactcaggttctgattgggaacaacattcgattaactgtaccttcatcagttgccactgtaaactctattgccccaataaatgcacgacatatacctaggactttaagtgctgttccatcatctgccttaaagctggccgctgcagcaaactgtcaagtttccaaggtcccatcttcatcctctgtagattcagttccaagggaaaatcatgaatcagaaaagccagcactgaacaacatagcagacaacacagtagcgatggaggtgacgtagcttcctccgagtggaactgcagcctggggacttgattaggtgctatgcatcagtagcattttgaatgcaagggatgatggaaagcagcacatgcgtttctgtggcagctgtggggacttgacagcaatgctc

SEQ ID NO: 28 - DNA, 1691 bp, Homo sapiens
gtgactgggtggggctgcctcacttctgcctgatttgggaagcgctgcaaggacaaccggctggggtccttgcgcgccgcggctcagggaggagcaccgactgcgccgcaccctgagagatggttggtgccatgtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatccaaaaaggaagagaattgagtttggtcggccctttcccaggactgaacatgaagagttatgccggcttcctcaccgtgaataagacttacaacagcaacctcttcttctggttcttcccagctcagatacagccagaagatgccccagtagttctctggctacagggtgggccgggaggttcatccatgtttggactctttgtggaacatgggccttatgttgtcacaagtaacatgaccttgcgtgacagagacttcccctggaccacaacgctctccatgctttacattgacaatccagtgggcacaggcttcagttttactgatgatacccacggatatgcagtcaatgaggacgatgtagcacgggatttatacagtgcactaattcagtttttccagatatttcctgaatataaaaataatgacttttatgtcactggggagtcttatgcagggaaatatgtgccagccattgcacacctcatccattccctcaaccctgtgagagaggtgaagatcaacctgaacggaattgctattggagatggatattctgatcccgaatcaattatagggggctatgcagaattcctgtaccaaattggcttgttggatgagaagcaaaaaaagtacttccagaagcagtgccatgaatgcatagaacacatcaggaagcagaactggtttgaggcctttgaaatactggataaactactagatggcgacttaacaagtgatccttcttacttccagaatgttacaggatgtagtaattactataactttttgcggtgcacggaacctgaggatcagctttactatgtgaaatttttgtcactcccagaggtgagacaagccatccacgtggggaatcagacttttaatgatggaactatagttgaaaagtacttgcgagaagatacagtacagtcagttaagccatggttaactgaaatcatgaataattataaggttctgatctacaatggccaactggacatcatcgtggcagctgccctgacagagcgctccttgatgggcatggactggaaaggatcccaggaatacaagaaggcagaaaaaaaagtttggaagatctttaaatctgacagtgaagtggctggttacatccggcaagcgggtgacttccatcaggtaattattcgaggtggaggacatattttaccctatgaccagcctctgagagcttttgacatgattaatcgattcatttatggaaaaggatgggatccttatgttggataaactaccttcccaaaagagaacatcagaggttttcattgctgaaaagaaaatcgtaaaaacagaaaatgtcataggaataaaaaaattatcttttcatatctgcaagatttttttcatcaataaaaattatccttgaaacaa

SEQ ID NO: 29 - DNA, 2769 bp, Homo sapiens
gtgctctgagtttttggtttctgtttcaccttgtgtctgagctggtctgaaggctggttgttcagactgagcttcctgcctgcctgtaccccgccaacagcttcagaagaaggagcagcccctgggtgcgtccactttctgggcacgtgaggttgggccttggccgcctgagcccttgagttggtcacttgaaccttgggaatattgagatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaagagagaagcagcagccttgaaggaagtcctgatgaattttcaaatggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgatggtgctattttgtttgttttgttttgttttgtttttttgagacagaatctcgctctgtcgcccaggctggagtgcagtggcgtgatctcggctcaccgcaagctccgcctcccgggttcaggccattctcctgcctcagcctcccgagtagctgggactacaggggcccgccaccacacctggctaattttttaaaaatatttttagtagagacagggtttcactgtgttagccagggtggtcttgatctcctgacctcgtgatccacccacctcggcctcccaaagtgctgggattacaggcgtgagccaccgcgcctggccgatggtactatttagatataacactatgtttatttactaattttctagattttctactttattaattgttttgcacttttttataagagctaaagttaaataggatattaacaacaataacactgtctcctttctcttatgcttaaggctttgggaatgtttttagctggtggcaataaataccagacacgtacaaaatccagctatgaatatagagggcttatgattcagattgttatctatcaactataagcccactgttaatattctattaactttaattctctttcaaagctaaattccacactaccacattaaaaaaattagaaagtagccacgtatggtggctcatgtctataatcccagcactttgggaggttgaggtgggaggattgcttgaacccaagaggtcaaggctgcagtgagccatgttcacaccgctgcactcaagcttgggtgacagaacaagaccccgtctcaaaaaaaattttttttttaataaaacaaaatttgtttgaaatcttttaaaaattcaaatgatttttacaagttttaaataagctctccccaaacttgctttatgccttcttattgcttttatgatatatatatgcttggctaactatatttgctttttgctaacaatgctctggggtctttttatgcatttgcatttgctctttcatctctgcttggattattttaaatcattaggaattaagttatctttaaaatttaagtatcttttttcaaaaacattttttaatagaataaaatataatttgatcttattaaa

SEQ ID NO: 30 - DNA, 2655 bp, Homo sapiens
gtagagctggctggctgtgcccttccgtccttcattagacgtttgctcttgtcttagatgctcagatggtagtggataggcctgtgacgaaggtgctaccatcgtgagagtaagattatattctcctgccttttaaaaagatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaaggggaggagttgtgtggggtaatgacaatctcggactctccaagagaacaggatagtgaatcacagactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcaggggctttgaccacgacctttgaagagcttcattttgagatcaagccccacgatgactgcacagtagagcaaatctatgagattttgaaaatctaccaactcatggaccacagtaacatggactgcttcatctgctgtatcctctcccatggagacaagggcatcatctatggcactgatggacaggaggcccccatctatgagctgacatctcagttcactggtttgaagtgcccttcccttgctggaaaacccaaagtgttttttattcaggcttgtcagggggataactaccagaaaggtatacctgttgagactgattcagaggagcaaccctatttagaaatggatttatcatcacctcaaacgagatatatcccggatgaggctgactttctgctggggatggccactgtgaataactgtgtttcctaccgaaaccctgcagagggaacctggtacatccagtcactttgccagagcctgagagagcgatgtcctcgaggcgatgatattctcaccatcctgactgaagtgaactatgaagtaagcaacaaggatgacaagaaaaacatggggaaacagatgcctcagcctactttcacactaagaaaaaaacttgtcttcccttctgattgatggtgctattttgtttgttttgttttgttttgtttttttgagacagaatctcgctctgtcgcccaggctggagtgcagtggcgtgatctcggctcaccgcaagctccgcctcccgggttcaggccattctcctgcctcagcctcccgagtagctgggactacaggggcccgccaccacacctggctaattttttaaaaatatttttagtagagacagggtttcactgtgttagccagggtggtcttgatctcctgacctcgtgatccacccacctcggcctcccaaagtgctgggattacaggcgtgagccaccgcgcctggccgatggtactatttagatataacactatgtttatttactaattttctagattttctactttattaattgttttgcacttttttataagagctaaagttaaataggatattaacaacaataacactgtctcctttctcttatgcttaaggctttgggaatgtttttagctggtggcaataaataccagacacgtacaaaatccagctatgaatatagagggcttatgattcagattgttatctatcaactataagcccactgttaatattctattaactttaattctctttcaaagctaaattccacactaccacattaaaaaaattagaaagtagccacgtatggtggctcatgtctataatcccagcactttgggaggttgaggtgggaggattgcttgaacccaagaggtcaaggctgcagtgagccatgttcacaccgctgcactcaagcttgggtgacagaacaagaccccgtctcaaaaaaaattttttttttaataaaacaaaatttgtttgaaatcttttaaaaattcaaatgatttttacaagttttaaataagctctccccaaacttgctttatgccttcttattgcttttatgatatatatatgcttggctaactatatttgctttttgctaacaatgctctggggtctttttatgcatttgcatttgctctttcatctctgcttggattattttaaatcattaggaattaagttatctttaaaatttaagtatcttttttcaaaaacattttttaatagaataaaatataatttgatcttattaaa

SEQ ID NO: 31 - DNA, 1123 bp, Homo sapiens
gtgctctgagtttttggtttctgtttcaccttgtgtctgagctggtctgaaggctggttgttcagactgagcttcctgcctgcctgtaccccgccaacagcttcagaagaaggtgactggtggctgcctgaggaataccagtgggcaagagaattagcatttctggagcatctgctgtctgagcagcccctgggtgcgtccactttctgggcacgtgaggttgggccttggccgcctgagcccttgagttggtcacttgaaccttgggaatattgagattatattctcctgccttttaaaaagatggacttcagcagaaatctttatgatattggggaacaactggacagtgaagatctggcctccctcaagttcctgagcctggactacattccgcaaaggaagcaagaacccatcaaggatgccttgatgttattccagagactccaggaaaagagaatgttggaggaaagcaatctgtccttcctgaaggagctgctcttccgaattaatagactggatttgctgattacctacctaaacactagaaaggaggagatggaaagggaacttcagacaccaggcagggctcaaatttctgcctacagggtcatgctctatcagatttcagaagaagtgagcagatcagaattgaggtcttttaagtttcttttgcaagaggaaatctccaaatgcaaactggatgatgacatgaacctgctggatattttcatagagatggagaagagggtcatcctgggagaaggaaagttggacatcctgaaaagagtctgtgcccaaatcaacaagagcctgctgaagataatcaacgactatgaagaattcagcaaagagagaagcagcagccttgaaggaagtcctgatgaattttcaaatgactttggacaaagtttaccaaatgaaaagcaaacctcggggatactgtctgatcatcaacaatcacaattttgcaaaagcacgggagaaagtgcccaaacttcacagcattagggacaggaatggaacacacttggatgcagggtttgagaatgtttttagctggtggcaataaatattagaagcctgcagaatccagctacgaatatagagggttttgctcttg

SEQ ID NO: 32 - DNA, 1157 bp, Homo sapiens
gctcacagtcatcaattatagaccccacaacatgcgccctgaagacagaatgttccatatcagagctgtgatcttgagagccctctccttggctttcctgctgagtctccgaggagctggggccatcaaggcggaccatgtgtcaacttatgccgcgtttgtacagacgcatagaccaacaggggagtttatgtttgaatttgatgaagatgagatgttctatgtggatctggacaagaaggagaccgtctggcatctggaggagtttggccaagccttttcctttgaggctcagggcgggctggctaacattgctatattgaacaacaacttgaataccttgatccagcgttccaaccacactcaggccaccaacgatccccctgaggtgaccgtgtttcccaaggagcctgtggagctgggccagcccaacaccctcatctgccacattgacaagttcttcccaccagtgctcaacgtcacgtggctgtgcaacggggagctggtcactgagggtgtcgctgagagcctcttcctgcccagaacagattacagcttccacaagttccattacctgacctttgtgccctcagcagaggacttctatgactgcagggtggagcactggggcttggaccagccgctcctcaagcactgggaggcccaagagccaatccagatgcctgagacaacggagactgtgctctgtgccctgggcctggtgctgggcctagtcggcatcatcgtgggcaccgtcctcatcataaagtctctgcgttctggccatgacccccgggcccaggggaccctgtgaaatactgtaaaggtgacaaaatatctgaacagaagaggacttaggagagatctgaactccagctgccctacaaactccatctcagcttttcttctcacttcatgtgaaaactactccagtggctgactgaattgctgacccttcaagctctgtccttatccattacctcaaagcagtcattccttagtaaagtttccaacaaatagaaattaatgacactttggtagcactaatatggagattatcctttcattgagccttttatcctctgttctcctttgaagaacccctcactgtcaccttcccgagaataccctaagaccaataaatacttcagtatttc

SEQ ID NO: 33 - DNA, 907 bp, Homo sapiens
gtcggggcgcgcccggaaaccccaaggcccagacggctctcagatccgggggctgcggataaatccccgtaggccgcgggcagcgagatgttgcgttccggtgtgggtgtgggtgtgcctccgacggcgtctcggtgccagtgtcgaggttctttctgcttagctacccggagccgactacggaggaggacacctgagtttacgtctcttccatctgctgctcacctcagctgcctgggtccccgacgagagccaggtgacacttaactccgccatctgcgttttgagcactgttctcataatggagtttcctgatttggggaagcattgttcagaaaagacttgcaagcagctagattttcttccagtaaaatgtgatgcatgtaaacaagatttctgtaaagatcattttccatacgctgcacataagtgtccgtttgcattccagaaggatgttcacgtcccagtatgcccactctgtaatacccccatcccagtaaaaaagggccagataccagacgtggtggttggtgatcacattgacagagactgtgactctcaccctgggaagaagaaagagaagatttttacataccgttgctcaaaagagggctgcaagaagaaagagatgctgcagatggtatgtgcccaatgtcacggcaacttctgtatccagcacagacaccctttggaccacagctgcagacacgggagtcgccccaccatcaaagctgggtgagaagagactccgctgcgatggctcggagcacgcagtagcatgcgtggaagcagcttacactctagtgggaagtggagccccattgagcaccatcccacactggctgctgatcttgtttgttgagggagatgggactataataaaattgaatgctgaagttgaaaaaaa

SEQ ID NO: 34 - DNA, 7751 bp, Homo sapiens
ggggccggggggcggagccttgcgggctggagcgaaagaatgcgggggctgagcgcagaagcggctcgaggctggaagaggatcttgggcgccgccagtctctctctgttgcccaagctggagtgcagtggcacagtcttggctcactgcaacctccacctcctgggtgcaagcgattctcgtgtctcagcctctcaagtagctgggattacaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 35 - DNA, 7796 bp, Homo sapiens
ggggccggggggcggagccttgcgggctggagcgaaagaatgcgggggctgagcgcagaagcggctcgaggctggaagaggatcttgggcgccgccagtctctctctgttgcccaagctggagtgcagtggcacagtcttggctcactgcaacctccacctcctgggtgcaagcgattctcgtgtctcagcctctcaagtagctgggattacagtctttagcaccagttggtgtaggagttgagacctacttcacagtagttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 36 - DNA, 7680 bp, Homo sapiens
ggggccggggggcggagccttgcgggctggagcgaaagaatgcgggggctgagcgcagaagcggctcgaggctggaagaggatcttgggcgccgccagtctttagcaccagttggtgtaggagttgagacctacttcacagtagttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcaacaccaggagagcttctcccaaactccccgttcctgcgtagtccgctttctcttgctgccacattctaaaggcttgatattttccaaatggatcctgttgggaaagaataaaattagcttgagcaacctggctaagatagaggggctctgggagactttgaagaccagtcctgtttgcagggaagccccacttgaaggaagaagtctaagagtgaagtaggtgtgacttgaactagattgcatgcttcctcctttgctcttgggaagaccagctttgcagtgacagcttgagtgggttctctgcagccctcagattatttttcctctggctccttggatgtagtcagttagcatcattagtacatctttggagggtggggcaggagtatatgagcatcctctctcacatggaacgctttcataaacttcagggatcccgtgttgccatggaggcatgccaaatgttccatatgtgggtgtcagtcagggacaacaagatccttaatgcagagctagaggacttctggcagggaagtggggaagtgttccagatagcagggcatgaaaacttagagaggtacaagtggctgaaaatcgagtttttcctctgtctttaaattttatatgggctttgttatcttccactggaaaagtgtaatagcatacatcaatggtgtgttaaagctatttccttgccttttttttattggaatggtaggatatcttggctttgccacacacagttacagagtgaacactctactacatgtgactggcagtattaagtgtgcttattttaaatgttactggtagaaaggcagttcaggtatgtgtgtatatagtatgaatgcagtggggacaccctttgtggttacagtttgagacttccaaaggtcatccttaataacaacagatctgcaggggtatgttttaccatctgcatccagcctcctgctaactcctagctgactcagcatagattgtataaaatacctttgtaacggctcttagcacactcacagatgtttgaggctttcagaagctcttctaaaaaatgatacacacctttcacaagggcaaactttttccttttccctgtgtattctagtgaatgaatctcaagattcagtagacctaatgacatttgtattttatgatcttggctgtatttaatggcataggctgacttttgcagatggaggaatttcttgattaatgttgaaaaaaaacccttgattatactctgttggacaaaccgagtgcaatgaatgatgcttttctgaaaatgaaatataacaagtgggtgaatgtggttatggccgaaaaggatatgcagtatgcttaatggtagcaactgaaagaagacatcctgagcagtgccagctttcttctgttgatgccgttccctgaacataggaaaatagaaacttgcttatcaaaacttagcattaccttggtgctctgtgttctctgttagctcagtgtctttccttacatcaataggtttttttttttttttttggcctgaggaagtactgaccatgcccacagccaccggctgagcaaagaagctcatttcatgtgagttctaaggaatgagaaacaattttgatgaatttaagcagaaaatgaatttctgggaacttttttgggggcgggggggtggggaattcagccacactccagaaagccaggagtcgacagttttggaagcctctctcaggattgagattctaggatgagattggcttactgctatcttgtgtcatgtacccactttttggccagactacactgggaagaaggtagtcctctaaagcaaaatctgagtgccactaaatggggagatggggctgttaagctgtccaaatcaacaagggtcatataaatggccttaaactttggggttgctttctgcaaaaagttgctgtgactcatgccatagacaaggttgagtgcctggacccaaaggcaatactgtaatgtaaagacatttatagtactaggcaaacagcaccccaggtactccaggccctcctggctggagagggctgtggcaatagaaaattagtgccaactgcagtgagtcagcctaggttaaatagagagtgtaagagtgctggacaggaacctccaccctcatgtcacatttcttcaatgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatcctccaactgaccctgatcaacgaagagggattcatgtgtctgtctcagttggttccaaatgaaaccagggagcaggggagttaggaatcgaacaccagtcatgcctactggctctctgctcgagagccaataccctgtgccctccactcatctggatttacaggaactgtcatagtgttcagtattgggtggtgataagcccattggattgtccccttggggggatgagctaggggtgcaaggaacacctgatgagtagataagtggagctcatggtatttcctgaaagatgctaatctatttgccaaacttggtcttgaatgtactgggggcttcaaggtatgggtatatttttcttgtgtccttgcagttagcccccatgtcttatgtgtgtcctgaaaaaataagagcctgcccaagactttgggcctcttgacagaattaaccacttttatacatctgagttctcttggtaagttctttagcagtgttcaaagtctactagctcgcattagtttctgttgctgccaacagatctgaactaatgctaacagatccccctgagggattcttgatgggctgagcagctggctggagctagtactgactgacattcattgtgatgagggcagctttctggtacaggattctaagctctatgttttatatacattttcatctgtacttgcacctcactttacacaagaggaaactatgcaaagttagctggatcgctcaaggtcacttaggtaagttggcaagtccatgcttcccactcagctcctcaggtcagcaagtctacttctctgcctattttgtatactctctttaatatgtgcctagctttggaaagtctagaatgggtccctggtgcctttttactttgaagaaatcagtttctgcctctttttggaaaagaaaacaaagtgcaattgttttttactggaaagttacccaatagcatgaggtgaacaggacgtagttaggccttcctgtaaacagaaaatcatatcaaaacactatcttcccatctgtttctcaatgcctgctacttcttgtagatatttcatttcaggagagcagcagttaaacccgtggattttgtagttaggaacctgggttcaaaccctcttccactaattggctatgtctctggacaagtttttttttttttttttttttaaaccctttctgaactttcactttctatgtctacctcaaagaattgttgtgaggcttgagataatgcatttgtaaagggtctgccagataggaagatgctagttatggatttacaaggttgttaaggctgtaagagtctaaaacctacagtgaatcacaatgcatttacccccactgacttggacataagtgaaaactagccagaagtctctttttcaaattacttacaggttattcaatataaaatttttgtaatggataatcttatttatctaaactaaagcttcctgtttatacacactcctgttattctgggataagataaatgaccacagtaccttaatttctaggtgggtgcctgtgatggttcattgtaggtaaggacattttctctttttcagcagctgtgtaggtccagagcctctgggagaggaggggggtagcatgcacccagcaggggactgaactgggaaactcaaggttctttttactgtggggtagtgagctgcctttctgtgatcggtttccctagggatgttgctgttcccctccttgctattcgcagctacatacaacgtggccaaccccagtaggctgatcctatatatgatcagtgctggtgctgactctcaatagccccacccaagctggctataggtttacagatacattaattaggcaacctaaaatattgatgctggtgttggtgtgacataatgctatggccagaactgaaacttagagttataattcatgtattagggttctccagagggacagaattagtaggatatatgtatatatgaaagggaggttattagggagaactggctcccacagttagaaggcgaagtcgcacaataggccgtctgcaagctgggttagagagaagccagtagtggctcagcctgagttcaaaaacctcaaaactggggaagctgacagtgcagccagccttcagtctgtggccaaaggcccaagagcccctggcaaccaacccactggtgcaagtcctagattccaaaggctgaagaacctggagtctgatgtccaagagcaggaagagtggaagaaagccagaagactcagcaaacaaggtagacagtgtctaccaccatagtggccataccaaagaggctaccgattccttcctgctacctggatccctgaagttgccctggtctctgcaccttctaaacctagttcttaagagctttccattacatgagctgtctcaaagccctccaataaattctcagtgtaagcttctgttgcttgtggacagaaaattctgacagacctaccctataagtgttactgtcaggataacatgagaacgcacaacagtaagtggtcactaagtgttagctacggttattttgcccaaggtagcatggctagttgatgccggttgatggggcttaaacccagctccctcatcttccaggcctctgtactccctattccactaaactacctctcaggtttatttttttaaattcttactctgcaagtacataggaccacatttacctgggaaaacaagaataaaggctgctctgcattttttagaaacttttttgaaagggagatgggaatgcctgcacccccaagtccagaccaacacaatggttaattgagatgaataataaaggaaagactgttctgggcttcccagaatagcttggtccttaaattgtggcacaaacaacctcctgtcagagccagcctcctgccaggaagaggggtaggagactagaggccgtgtgtgcagccttgccctgaaggctagggtgacaatttggaggctgtccaaacaccctggcctctagagctggcctgtctatttgaaatgccggctctgatgctaatcggcgaccctcaggcaagttacttaaccttacatgcctcagttttctcatctggaaaatgagaaccctaggtttagggttgttagaaaagttaaatgagttaagacaagtgcctgggacacagtagcctcttgtgtgtgtttatcattatgtcctcagcaggtcgtagaagcagcttctcaggtgtgaggctggcgcgattatctggagtgggttgggttttctaggatggaccccctgctgcattttcctcattcatccaccagggcttaatggggaatcaaggaatccatgtgtaactgtataataactgtagccacactccaatgaccacctactagttgtccctggcactgcttatacatatgtccatcaaatcaatcctatgaagtagatactgtcttcattttatagatcagagacaattggggttcagagagctgatgtgattttcccagggtcacagagagtcccagattcaggcacaactcttgtattccaagacacaaccactacatgtccaaaggctgcccagagccaccgggcacggcaaattgtgacatatccctaaagaggctgagcacctggtcaggatctgatggctgacagtgtgtccagatgcagagctggagtgggggaggggaaggggggctccttgggacagagaaggctttctgtgctttctctgaagggagcagtctgaggaccaagggaacccggcaaacagcacctcaggtactccaggccctcctggctggagagggctgtggcaatggaaaattagtgccaactgcaatgagtcagcctcggttaaatagagagtgaagaatgctggacaggaacctccaccctcatgtcacatttcttcagtgtgacccttctggcccctctcctcctgacagcggaacaatgactgccccgataggtgaggctggaggaagaatcagtcctgtccttggcaagctcttcactatgacagtaaaggctctctgcctgctgccaaggcctgtgactttctaacctggcctcacgctgggtaagcttaaggtagaggtgcaggattagcaagcccacctggctaccaggccgacagctacatctttcaactgaccctgatcaacgaagagggacttgtgtctctcagttggttccaaatgaaaccagggagcaggggcgttaggaagctccaacaggatggtacttaatggggcatttgagtggagaggtaggtgacatagtgctttggagcccagggagggaaaggttctgctgaagttgaattcaagactgttctttcatcacaaacttgagtttcctggacatttgtttgcagaaacaaccgtagggttttgccttaacctcgtgggtttattattacctcatagggactttgcctcctgacagcagtttatgggtgttcattgtggcacttgagttttcttgcatacttgttagagaaaccaagtttgtcatcaacttcttatttaaccccctggctataacttcatggattatgttataattaagccatccagagtaaaatctgtttagattatcttggagtaagggggaaaaaatctgtaattttttctcctcaactagatatatacataaaaaatgattgtattgcttcatttaaaaaatataacgcaaaatctcttttccttctaaaaaaaaaaaaaaaaaa

SEQ ID NO: 37 - DNA, 2979 bp, Homo sapiens
gggcagcctgctgtcggcttagaggggatgggcagtgtggagggcctggcagagcaagaggactcatccttccaaagggactttctctgggaagcctgctcctcgggccactgcgaaccctctctactctccgaagggaattgtccttcctggcttccactacttccacccctgaatgcacaggcagcccggcccaagtctcccactagggatgcagatggattcggtgtgaagggctggctgctgttgcctccggctcttgaaagtcaagttcagaggcgtgcaaagactccagaattggaggcatgatgaagactctgctgctgtttgtggggctgctgctgacctgggagagtgggcaggtcctgggggaccagacggtctcagacaatgagctccaggaaatgtccaatcagggaagtaagtacgtcaataaggaaattcaaaatgctgtcaacggggtgaaacagataaagactctcatagaaaaaacaaacgaagagcgcaagacactgctcagcaacctagaagaagccaagaagaagaaagaggatgccctaaatgagaccagggaatcagagacaaagctgaaggagctcccaggagtgtgcaatgagaccatgatggccctctgggaagagtgtaagccctgcctgaaacagacctgcatgaagttctacgcacgcgtctgcagaagtggctcaggcctggttggccgccagcttgaggagttcctgaaccagagctcgcccttctacttctggatgaatggtgaccgcatcgactccctgctggagaacgaccggcagcagacgcacatgctggatgtcatgcaggaccacttcagccgcgcgtccagcatcatagacgagctcttccaggacaggttcttcacccgggagccccaggatacctaccactacctgcccttcagcctgccccaccggaggcctcacttcttctttcccaagtcccgcatcgtccgcagcttgatgcccttctctccgtacgagcccctgaacttccacgccatgttccagcccttccttgagatgatacacgaggctcagcaggccatggacatccacttccatagcccggccttccagcacccgccaacagaattcatacgagaaggcgacgatgaccggactgtgtgccgggagatccgccacaactccacgggctgcctgcggatgaaggaccagtgtgacaagtgccgggagatcttgtctgtggactgttccaccaacaacccctcccaggctaagctgcggcgggagctcgacgaatccctccaggtcgctgagaggttgaccaggaaatacaacgagctgctaaagtcctaccagtggaagatgctcaacacctcctccttgctggagcagctgaacgagcagtttaactgggtgtcccggctggcaaacctcacgcaaggcgaagaccagtactatctgcgggtcaccacggtggcttcccacacttctgactcggacgttccttccggtgtcactgaggtggtcgtgaagctctttgactctgatcccatcactgtgacggtccctgtagaagtctccaggaagaaccctaaatttatggagaccgtggcggagaaagcgctgcaggaataccgcaaaaagcaccgggaggagtgagatgtggatgttgcttttgcacctacgggggcatctgagtccagctccccccaagatgagctgcagccccccagagagagctctgcacgtcaccaagtaaccaggccccagcctccaggcccccaactccgcccagcctctccccgctctggatcctgcactctaacactcgactctgctgctcatgggaagaacagaattgctcctgcatgcaactaattcaataaaactgtcttgtgagctgatcgcttggagggtcctctttttatgttgagttgctgcttcccggcatgccttcattttgctatggggggcaggcaggggggatggaaaataagtagaaacaaaaaagcagtggctaagatggtatagggactgtcataccagtgaagaataaaagggtgaagaataaaagggatatgatgacaaggttgatccacttcaagaattgcttgctttcaggaagagagatgtgtttcaacaagccaactaaaatatattgctgcaaatggaagcttttctgttctattataaaactgtcgatgtattctgaccaaggtgcgacaatctcctaaaggaatacactgaaagttaaggagaagaatcagtaagtgtaaggtgtacttggtattataatgcataattgatgttttcgttatgaaaacatttggtgcccagaagtccaaattatcagttttatttgtaagagctattgcttttgcagcggttttatttgtaaaagctgttgatttcgagttgtaagagctcagcatcccaggggcatcttcttgactgtggcatttcctgtccaccgccggtttatatgatcttcatacctttccctggaccacaggcgtttctcggcttttagtctgaaccatagctgggctgcagtaccctacgctgccagcaggtggccatgactacccgtggtaccaatctcagtcttaaagctcaggcttttcgttcattaacattctctgatagaattctggtcatcagatgtactgcaatggaacaaaactcatctggctgcatcccaggtgtgtagcaaagtccacatgtaaatttatagcttagaatattcttaagtcactgtcccttgtctctctttgaagttataaacaacaaacttaaagcttagcttatgtccaaggtaagtattttagcatggctgtcaaggaaattcagagtaaagtcagtgtgattcacttaatgatatacattaattagaattatggggtcagaggtatttgcttaagtgatcataattgtaaagtatatgtcacattgtcacattaatgtcaaaaaaaaaaaaaaaaaa

SEQ ID NO: 38 - DNA, 20 bp, Homo sapiens
cccttacacatcctactcct

SEQ ID NO: 39 - DNA, 21 bp, Homo sapiens
cagcaataacagacgtaaccg

SEQ ID NO: 40 - DNA, 133 bp, Homo sapiens
cccttacacatcctactccttatttaaaagttctatttgtgacttttcatttcctgaaagtttaaaaatacaatttgagaatgtttataatacattctctcctgtcttttcacggttacgtctgttattgctg

SEQ ID NO: 41 - DNA, 20 bp, Homo sapiens
cacacctttcacaagggcaa

SEQ ID NO: 42 - DNA, 20 bp, Homo sapiens
ctccatctgcaaaagtcagc

SEQ ID NO: 43 - DNA, 141 bp, Homo sapiens
cacacctttcacaagggcaaactttttccttttccctgtgtattctagtgaatgaatctcaagattcagtagacctaatgacatttgtattttatgatcttggctgtatttaatggcataggctgacttttgcagatggag

SEQ ID NO: 44 - DNA, 20 bp, Homo sapiens
acaacgagctgctaaagtcc

SEQ ID NO: 45 - DNA, 20 bp, Homo sapiens
gatagtactggtcttcgcct

SEQ ID NO: 46 - DNA, 129 bp, Homo sapiens
acaacgagctgctaaagtcctaccagtggaagatgctcaacacctcctccttgctggagcagctgaacgagcagtttaactgggtgtcccggctggcaaacctcacgcaaggcgaagaccagtactatc

SEQ ID NO: 47 - DNA, 20 bp, Homo sapiens
gtggaaggtgattgtttcgc

SEQ ID NO: 48 - DNA, 20 bp, Homo sapiens
ggatcttcccagcttcaatg

SEQ ID NO: 49 - DNA, 149 bp, Homo sapiens
gtggaaggtgattgtttcgctggtcctgttgatgcctggcccctgtgatgggctgtttcgctccctatacagaagtgtttccatgccacctaagggagactcaggacagccattatttctcaccccttacattgaagctgggaagatcc

SEQ ID NO: 50 - DNA, 20 bp, Homo sapiens
ccgtaatgggattttctggg

SEQ ID NO: 51 - DNA, 19 bp, Homo sapiens
gcttaaagtgcttgggtct

SEQ ID NO: 52 - DNA, 113 bp, Homo sapiens
ccgtaatgggattttctggggtacctggcctggtgtaagtgaggcacaccctggtggctacaagtcctccttcaaagaggctaagatgatgatcagacccaagcactttaagc

SEQ ID NO: 53 - DNA, 24 bp, Homo sapiens
cttctcacttcatgtgaaaactac

SEQ ID NO: 54 - DNA, 23 bp, Homo sapiens
ccatattagtgctaccaaagtgt

SEQ ID NO: 55 - DNA, 148 bp, Homo sapiens
cttctcacttcatgtgaaaactactccagtggctgactgaattgctgacccttcaagctctgtccttatccattacctcaaagcagtcattccttagtaaagtttccaacaaatagaaattaatgacactttggtagcactaatatgg

SEQ ID NO: 56 - DNA, 20 bp, Homo sapiens
ctgtaaggcagatgatcgtc

SEQ ID NO: 57 - DNA, 21 bp, Homo sapiens
tgttcagtgctttctgacaag

SEQ ID NO: 58 - DNA, 113 bp, Homo sapiens
ctgtaaggcagatgatcgtccccgtatgatgattgtcagaagacaggactaagtagcagagaatagctaagagataaattgggctggggaaacttgtcagaaagcactgaaca

SEQ ID NO: 59 - DNA, 20 bp, Homo sapiens
ctcagggctgtgtgtactaa

SEQ ID NO: 60 - DNA, 20 bp, Homo sapiens
gtcaaagtctctacccacag

SEQ ID NO: 61 - DNA, 98 bp, Homo sapiens
ctcagggctgtgtgtactaatacagactatgtcacccaatgcatatccaacatgtgctcagggaataatccagaaaaactgtgggtagagactttgac

SEQ ID NO: 62 - DNA, 20 bp, Homo sapiens
ggtttagttggactggatgg

SEQ ID NO: 63 - DNA, 20 bp, Homo sapiens
aatagccgtaagcctgtctc

SEQ ID NO: 64 - DNA, 114 bp, Homo sapiens
ggtttagttggactggatgggatctggaaataaatatttcccaaaagttgtccatgggatcctagcagtcagggttgagaattgctaagttatagagacaggcttacggctatt

SEQ ID NO: 65 - DNA, 19 bp, Homo sapiens
ggagtctaattgcagttcc

SEQ ID NO: 66 - DNA, 20 bp, Homo sapiens
ctgttactagtgacgtttgg

SEQ ID NO: 67 - DNA, 88 bp, Homo sapiens
ggagtctaattgcagttccctgagccatgtgcctttctcttcactgaggactgccccattcttgagtgccaaacgtcactagtaacag

SEQ ID NO: 68 - DNA, 20 bp, Homo sapiens
gagtggtctgcagctatact

SEQ ID NO: 69 - DNA, 20 bp, Homo sapiens
aagcatgagtattgggaagg

SEQ ID NO: 70 - DNA, 143 bp, Homo sapiens
gagtggtctgcagctatacttccagtatctccaagatatgactgtactttttcttgtatttatttactggtgacagcttttctttttatagttatatcacttagaataataattttgttatttccttcccaatactcatgctt

SEQ ID NO: 71 - DNA, 18 bp, Homo sapiens
gtgtaccctgactcgttc

SEQ ID NO: 72 - DNA, 21 bp, Homo sapiens
agagtgacagtgcatacaatg

SEQ ID NO: 73 - DNA, 119 bp, Homo sapiens
gtgtaccctgactcgttctctggggtttccctcttctctctttccacggaaggttgtgatgaagaggaagtcttaaagtccctaaagttctcccttttcattgtatgcactgtcactct

SEQ ID NO: 74 - DNA, 21 bp, Homo sapiens
acttctgtatccagcacagac

SEQ ID NO: 75 - DNA, 21 bp, Homo sapiens
ccctcaacaaacaagatcagc

SEQ ID NO: 76 - DNA, 203 bp, Homo sapiens
acttctgtatccagcacagacaccctttggaccacagctgcagacacgggagtcgccccaccatcaaagctgggtgagaagagactccgctgcgatggctcggagcacgcagtagcatgcgtggaagcagcttacactctagtgggaagtggagccccattgagcaccatcccacactggctgctgatcttgtttgttgaggg

SEQ ID NO: 77 - DNA, 20 bp, Homo sapiens
gctaaattccacactaccac

SEQ ID NO: 78 - DNA, 20 bp, Homo sapiens
ttgagtgcagcggtgtgaac

SEQ ID NO: 79 - DNA, 157 bp, Homo sapiens
gctaaattccacactaccacattaaaaaaattagaaagtagccacgtatggtggctcatgtctataatcccagcactttgggaggttgaggtgggaggattgcttgaacccaagaggtcaaggctgcagtgagccatgttcacaccgctgcactcaa

SEQ ID NO: 80 - DNA, 19 bp, Homo sapiens
tgacagagccagtgggaag

SEQ ID NO: 81 - DNA, 19 bp, Homo sapiens
caattctcctacctcaacc

SEQ ID NO: 82 - DNA, 224 bp, Homo sapiens
tgacagagccagtgggaagatacagaaaaagaggggctgggctgggcgcggtggttcacgcctgtaatcccagcactttgggaggccaaggagggtggatcacctgaggtcaggagttagaggccagcctggcgaaaccccatctctactaaaaatacaaaatccaggcgtggtggcacacacctgtagtcccagctactcaggaggttgaggtaggagaattg

SEQ ID NO: 83 - DNA, 19 bp, Homo sapiens
aggtgtgagctactgtacc

SEQ ID NO: 84 - DNA, 18 bp, Homo sapiens
ggattacaggcatgcaac

SEQ ID NO: 85 - DNA, 115 bp, Homo sapiens
aggtgtgagctactgtacccagccttaacctgtttcacagttgattatacttcatgctgttttccagcatggtattattaagggatttaaagtttgggttgcatgcctgtaatcc

(19) United States
(12) Patent Application Publication
(10) Pub. No.: US 2011/0076685 A1
(43) Pub. Date: Mar. 31, 2011

(54) METHOD FOR IN VITRO DETECTION AND DIFFERENTIATION OF PATHOPHYSIOLOGICAL CONDITIONS

(75) Inventors: Eva Moeller, Jena (DE); Britta Wlotzka, Erfurt (DE); Andriy Ruryk, Jena (DE)

(73) Assignee: SIRS-LAB GMBH, Jena (DE)

(21) Appl. No.: 12/888,948

(22) Filed: Sep. 23, 2010

Related U.S. Application Data
(60) Provisional application No. 61/245,181, filed on Sep. 23, 2009.

(51) Int. Cl. C12Q 1/68; C07H 21/00

(52) U.S. Cl. 435/6; 536/24.33; 536/23.1

(57) ABSTRACT

The present invention relates to the use of defined polynucleotides to form at least one multi-gene biomarker for producing a multiplex assay as a tool for in vitro detection and/or early detection and/or differentiation and/or progress monitoring and/or evaluation of pathophysiological conditions of a patient, the pathophysiological condition selected from the group consisting of: SIRS, sepsis and its degrees of severity; sepsis-like conditions, septic shock, bacteremia, infective/non-infectious multiorgan failure, early detection of these conditions; focus control, control of surgical rehabilitation of the infection focus; responders/non-responders for a specific therapy, treatment monitoring; distinction between infectious and non-infectious etiology of systemic reactions of the organism, such as SIRS, sepsis, postoperative complications, chronic and/or acute organ dysfunction, shock response, inflammatory response and/or trauma.

The present invention relates to a method for in vitro detection and/or early recognition and/or differentiation and/or monitoring the course of pathophysiological conditions according to claim 1, the use of at least three polynucleotides to form at least one multi-gene biomarker for producing a multiplex assay as a tool to assess whether a patient is presenting a pathophysiological condition, and/or to determine the severity and/or for early detection and/or to change and/or follow-up of pathophysiological conditions according to claim 5, a use according to claim 12, primers for carrying out the invention according to claim 16, and a kit for carrying out the method according to claim 17.

In particular, the present invention describes the use of polynucleotides for detecting gene activity of at least one multi-gene biomarker to produce a resource for diagnosis in patients with certain pathophysiological conditions such as sepsis and sepsis-like conditions, with characteristics similar to an “In Vitro Diagnostic Multivariate Index Assay” (IVDMIA).

Sepsis (blood poisoning) is a life-threatening infection, which can affect the entire body. It is associated with high mortality, is becoming increasingly prevalent and affects people at any age. Sepsis endangers medical progress in many areas of medicine and consumes a large part of health care resources. The mortality of severe sepsis has not really improved in recent decades. The last two steps in innovation after introducing the blood culture (around. 1880) were the introduction of antibiotics over 60 years ago and the beginning of the intensive care about 50 years ago. To achieve a similarly significant advance in treatment today, new types of diagnosis must be made available.

Sepsis is caused by infectious agents. As there is no specific therapy for sepsis, the success of treatment depends largely on the successful treatment of the underlying infection and the quality of intensive care. Crucial for survival is the early administration of an antibiotic, which also fights successfully against the causative pathogen [Kumar et., 2006]. Deficits of sepsis diagnosis, however, delay the onset of treatment and limit the choice of an appropriate antibiotic. Since the identification of the sepsis pathogen, using the current methods of blood culture, succeeds in less than 25% of sepsis cases, and since findings in the case of pathogen detection only are available after 2-3 days, the initial choice of the antibiotic or fungicide (substances directed against fungi) has to be “calculated”, which means chosen based on suspicion. In 20-30% of cases, this choice is not correct.

Other causes for the delay in treatment lie in the misinterpretation of disease symptoms and laboratory values. Improved diagnostic tools that simplify and accelerate the diagnosis of sepsis may lead to a significant reduction in sepsis mortality rates and a shortening of the treatment time. Medical societies acknowledge the shortcomings of previous sepsis diagnosis in surveys of North American and European intensive care providers [Marshall et. al., 2003]. al., 2003]. The concerned citizens initiative “German Sepsis Aid Association” and the German Sepsis Society complain about the shortcomings.

With the development of market-ready in-vitro diagnostics in the field of molecular diagnostics, the Food and Drug Administration (FDA) of the United States of America published a draft directive on Jul. 26, 2007. This directive provides recommendations, definitions and guidance for the development and approval process. Furthermore, specifications for the new class of “in vitro diagnostic multivariate index assays” (IVDMIA) were proposed. Features of these assays are:
1) The combination of several individual values using an interpretation step, to get an individual patient-specific output value in the form of an index, score or a classification. This value can be used for diagnostic statements, mitigation, treatment or prevention of a disease.
2) The result obtained is derived from the measurements in a manner that does not allow to refer back to the actual measurement data. Therefore, the result can not be confirmed or reconstructed by end-users.
3) Thus it is necessary to make all information for interpreting the test results available to the user.

An infection is associated with the characteristic intake of pathogens, their proliferation in the organism, and the associated induction of pathophysiological and symptomatic reactions. In contrast, disease symptoms are not exhibited in a colonization of the host organism.

In the course of an infection, a confrontation between the pathogens and the body's own defense occurs within the body. The non-specific defense concerns the body's own germicidal substances, which are dissolved in the blood (humoral), as well as granulocytes and macrophages, which have limited capability to eliminate foreign objects, invaders and cellular debris. The principle of the specific defense is to mark pathogens and invaders with antibodies circulating in the blood, so that they can then subsequently be destroyed with T lymphocytes.

Following the spread of a pathogen, several disease processes can be initiated. On the one hand, there are defensive reactions such as fever, vasodilation, and/or encapsulations. This can lead to a damaging or destruction of tissues, organs or organ systems such as multiorgan failure (MOV). Depending on the pathogen, the causative organism can excrete toxins or exotoxins, leading sometimes to acute reactions of the host response. Another possibility is that pathogen components, called endotoxins, have the effect of a poison, in case of a germicidal effect.

In the case of a limitation of infection events to one region of the organism, one refers to this as a local infection, such as in the case of abscesses or wound infections. The symptoms of local infection are redness, swelling, pain and limited function. If however the pathogens spread through the bloodstream or lymph system to the whole body, this is referred to as general or systemic infection. From the beginning of infection up to onset of reactions (symptoms) different time periods are observed, depending on the individual, which period is known as the incubation period.

The diversity in nature, symptoms, severity and patterns of infections make a specific detection or a differential diagnosis regarding sterile inflammatory afflictions very difficult in clinical routine and often imprecise. Herein is seen a major reason for frequent serious infectious complications in many different indications and medical disciplines. There is a great medical need in a variety of medical disciplines to detect such infectious complications with adequate sensitivity and specificity, to treat them with appropriate clinical interventions, and to make available a monitor or follow-up the individual clinical measures for the treatment of infectious complications. This is particularly true for the transition from local to generalized infection, which in a short time leads to (life-threatening conditions.

The distinction between systemic inflammatory and infectious disease-related conditions is playing an important role for the clinical decisions to treat patients and subsequent observation not only in sepsis but also in a number of other indications. In this context, the treatment of acute and chronically ill patients and the peri-operative monitoring can be included. It is known that in the case of acute pancreatitis, an infection significantly worsens the prognosis of a lethal outcome by 16% to 40%. In the event of a complex super-infection there is an elevated risk of sepsis with a mortality of up to 90%. Furthermore, the observation of a course of intra-abdominal inflammation and/or infection in chronically ill, post surgical and trauma patients is important. There are difficulties even today of a clear clinical diagnosis of intra-abdominal infections. The course of monitoring chronic illnesses, such as in patients with liver cirrhosis or renal failure, is of clinical relevance because these patients may be predestined, depending on organ decompensation, to take an inflammatory and/or infectious course of disease. In particular, renal failure patients with peritoneal dialysis are prone to chronic inflammations and infections [Blake, 2008]. Of particular interest is the observation of patients with liver cirrhosis, as these may spontaneously develop bacterial peritonitis, which has a high mortality. [Koulaouzidis et al. 2009]. 2009]. The diagnosis of secondary peritonitis in a post-treatment is of great clinical value and can greatly influence the success of surgery. Postoperative infections are still a major problem today in surgical treatment. One percent of laparotomies carried out result in complications after surgery. Here, the complication rates vary considerably between the surgical procedures. In particular, insufficient suturing can result, in operations on the gastro-intestinal tract, in fulminant spread of bacteria into the sterile abdominal cavity. Infectious occurrences play a role, among others, in the post-operative follow-up treatment after transplantation, thoracotomies, limb and joint corrections and neurosurgical operations.

The person of ordinary skill in the art is aware that these examples are merely illustrative, and that there are numerous other fields of application, for which the identification of the infectious complication is of great importance. The present invention provides a solution to this diagnostic problem.

The present invention relates in particular to genes and/or their fragments and their use in the production of multi-gene biomarkers, which are specific for a condition and/or examination or research object.

The invention also relates to PCR primers and probes derived from the marker gene for hybridization or replication methods.

As before, sepsis is one of the most difficult diseases in modern intensive care practice, which provides a challenge for the clinical practitioners not only with respect to therapy but also in the diagnosis. Despite advances in pathophysiologic understanding and supportive treatment of critical care patients, generalized inflammatory conditions such as SIRS and sepsis are diseases that occur very frequent in patients in intensive care and significantly contribute to mortality [Marshal et al., 2003; Alberti et al., 2003]. The mortality rate is about 20% in SIRS, about 40% in sepsis and increases up to 70-80% upon the occurrence of multiorgan dysfunction in [Brun-Buisson et al., 1995; Le Gall et al., 1995; Brun-Buisson et al., 2003]. The morbidity and contribution to lethality of SIRS and sepsis is of interdisciplinary clinical and medical importance, as this will, increasingly, put at risk the gains in treatment results achieved in advanced therapy in numerous medical fields (eg, trauma, neurosurgery, heart and lung surgery, abdominal surgery, transplant, hematology/oncology, etc.), since without exception an increase in disease risk for SIRS and sepsis is imminent. This is also reflected in the continuous increase in the incidence of sepsis: from 1979-1987, an increase of 139% in cases of illness, from 73.6 to 176 per 100,000 registered hospital patients, was recorded [MMWR Morb Mortal Wkly Rep 1990]. The reduction of morbidity and mortality for a large number of seriously ill patients will thus require simultaneous progress in the prevention, treatment, and in particular the detection and monitoring of sepsis and severe sepsis.

Over time, the term sepsis has substantially received a significant change of definition. Infection or the urgent suspicion of infection are still an essential part of current sepsis definitions. Special consideration is thereby given to the description of organ malfunction remote from the infection site in the framework of the inflammatory host response. In the meantime, in the professional literature, the criteria of the consensus conference of the American College of Chest Physicians/Society of Critical Care Medicine Consensus Conference (ACCP/SCCM) in 1992 has been most widely adopted as the definition of the sepsis concept [Bone et al. 1992]. According to these criteria, distinctions are made between the clinically defined degrees of severity “systemic inflammatory response syndrome” (SIRS), “sepsis”, “severe sepsis” and “septic shock”. With regard to SIRS, this is defined as the systemic inflammatory response of the system to a noninfectious stimulus. This requires satisfying at least two of the following clinical criteria: fever >38° C. or hypothermia <36° C., leukocytosis >12 g/l or leukopenia <4 g/l or a shift to the left in the differential blood count, a heart rate of more than 90/min, tachypnea >20 breaths/min or a PaCO₂ (partial pressure of carbon dioxide in arterial blood)<4.3 kPa. This definition has high sensitivity but low specificity. For intensive care concerns, it is of little help because basically every intensive care patient at some time, at least for a short time, satisfies these SIRS criteria.

With regard to sepsis, this is defined in terms of clinical conditions which meet the SIRS criteria and for which the cause is proven to be an infection, or of which an infection is at least very likely. An infection is defined as a pathological process in which invasion of pathogens, or potentially pathogenic organisms, are found in a normally sterile tissue. If the body fails to limit these infections to the point of origin, then the pathogens or their toxins induce inflammation in the body's organs or tissues remote from the site of infection. An immediate intensive treatment, the targeted administration of antibiotics and the surgical sanitization of the infectious focus, are needed to achieve recovery. Severe sepsis is characterized by the additional occurrence of organ malfunction. Organ malfunctions frequently involve changes in consciousness, an oliguria, lactic acidosis, or a sepsis-induced hypotension, systolic blood pressure of less than 90 mmHg and a pressure drop by more than 40 mmHg from baseline. If such hypotension is not corrected by the administration of crystalloids and/or colloids and if the patient also requires catecholamines, this is referred to as septic shock. This is found in about 20% of all sepsis patients.

Many doctors agree that the consensus criteria according to [Bone et al. 1992], do not correspond with any specific definition of sepsis. A survey done by the European Society of Intensive Care Medicine (ESICM) shows that 71% of surveyed physicians had uncertainty in the diagnosis of sepsis despite many years of clinical experience [Poeze et., 2003]. The attempt to gain acceptance of a uniform terminology has met with mixed acceptance in clinical implementation. In particular, the progress in understanding the pathophysiology of sepsis caused various person of ordinary skill in the arts to search for an appropriate modification of the existing definitions. The definitions of sepsis, severe sepsis and septic shock and were confirmed, and the definitions were determined to be useful for clinicians and researchers. However, the diagnostic criteria of sepsis were significantly expanded to include the clinical aspect of host defense. The International Sepsis 2001 conference also proposed a new concept (called PIRO) to describe sepsis, which were compiled from the criteria: predisposition, infection, immune response (response) and organ dysfunction [Levy et al., 2003]. Despite a new definition of SIRS/sepsis with the acronym PIRO [Opal et al., 2005], most studies still use the ACCP/SCCM consensus conference of 1992 [Bone et al., 1992] to classify their patients.

Several approaches to the diagnosis of SIRS and sepsis have been developed. These approaches can be divided into three groups.

The first group contains scoring systems such as APACHE, SAPS and SIRS, which can stratify the patients on the basis of a wide variety of physiological indices. While in some studies a diagnostic potential could be proven for the APACHE II score, other studies have shown that APACHE II and SAPS II do not differentiate between sepsis and SIRS [Carrigan et al., 2004].

The second group contains protein markers that are detected from serum and plasma. These include, for example, CA125, S100B, copeptin, glycine N-acyltransferase (GNAT), protachykinin and/or its fragments, aldose 1-epimerase (mutarotase), Chp, carbamoylphosphate synthetase 1, LASP-1 (Brahms Diagnostics GmbH, Germany), IL Ra-1, MCP-1, MPIF-1, TNF-alpha, TNF-R1, MIG, BLC, HVEM, IL-10, IL-15, MCP-2, M-CSF, MIP-3b, MMP-9, PARC, ST-2; IL-6, sIL-2R, CD141, MMP-9, EGF, ENA-78, EOT, Gro-beta, IL-1b, leptin, MIF, MIP-1a, OSM, protein C, P-selectin, and HCC4 (Molecular Staging, Inc., USA) or CD 14 antigen, lipopolysaccharide-binding sites on the proteins alkaline phosphatase and Inter-alpha-trypsin inhibitor (Mochida Pharm Co, Ltd., Japan). Despite the large quantity of patented biomarkers, only few have succeed in clinical practice. Among these, Sprocalcitonin (PCT, BRAHMS) and C-reactive protein (CRP, Eli Lilly), appear to be the markers best able to distinguish between infectious and non-infectious causes of SIRS.

Procalcitonin is a 116 amino acid peptide that plays a role in the inflammatory responses. This marker has over time been more and more used as a new infection marker in intensive care units [Sponholz et al., 2006]. This marker is regarded as marker of infection and serves to define the severity of sepsis, wherein the dynamics of the values is more important than the absolute values themselves, in order to distinguish, for example, in heart surgery patients, between infectious and non-infectious complications [Sponholz et al., 2006]. Despite the wide acceptance of the biomarker PCT, international studies have shown that the achieved sensitivities and specificities of the sepsis marker PCT are still insufficient to distinguish between a systemic bacterial SIRS, ie sepsis, and a non-bacterial SIRS [Ruokonen et al., 1999; Suprins et al. 2000; 2000; Ruokonen et al., 2002; Tang et al., 2007a]. Ruokonen et al., 2002; Tang et al., 2007a]. The meta-analysis by Tang and colleagues [Tang et al., 2007a], in which 18 studies were considered, shows that the PCT is poorly suited to discriminate SIRS from sepsis. In addition, the authors stressed that PCT had a very weak diagnostic accuracy with an Odd Ratio (OR) of 7.79. As a rule, the authors mention that an OR <25 is not meaningful, between 25-100 is helpful, and in the case of more than 100 it is highly accurate [Tang et al., 2007a].

C-reactive protein (CRP) is a 224 amino acid protein that plays a role in inflammatory reactions. The CRP measurement serves as an indicator of the progress of the disease as well as to effectiveness of the chosen therapy.

Several reports have described that in the critical care area PCT is more suited as a marker for diagnostics than CRP [Sponholz et al., 2006; Kofoed et al., 2007]. In addition, PCTs are considered better suited than CRP for distinguishing a non-infectious vs. infectious SIRS and to distinguish bacterial infection versus viral [Simon et al., 2004].

It is obvious to the person of ordinary skill in the art that the solution provided according to this invention can be combined with biomarkers such as PCT or CRP, but not limited to these, in order to expand the diagnostic value.

The third group includes biomarkers or profiles, which were identified on the transcriptome level. These molecular parameters should allow a better correlation of the molecular inflammatory/immunological host response with the degree of severity of the sepsis, and also provide indications for individual prognosis. Various scientific groups and commercial organizations are currently intensively searching for biomarkers such as, for example, changes in cytokine concentrations in the blood caused by bacterial cell wall components such as lipopolysaccharides [Mathiak et al., 2003], or use of gene expression profiles in a blood samples to determine differences between surviving and non-surviving sepsis patients [Pachot et al., 2006]. Gene expression profiles or classifiers are suitable to determine the seriousness levels of sepsis [WO 2004/087949], the distinction between a local and systemic infection [unpublished DE 10 2007 036 678.9], identifying the source of infection [WO 2007/124820] or gene expression signatures for distinguishing between various etiologies and pathogen-associated signatures [Ramilo et al. 2007]. However, due to insufficient specificity and sensitivity of the consensus criteria according to [Bone et al., 1992], of the currently available protein markers and also due to the time required for blood culture for proof of source of infection, there is an urgent need for new procedures which take into consideration the complexity of the disease. Many gene expression studies are known which are based on either individual genes and/or combinations of genes than are identified as classifiers, and the art is also replete with numerous descriptions of statistical methods to derive a score and/or index [WO03084388, U.S. Pat. No. 6,960,439].

There is consensus today that complex diseases can only be meaningfully described using several parameters.

Increasingly, molecular signatures are making inroads into clinical diagnostics, especially in complex diseases, which can not be detected with conventional biomarkers, and also for the assessment of risks to patients and identification of responders in the use of drugs and therapies. The following list illustrates the current status and diagnostic applications of gene expression.

1) The microarray-based, 70 gene comprising signature MammaPrint (Agendia, NL) allows the making of a prognosis of the risk of the recurrence and risk of metastasis in women with breast cancer. It is investigated whether the risk of development of remote metastases in the following years can be classified as low or high, and thus whether they could benefit from chemotherapy. The approval of this test by the FDA brought with it the development of guidelines for a new class of diagnostic tests, so-called IVDMIA (in vitro diagnostic multivariate index assay). The MammaPrint signature is measured and calculated on a micro-array in the laboratories of the manufacturer.
2) With formalin-fixed tissue samples the likelihood of recurrence of breast cancer in patients is assessed by means of the Oncotype DX multigene assay (Genomic Health, USA), and the responsiveness of the patients to chemotherapy is tested. 21 genes are combined as a “Recurrence Score”. The measurement takes place in the premises of the company, and the technology TaqMan-PCR is also used.
3) The AlloMap gene expression test of the XDx Company (USA) is used in to monitor possible rejection in heart transplant patients, which occurs in about 30% of patients within one year of the procedure. Until now, several biopsies were necessary for diagnosis. The test is based on 11 quantitative PCR assays (plus 9 additional control samples and references) using the TaqMan technology (Hoffman-La Roche) on the premises of the manufacturer. The sample material is blood. The results are reliable beginning just two months after the transplant, and allow prediction of rejection episodes for the next 80 days in advance.

One common feature of these tests is that the diagnostic addressed inquiry requires several days from examination times before the availability of the result. For diagnostic tests for the treatment of sepsis, however, information must be available within one working day.

In the use of gene markers for identifying a pathophysiological condition, the quantities of the corresponding mRNA which are always present in a sample, the gene expression level, are quantified. By the determined information of gene expression levels, the respective over- or under-expression of these mRNAs, with reference to a control condition, or based on control genes, is determined experimentally. The finding of over- or under-expression can be seen as an analog to the determination of the concentration of a protein biomarker.

Several applications of gene expression profiles are known in the state of the art.

Pachot and colleagues demonstrated the use of expression signatures for the course of evaluating patients with septic shock. Here molecular differences are found, which reflect the restoration of a functioning immune system in the survivors. 28 marker genes with functions in the innate immune system show within the first day after diagnosis of septic shock with high sensitivity (100%) and specificity (88%) whether an immune paralysis is reversible and thus predicts survival of the patient. However, the patient population was too small (38) during this investigation to create a robust profile and a validation of this study by an independent dataset has not yet taken place. The state of the art contains numerous studies to identify gene expression markers [Tang et al., 2007b] or gene expression profiles for the finding of a systemic infection [Johnson et al., 2007].

Tang and colleagues [Tang et al., 2007b] looked in a particular blood cell population, the neutrophils, for a signature which makes it possible to distinguish between patients with SIRS and sepsis. 50 markers from this cell population suffice to reproduce the immune response to systemic infection and enable new discoveries into the pathophysiology and the involved signaling pathways.

The classification of patients as to with and without sepsis succeeds with high reliability (PPV 88% and 91% in training and testing data sets). The applicability for clinical diagnosis is, however, limited by the fact that in blood the signatures of signals from other blood cell types can be overlaid. Regarding the practical applicability, the preparation of these blood cell populations is associated with a significantly increased burden. The strength of this study was also limited for practical applications because the patient selection was very heterogeneous. Patients were included the study which had very different serious concomitant diseases such as e.g., up to 11% to 16% tumors, or were subjected to very different therapeutic measures (e.g., 27% to 64% vasopressor therapy), whereby the gene expression profiles were strongly affected.

### Johnson

Johnson and colleagues [Johnson et al., 2007] describe that in a group of trauma patients the expression of sepsis can be measured based on molecular alterations already to 48 hours before the clinical diagnosis. The trauma patients were studied over several days. Some of the patients developed sepsis. Noninfectious SIRS patients were compared with pre-septic patients. The identified signature of 459 transcripts consisted of markers of the immune response and inflammation markers. The sample was whole blood, the analysis was performed on a microarray. It was unclear whether this signature could be expanded to other populations of septic or pre-septic patients. A classification and diagnostic utility of this signature was not shown.

Furthermore, other signatures are described in the prior art, for example, the response of the host to infection.

The specificity of the host response to different pathogens has been investigated in several experimental systems so far. In no study, however, were gene expression profiles and/or test signatures from whole blood of sepsis patients described.

The goal of Feezor and colleagues [Feezor et al., 2003] was to identify differences between infections with gram-negative and gram-positive pathogens. Blood samples from three different donors were stimulated ex vivo with E. coli LPS and heat-inactivated S. aureus. Using microarray technology, gene expression studies were carried out. The working group found on the one hand genes which were upregulated after the S. aureus stimulation and downregulated after LPS stimulation, and on the other hand genes which were more strongly expressed after treatment with LPS than after the addition of heat-inactivated S. aureus bacteria. At the same time, many genes were up-regulated to the same degree by gram-positive and gram-negative stimulation. This example relates to the cytokines TNF-α, IL-1β and IL-6. The differentially expressed genes were unfortunately not published by name, so that only an indirect comparison is possible with other results. In addition to the gene expression Feezor et al. studied the plasma concentrations of some cytokines. It was found that the gene expression data did not necessarily correlate with the plasma concentrations. In gene expression, the quantity of mRNA is measured. This is, however, subject to or liable to the posttranscriptional regulation of protein synthesis, from which the observed differences may have resulted.

The most interesting publications on this subject was published by a Texas research group of Ramilo [Ramilo et al. 2007]. Here, gene expression studies were also carried out on human blood cells, which uncovered molecular differences in host response to various pathogens. For this, critically ill pediatric patients with acute infections such as acute respiratory infections, urinary tract infections, bloodstream infections, local abscesses, bone and joint infections and meningitis were studied. Microarray experiments were carried out with RNA samples, which were isolated from peripheral blood mononuclear cells from ten patients with E. coli- or S. aureus infection. The identification of the pathogens was carried by blood culture. On the basis of the training data set 30 genes were identified by which the causative pathogens could be diagnosed with high accuracy.

Despite the numerous published studies and the therein described individual signatures that make up the state of the art, none allows a diagnosis of sepsis and/or sepsis-like condition. None of these publications provides the reliability, accuracy and robustness of the invention disclosed here. These studies are focused on identifying the “best” multi-gene biomarker (classifier) from a scientific perspective; not, as in the present invention, the optimal multi-gene biomarker for specific clinical problem [Simon et al., 2005].

Thus, it is the task of the present invention to make available a test system, with which the rapid and reliable assessment of a pathophysiological condition, such as sepsis and generalized infection, is possible.

With respect to a process, the solution of this task is characterized by the features of claim 1.

With regard to the use, the task is solved by the features of claims 5 to 12.

The solution to the problem involves a primer according to claim 16.

A kit according to claim 17 also solves the problem.

In general terms, the present invention concerns a system that includes the following elements:
a set of gene markers
reference genes as internal control of gene activity marker signals in whole blood
detection mainly by Real-Time PCR or other amplification or hybridization techniques
use of an algorithm to convert the individual results of the marker gene activity into a common numeric value, index or score
representation of this numeric value on an appropriately graded scale
calibration, i.e., dividing up the scale according to the intended application on the basis of previous validation experiments.

The system provides a solution to the problem of determination of disease conditions such as the distinction between infectious and non-infectious multiorgan failure, but also for other applications and objects relevant in this context.

In particular, the present invention concerns a method for in vitro detection and/or early detection and/or differentiation and/or progress monitoring and/or evaluation of pathophysiological conditions, selected from the group consisting of: SIRS, sepsis and its degrees of severity; sepsis like conditions; septic shock; bacteremia, infectious/non-infectious multiorgan failure; early detection of these conditions; focus control; control of surgical rehabilitation of the infection focus; responders/non-responders to a particular therapy; treatment monitoring; distinction between infectious and non-infectious etiology of systemic reactions of the organism, such as e.g. SIRS, sepsis, postoperative complications, chronic and/or acute organ malfunction, shock response, inflammatory response and/or trauma; wherein the method comprises the following steps:
a) isolation of sample nucleic acids from a sample derived from a patient;
b) determination of gene activity by a plurality of at least three polynucleotides selected from the group consisting of M2, M3, M4, M6, M7, M8, M9, M10, M12, M13, M15, M16 and M17, and/or their isoforms and/or their gene loci and/or their transcripts and/or fragments thereof with a length of at least five nucleotides, for establishing at least one characteristic multi-gene biomarker for recording and/or differentiation and/or the progression of pathophysiological conditions of a patient, wherein the polynucleotides are defined according to the following table:

| | Transcript variant/cis- | Accession | SEQ |
|---|---|---|---|
| Polynucleotide | regulatory Sequences | Number | ID NO: |
| M2 | M2_1 | NM_001031700 | 1 |
| | M2_2 | NM_016613 | 2 |
| | M2_3 | NM_001128424 | 3 |
| M4 | M4_1 | NM_203330 | 4 |
| | M4_2 | NM_000611 | 5 |
| | M4_3 | NM_203329 | 6 |
| | M4_4 | NM_203331 | 7 |
| | M4_5 | NM_001127223 | 8 |
| | M4_6 | NM_001127225 | 9 |
| | M4_7 | NM_001127226 | 10 |
| | M4_8 | NM_001127227 | 11 |
| M6 | M6_1 | NM_001831 | 12 |
| | M6_2 | NM_203339 | 13 |
| M7 | M7_1 | NM_031311 | 14 |
| | M7_2 | NM_019029 | 15 |
| M9 | M9 | NM_006682 | 16 |
| M10 | M10 | NM_033554 | 17 |
| M15 | M15_1 | NM_003580 | 18 |
| | M15_2 | NM_001144772 | 19 |
| M3 | M3_A | NM_001123041 | 20 |
| | M3_B | NM_001123396 | 21 |
| M8 | M8 | NM_025209 | 22 |
| | M8_cis | AI807985 | 23 |
| M12 | M12 | NM_002185 | 24 |
| | M12_cis | DB155561 | 25 |
| M13 | M13 | NM_001080394 | 26 |
| M16 | M16 | NM_003268 | 27 |
| M17 | M17 | NM_182491 | 28 |

c) determination of gene activity of at least one internal reference gene to which the gene activities under b) can be referenced, in particular normalized;
d) forming a value from the individually determined gene activities of the multi-gene biomarker, which indicates the pathophysiological condition.

A preferred method is characterized in that the reference gene is selected from polynucleotides of the group consisting of R1, R2 and R3 and/or their isoforms and/or their gene loci and/or their transcripts and/or fragments thereof with a length of at least 5 nucleotides, wherein the reference genes are defined according to the following table:

| | Transcript variants/ | | |
|---|---|---|---|
| Reference | cis-regulatory | Accession | SEQ ID |
| Gene | sequences | Number | NO: |
| R1 | R1_A | NM_001228 | 29 |
| | R1_B | NM_033355 | 30 |
| | R1_C | NM_033356 | 31 |
| | R1_E | NM_033358 | 32 |
| | R1_F | NM_001080124 | 33 |
| | R1_G | NM_001080125 | 34 |
| R2 | R2_1 | NM_002209 | 35 |
| | R2_2 | NM_001114380 | 36 |
| R3 | R3 | NM_003082 | 37 |

A further preferred method is characterized in that as the polynucleotide sequences gene loci, sense, and/or the antisense strands of pre-mRNA and/or mRNA, small RNA, especially scRNA, snoRNA, micro RNA, siRNA, dsRNA, ncRNAs or transposable elements are used to gather the gene expression profiles.

A further preferred embodiment is a process which is characterized in that in step b) the gene activity of 4, 5, 6, 7, 8, 9, 10, 11, or 12 polynucleotides, or of all 13 polynucleotides, is determined, wherein the polynucleotides are selected from the group consisting of: M2, M3, M4, M6, M7, M8, M9, M10, M12, M13, M15, M16 and M17 and/or their isoforms and/or their gene loci and/or their transcripts and/or fragments thereof with a length of at least five nucleotides, wherein the polynucleotides are defined in accordance with the following table:

| | Transcript variants/cis- | Accession | SEQ ID |
|---|---|---|---|
| Polynucleotide | regulatory sequences | Number | NO: |
| M2 | M2_1 | NM_001031700 | 1 |
| | M2_2 | NM_016613 | 2 |
| | M2_3 | NM_001128424 | 3 |
| M4 | M4_1 | NM_203330 | 4 |
| | M4_2 | NM_000611 | 5 |
| | M4_3 | NM_203329 | 6 |
| | M4_4 | NM_203331 | 7 |
| | M4_5 | NM_001127223 | 8 |
| | M4_6 | NM_001127225 | 9 |
| | M4_7 | NM_001127226 | 10 |
| | M4_8 | NM_001127227 | 11 |
| M6 | M6_1 | NM_001831 | 12 |
| | M6_2 | NM_203339 | 13 |
| M7 | M7_1 | NM_031311 | 14 |
| | M7_2 | NM_019029 | 15 |
| M9 | M9 | NM_006682 | 16 |
| M10 | M10 | NM_033554 | 17 |
| M15 | M15_1 | NM_003580 | 18 |
| | M15_2 | NM_001144772 | 19 |
| M3 | M3_A | NM_001123041 | 20 |
| | M3_B | NM_001123396 | 21 |
| M8 | M8 | NM_025209 | 22 |
| | M8_cis | AI807985 | 23 |
| M12 | M12 | NM_002185 | 24 |
| | M12_cis | DB155561 | 25 |
| M13 | M13 | NM_001080394 | 26 |
| M16 | M16 | NM_003268 | 27 |
| M17 | M17 | NM_182491 | 28 |

It has been found that the use of 7 polynucleotides often is optimal.

The invention relates in a further embodiment, in which at least three polynucleotides selected from the group consisting of: M2, M3, M4, M6, M7, M8, M9, M10, M12, M13, M15, M16 and M17 and/or their isoforms and/or their gene loci and/or their transcripts and/or fragments thereof with a length of at least five nucleotides, are used for forming at least one multi-gene biomarkers for producing a multiplex assay as a tool for in vitro detection and/or early detection and/or differentiation and/or progress monitoring and/or evaluation of pathophysiological conditions of a patient, wherein the pathophysiological condition is selected the group consisting of: SIRS, sepsis and its degrees of severity; sepsis-like conditions; septic shock; bacteremia, infectious/non-infectious multiorgan failure; early detection of these conditions; focus control; control of surgical rehabilitation of the infection focus; responders/non-responders to a particular therapy; treatment monitoring; distinction between infectious and non-infectious etiology of systemic reactions of the organism, such as SIRS, sepsis, postoperative complications, chronic and/or acute organ dysfunction, shock response, inflammatory response and/or trauma; wherein the polynucleotides are defined according to the following table:

| | Transcript variants/cis- | Accession | SEQ ID |
|---|---|---|---|
| Polynucleotide | regulatory sequences | Number | NO: |
| M2 | M2_1 | NM_001031700 | 1 |
| | M2_2 | NM_016613 | 2 |
| | M2_3 | NM_001128424 | 3 |
| M4 | M4_1 | NM_203330 | 4 |
| | M4_2 | NM_000611 | 5 |
| | M4_3 | NM_203329 | 6 |
| | M4_4 | NM_203331 | 7 |
| | M4_5 | NM_001127223 | 8 |
| | M4_6 | NM_001127225 | 9 |
| | M4_7 | NM_001127226 | 10 |
| | M4_8 | NM_001127227 | 11 |
| M6 | M6_1 | NM_001831 | 12 |
| | M6_2 | NM_203339 | 13 |
| M7 | M7_1 | NM_031311 | 14 |
| | M7_2 | NM_019029 | 15 |
| M9 | M9 | NM_006682 | 16 |
| M10 | M10 | NM_033554 | 17 |
| M15 | M15_1 | NM_003580 | 18 |
| | M15_2 | NM_001144772 | 19 |
| M3 | M3_A | NM_001123041 | 20 |
| | M3_B | NM_001123396 | 21 |
| M8 | M8 | NM_025209 | 22 |
| | M8_cis | AI807985 | 23 |
| M12 | M12 | NM_002185 | 24 |
| | M12_cis | DB155561 | 25 |
| M13 | M13 | NM_001080394 | 26 |
| M16 | M16 | NM_003268 | 27 |
| M17 | M17 | NM_182491 | 28 |

A preferred embodiment of the present invention is an application in which the multi-gene biomarker is a combination of several polynucleotide sequences, in particular gene sequences, on the basis of which gene activities a classification and/or an index is formed using an interpretation function.

In the practical experiences of the applicant it has been found that a use is particularly suitable which is characterized in that the gene activity was determined by enzymatic methods, in particular amplification techniques, preferably polymerase chain reaction (PCR), preferably real-time PCR, especially probe based procedures such as Taq Man, Scorpions, Molecular beacons, and/or by hybridization procedures, especially those on microarrays; and/or direct mRNA detection, in particular sequencing or mass spectrometry; and/or isothermal amplification.

A further preferred embodiment of the present invention is an application, wherein from the individual gene activities an index made up, which following appropriate calibration is a measure of the severity and/or the course of the pathophysiological condition, where preferably the index is displayed on an easily interpretable scale.

It is also preferred that the obtained gene activity data is used for the production of software for the description of at least one pathophysiologic condition and/or a research issue and/or as a tool for diagnostic purposes and/or patient data management system, particularly for use in the classification of patients, and as an inclusion criterion for clinical trials.

In addition, an application is preferred, in which to create the gene activity data such specific loci, sense and/or antisense strands of pre-mRNA and/or mRNA, small RNA, especially scRNA, snoRNA, micro RNA, siRNA, dsRNA, ncRNAs or transposable elements, genes and/or gene fragments with a length of at least five nucleotides are used that have a sequence homology of at least about 10%, especially about 20%, preferably about 50%, more preferably about 80% to the polynucleotide sequences of M2, M3, M4, M6, M7, M8, M9, M10, M12, M13, M15, M16 and M17.

A further preferred embodiment of the present invention is an application which is characterized in that 4, 5, 6, 7, 8, 9, 10, 11 or 12 polynucleotides, or all 13 polynucleotides, are used, where the polynucleotides are selected from the group consisting of: M2, M3, M4, M6, M7, M8, M9, M10, M12, M13, M15, M16 and M17 and/or their isoforms and/or their gene loci and/or their transcripts and/or fragments thereof with a length of at least five nucleotides, wherein the polynucleotides are defined according to the following table:

| | Transcript variants/cis- | Accession | SEQ ID |
|---|---|---|---|
| Polynucleotide | regulatory sequences | Number | NO: |
| M2 | M2_1 | NM_001031700 | 1 |
| | M2_2 | NM_016613 | 2 |
| | M2_3 | NM_001128424 | 3 |
| M4 | M4_1 | NM_203330 | 4 |
| | M4_2 | NM_000611 | 5 |
| | M4_3 | NM_203329 | 6 |
| | M4_4 | NM_203331 | 7 |
| | M4_5 | NM_001127223 | 8 |
| | M4_6 | NM_001127225 | 9 |
| | M4_7 | NM_001127226 | 10 |
| | M4_8 | NM_001127227 | 11 |
| M6 | M6_1 | NM_001831 | 12 |
| | M6_2 | NM_203339 | 13 |
| M7 | M7_1 | NM_031311 | 14 |
| | M7_2 | NM_019029 | 15 |
| M9 | M9 | NM_006682 | 16 |
| M10 | M10 | NM_033554 | 17 |
| M15 | M15_1 | NM_003580 | 18 |
| | M15_2 | NM_001144772 | 19 |
| M3 | M3_A | NM_001123041 | 20 |
| | M3_B | NM_001123396 | 21 |
| M8 | M8 | NM_025209 | 22 |
| | M8_cis | AI807985 | 23 |
| M12 | M12 | NM_002185 | 24 |
| | M12_cis | DB155561 | 25 |
| M13 | M13 | NM_001080394 | 26 |
| M16 | M16 | NM_003268 | 27 |
| M17 | M17 | NM_182491 | 28 |

and/or wherein a number of polynucleotides preferably is 7.

Basically, the invention can also be executed according to an alternative embodiment using at least one polynucleotide selected from the group consisting of: M2, M3, M4, M6, M7, M8, M9, M10, M12, M13, M15, M16 and M17 and/or their isoforms and/or their gene loci and/or their transcripts and/or fragments thereof with a length of at least five nucleotides, wherein the polynucleotides are defined according to the following table:

| | Transcript variants/cis- | Accession | SEQ ID |
|---|---|---|---|
| Polynucleotide | regulatory sequences | Number | NO: |
| M2 | M2_1 | NM_001031700 | 1 |
| | M2_2 | NM_016613 | 2 |
| | M2_3 | NM_001128424 | 3 |
| M4 | M4_1 | NM_203330 | 4 |
| | M4_2 | NM_000611 | 5 |
| | M4_3 | NM_203329 | 6 |
| | M4_4 | NM_203331 | 7 |
| | M4_5 | NM_001127223 | 8 |
| | M4_6 | NM_001127225 | 9 |
| | M4_7 | NM_001127226 | 10 |
| | M4_8 | NM_001127227 | 11 |
| M6 | M6_1 | NM_001831 | 12 |
| | M6_2 | NM_203339 | 13 |
| M7 | M7_1 | NM_031311 | 14 |
| | M7_2 | NM_019029 | 15 |
| M9 | M9 | NM_006682 | 16 |
| M10 | M10 | NM_033554 | 17 |
| M15 | M15_1 | NM_003580 | 18 |
| | M15_2 | NM_001144772 | 19 |
| M3 | M3_A | NM_001123041 | 20 |
| | M3_B | NM_001123396 | 21 |
| M8 | M8 | NM_025209 | 22 |
| | M8_cis | AI807985 | 23 |
| M12 | M12 | NM_002185 | 24 |
| | M12_cis | DB155561 | 25 |
| M13 | M13 | NM_001080394 | 26 |
| M16 | M16 | NM_003268 | 27 |
| M17 | M17 | NM_182491 | 28 |

for the production of an assay for determining whether a patient is presenting a pathophysiological condition, and/or to determine the severity and/or the progression of a pathophysiological condition.

Herein the pathophysiological condition is selected from the group consisting of: SIRS, sepsis and its degrees of severity; sepsis-like conditions; septic shock; bacteremia; infectious/non-infectious multiorgan failure; early detection of these conditions; focus control; control of surgical rehabilitation of the infection focus; responder/non-responders to a particular therapy; treatment monitoring; distinction between infectious and non-infectious etiology of systemic reactions of the organism, such as SIRS, sepsis, postoperative complications, chronic and/or acute organ dysfunction, shock response, inflammatory response and/or trauma.

Preferably, the sample nucleic acid is RNA, in particular, whole RNA or mRNA, or DNA, especially cDNA.

For a more refined diagnostic information, it can be of advantage in the assessment of the pathophysiological condition to use, in addition to the at least one of the polynucleotides, selected the group consisting of M2, M3, M4 M6, M7, M8, M9, M10, M12, M13, M15, M16 and M17 and/or their isoforms and/or their gene loci and/or their transcripts and/or fragments thereof with a length of at least five nucleotides, wherein the polynucleotides are defined according to following table:

| | Transcript variants/cis- | Accession | SEQ ID |
|---|---|---|---|
| Polynucleotide | regulatory sequences | Number | NO: |
| M2 | M2_1 | NM_001031700 | 1 |
| | M2_2 | NM_016613 | 2 |
| | M2_3 | NM_001128424 | 3 |
| M4 | M4_1 | NM_203330 | 4 |
| | M4_2 | NM_000611 | 5 |
| | M4_3 | NM_203329 | 6 |
| | M4_4 | NM_203331 | 7 |
| | M4_5 | NM_001127223 | 8 |
| | M4_6 | NM_001127225 | 9 |
| | M4_7 | NM_001127226 | 10 |
| | M4_8 | NM_001127227 | 11 |
| M6 | M6_1 | NM_001831 | 12 |
| | M6_2 | NM_203339 | 13 |
| M7 | M7_1 | NM_031311 | 14 |
| | M7_2 | NM_019029 | 15 |
| M9 | M9 | NM_006682 | 16 |
| M10 | M10 | NM_033554 | 17 |
| M15 | M15_1 | NM_003580 | 18 |
| | M15_2 | NM_001144772 | 19 |
| M3 | M3_A | NM_001123041 | 20 |
| | M3_B | NM_001123396 | 21 |
| M8 | M8 | NM_025209 | 22 |
| | M8_cis | AI807985 | 23 |
| M12 | M12 | NM_002185 | 24 |
| | M12_cis | DB155561 | 25 |
| M13 | M13 | NM_001080394 | 26 |
| M16 | M16 | NM_003268 | 27 |
| M17 | M17 | NM_182491 | 28 |

to additionally use at least another marker, which is selected from the group consisting of: procalcitonin (PCT), C-reactive protein (CRP), leukocyte count, cytokines, interleukins and other prior art clinical laboratory parameters and genetic, transcriptomic and proteomic markers well-known to the person of ordinary skill in the art.

In order to carry out the present invention, it is necessary to employ suitable primer pairs (forward and reverse). Particularly suitable primers of this type are those which are enumerated in the following table:

| Markers and | | |
|---|---|---|
| Reference | Primers for quantitative | SEQ |
| Genes | PCR/resulting amplicon | ID NO: |
| M2 | M2-fw | 38 |
| | M2-rev | 39 |
| | M2-Amplikon | 40 |
| M4 | M4-fw | 41 |
| | M4-rev | 42 |
| | M4-Amplikon | 43 |
| M6 | M6-fw | 44 |
| | M6-rev | 45 |
| | M6-Amplikon | 46 |
| M7 | M7-fw | 47 |
| | M7-rev | 48 |
| | M7-Amplikon | 49 |
| M9 | M9-fw | 50 |
| | M9-rev | 51 |
| | M9-Amplikon | 52 |
| M10 | M10-fw | 53 |
| | M10-rev | 54 |
| | M10-Amplikon | 55 |
| M15 | M15-fw | 56 |
| | M15-rev | 57 |
| | M15-Amplikon | 58 |
| M3 | M3-fw | 59 |
| | M3-rev | 60 |
| | M3-Amplikon | 61 |
| M8 | M8-fw | 62 |
| | M8-rev | 63 |
| | M8-Amplikon | 64 |
| M12 | M12-fw | 65 |
| | M12-rev | 66 |
| | M12-Amplikon | 67 |
| M13 | M13-fw | 68 |
| | M13-rev | 69 |
| | M13-Amplikon | 70 |
| M16 | M16-fw | 71 |
| | M16-rev | 72 |
| | M16-Amplikon | 73 |
| M17 | M17-fw | 74 |
| | M17-rev | 75 |
| | M17-Amplikon | 76 |
| R1 | R1-fw | 77 |
| | R1-rev | 78 |
| | R1-Amplikon | 79 |
| R2 | R2-fw | 80 |
| | R2-rev | 81 |
| | R2-Amplikon | 82 |
| R3 | R3-fw | 83 |
| | R3-rev | 84 |
| | R3-Amplikon | 85 |

However, it must be stressed that these primers are merely illustrative.

The above exemplified Amplicons can be used, for example, as probes in hybridization techniques.

The invention also relates to a kit for carrying out the invention, containing at least one multi-gene biomarker, which comprises a plurality of polynucleotide sequences, which are selected from the group consisting of: M2, M3, M4, M6, M7, M8, M9, M10, M12, M13, M15, M16 and M17 and/or their isoforms and/or their gene loci and/or their transcripts and/or fragments thereof with a length of at least five nucleotides, wherein the polynucleotides are defined according to the following table:

| Markers | | | |
|---|---|---|---|
| and | | | |
| Reference | Transcript variants/cis- | Accession | SEQ ID |
| Genes | regulatory sequences | Number | NO: |
| M2 | M2_1 | NM_001031700 | 1 |
| | M2_2 | NM_016613 | 2 |
| | M2_3 | NM_001128424 | 3 |
| M4 | M4_1 | NM_203330 | 4 |
| | M4_2 | NM_000611 | 5 |
| | M4_3 | NM_203329 | 6 |
| | M4_4 | NM_203331 | 7 |
| | M4_5 | NM_001127223 | 8 |
| | M4_6 | NM_001127225 | 9 |
| | M4_7 | NM_001127226 | 10 |
| | M4_8 | NM_001127227 | 11 |
| M6 | M6_1 | NM_001831 | 12 |
| | M6_2 | NM_203339 | 13 |
| M7 | M7_1 | NM_031311 | 14 |
| | M7_2 | NM_019029 | 15 |
| M9 | M9 | NM_006682 | 16 |
| M10 | M10 | NM_033554 | 17 |
| M15 | M15_1 | NM_003580 | 18 |
| | M15_2 | NM_001144772 | 19 |
| M3 | M3_A | NM_001123041 | 20 |
| | M3_B | NM_001123396 | 21 |
| M8 | M8 | NM_025209 | 22 |
| | M8_cis | AI807985 | 23 |
| M12 | M12 | NM_002185 | 24 |
| | M12_cis | DB155561 | 25 |
| M13 | M13 | NM_001080394 | 26 |
| M16 | M16 | NM_003268 | 27 |
| M17 | M17 | NM_182491 | 28 |

wherein the multi-gene biomarker is specific to a pathophysiological condition of a patient and that includes conditions which are selected the group consisting of: SIRS, sepsis and its degrees of severity; sepsis-like conditions; septic shock; bacteremia; infectious/non-infectious multiorgan failure; early detection of these conditions; focus control; control of surgical rehabilitation of the infection focus; responders/non-responders to a particular therapy; treatment monitoring; distinction between infectious and non-infectious etiology of systemic reactions of the organism, such as SIRS, sepsis, postoperative complications, chronic and/or acute organ dysfunction, shock response, inflammatory response and/or trauma.

A preferred kit is characterized in that the polynucleotide sequences also include gene loci, sense and/or antisense strands of pre-mRNA and/or mRNA, small RNA, especially scRNA, snoRNA, micro RNA, siRNA, dsRNA, ncRNA or transposable elements.

A further preferred kit is characterized in that it contains at least one reference gene that is selected from the group consisting of: R1, R2 and R3 and/or their isoforms and/or their gene loci and/or their transcripts and/or fragments thereof with a length of at least five nucleotides, wherein the reference genes are defined according to the following table:

| | Transcript variants/ | | |
|---|---|---|---|
| Reference | cis-regulatory | Accession | SEQ ID |
| Gene | sequences | Number | NO: |
| R1 | R1_A | NM_001228 | 29 |
| | R1_B | NM_033355 | 30 |
| | R1_C | NM_033356 | 31 |
| | R1_E | NM_033358 | 32 |
| | R1_F | NM_001080124 | 33 |
| | R1_G | NM_001080125 | 34 |
| R2 | R2_1 | NM_002209 | 35 |
| | R2_2 | NM_001114380 | 36 |
| R3 | R3 | NM_003082 | 37 |

A likewise preferred application is characterized in that from the individually determined gene activities an index (score) is formed, which after appropriate calibration represents a value or measure of the severity and/or the course of the pathophysiological condition, particularly sepsis or the sepsis-like condition.

It is also preferred to display the index (score) on an easily interpretable scale.

In practical experiences of the applicant it has been found that a dimensionless scale of −5 to +5, or, to enhance the differences using an appropriate multiple, e.g. from −50 to +50, is particularly suited to classify pathophysiological conditions. Dieser Score wird “SIQ-Score” genannt. This score is referred to as the “SIQ Score”.

In the framework of an optimized computer-based hospital management, as well as for further research in the field of sepsis, it has proved beneficial to use the obtained gene activity data for the production of software for the description of at least one pathophysiologic condition and/or a research issue and/or tools for diagnostic purposes and/or patient data management systems.

The index is preferably developed by means of statistical methods such as supervised classification methods from the field of mechanical and statistical learning such as (diagonal, linear, quadratic) discriminant analysis, super vector machines, generalized partial least squares, k-nearest neighbors, random forests, k-nearest neighbor. For example, for a linear discriminant the following formula may be used:

$${f}_{LD} ( {x}_{1} , … , {x}_{p} ) = {\sum }_{i = 1}^{p} {w}_{i} {x}_{i} - {w}_{0}$$

The multi-gene biomarker is preferably a combination of several polynucleotide sequences, particularly gene sequences, on the basis of the genetic activity of which, using an interpretation function, a classification is carried out and/or an index or score is developed.

For the purposes of the present invention, it has proved to be of further advantage, that the gene activity is determined using enzymatic methods, in particular amplification technique, preferably polymerase chain reaction (PCR), preferably real-time PCR, and/or by hybridization methods, particularly those on microarrays.

Differential expression signals of the polynucleotide sequences contained in multi-gene biomarker occurring during the gathering of the gene activity can be beneficial and clearly linked to or associated with a pathophysiological condition, a course and/or treatment monitoring.

Typically, from the individual gene activities an index (score, SIQ-score) is formed, which, after appropriate calibration, is a measure of the severity and/or the course of the pathophysiological condition, particularly sepsis or the sepsis-like condition.

This score can put a rapid diagnostic tool in the hands of the doctor.

The present invention makes it possible, as part of an integrated system (“In Vitro Diagnostic Multivariate Index Assay” (IVDMIA)) to assess a potential infectious complication in patients with SIRS or possible sepsis. This system involves the selection of patients and determination of their gene expression signals in an interpretable index which the physician can use as an aid to diagnosis.

The applicant has developed several methods, which use different sequence pools, to detect conditions and/or to distinguish or to answer research issues. Examples can be found in the following patents: the distinction between SIRS, sepsis, and sepsis-like conditions [WO 2004/087949, WO 2005/083115], establishment of criteria for predicting disease progression in sepsis [WO 05/106020], differentiation between infectious and noninfectious causes a multiorgan failure [WO 2006/042581], in vitro classification of gene expression profiles of patients with infectious/noninfectious multiorgan failure [WO 2006/100203], detection of the local causes of fever of unknown origin [WO 2007/144105], polynucleotides for the detection of gene activity for the distinction between local and systemic infection [DE 10 2007 036 678.9].

The invention relates to polynucleotide sequences, a process, and also kits for creating multi-gene biomarkers, which in one and/or multi-modules exhibit features of an “In Vitro Diagnostic Multivariate Index Assay” (IVDMIA).

Regarding the nucleotide sequences used in the present application, the following is to be noted:

RefSeq is a public database which includes information of nucleotide and protein sequences with their properties as well as bibliographic information.

The RefSeq database was established by the National Center for Biotechnology Information (NCBI), a division of National Library of Medicine and the U.S. National Institutes of Health and is maintained and updated continuously (1).

NCBI creates RefSeq from the sequence data of the archive database “GenBank” (2), a comprehensive public database of sequences in GenBank in the U.S., the EMBL data library in the UK, and the DNA Database of Japan and also data exchanged between these databases.

The RefSeq collection is unique with regard to the provision of error-corrected non-redundant, explicitly linked nucleotide and protein databases. The entries are non-redundant with the aim to represent the different biological molecules, which are characteristic for the organism, strain or haplotype.

If certain items in the collection occur multiple times, there may be several reasons for this:
alternative spliced transcripts encode for the same protein product (known transcript variants)
there are several genomic regions within a species or between species, which encode the same protein product,
when RefSeqs are created, which represent the alternative haplotypes present, and some of mRNA and protein sequences are identical in all haplotypes.

RefSeq database provides the critical foundation for integrating sequence, genetic and functional information and is regarded internationally as the standard for genome annotation. In a sequence search using BLAST the RefSeq details in several NCBI resources are available, including Entrez Nucleotide, Entrez Protein, Entrez Gene, Map Viewer, the FTP download, or by networking with PubMed (Pruitt et al. 2007; The NCBI handbook 2002). RefSeq Accession information may be identified by the unique format, which includes the underscore (_).

Working groups use various methods and protocols, and compile the RefSeq collection for different organisms. RefSeq records are created by several different methods (The NCBI Handbook 2002):
1. scientific cooperation
2. computer-assisted genome annotation processes
3. error correction by the NCBI staff
4. extracts from GenBank

Each item of data has a comment that has the status of the various error corrections as well as the association of the working group. Thereby the RefSeq data is either the oldest running RefSeq which is an essentially unchanged initially valid copy of the original GenBank entries, or a corrected version with additional information added by cooperation partners or person of ordinary skill in the arts (The NCBI Handbook 2002).

If a molecule is represented by several sequences in GenBank, the NCBI staff make a decision as to the “best” sequence, and this is then presented in RefSeq.

The main objective is the avoidance of known mutations, sequencing errors, cloning artifacts, and erroneous annotations. RefSeq sequences which are afflicted with these types of errors will be corrected. Sequences are validated by checking whether the genomic sequence, which corresponds to the annotated mRNA, actually fits for the mRNA sequence, and whether coding regions are actually translated into the corresponding protein sequence. Another important task is to improve the collection by adding previously unknown underrepresented genes and/or alternative splice products, as well as additional of annotation of sequence features which represent mature peptide products and their functional domains and/or biological phenomena, such as, e.g., non-AUG initiation sites of transcription or selenoproteins (The NCBI Handbook 2002).

The review of the quality occurs on a regular basis, to check for and find questionable sequences. These quality tests check the errors and conflicts in nomenclature, sequence similarities and genomic localization, potential cloning errors (e.g., chimeras) and compare the data with other NCBI resources, including HomoloGene, Map Viewer and the GenBank related sequences from (The NCBI handbook 2002).

With the present high-qualitative genomic sequences of human and mouse, the checking of cDNA based RefSeqs in relation to the genome was the main focus. The CODS Cooperation (The NCBI handbook 2002) has also helped to focus attention on areas where discrepancies existed between mRNA and protein quantity.

Quality assurance processes includes the registration of database attributes, in order to document that
the category of quality testing has been updated
no problems with the RefSeq transcript and protein were found and therefore the reported errors should be ignored,
a problem at this position was determined using genome assembly
   there could be problems of genome assembly
   there are gaps in the joining of individual sequences
      in some cases the established sequence includes a known mutation or rare polymorphism and is therefore not an ideal representative sequence (Pruitt et al. 2007).

The decision to use, in the present application, known marker populations on the basis of their RefSeq identity for the purposes of the present invention was arrived at as a result of the above-described properties of the RefSeq database. The characteristics of this database, the production, quality, care and updates on biological sequences, and the existence of functional information on the nucleic acid level, as well as for alternative splice variants, was the decisive factor.

As explained above, the biological mechanism of alternative splicing provides flexibility for the person of ordinary skill in this art to extend the scope of protection. Thus, it is conceivable that with new transcript variants completely new primary structures will be identified, or that sequence changes will occur in the known transcript variants. On the other hand, those genomic regions are claimed, that encompass for all these known and unknown variants of coding transcripts, including their cis-regulatory sequences as complete genomic functional units and thus fall within the scope of the present invention, or at least put within the reach of the person of ordinary skill in the art easily obtainable equivalents to those sequences recited in the claims, specification and sequence listing.

### DEFINITIONS

For the purposes of the present invention the following definitions are used:

Condition: the clinically defined severity “systemic inflammatory response syndrome” (SIRS), “sepsis,” “severe sepsis” and “septic shock” as defined in [Bone et al., 1992] and the PIRO concept [Levy et al. 2003].
Multiorgan failure: a multiorgan failure is defined as the simultaneous or in rapid succession occurring failure of two or more vital organ systems. The multiorgan dysfunction syndrome (MODS) precedes the initial organ failure of the MOV [Zeni et al., 1997]. One speaks today of multiorgan failure when two or more organs have functional disorders simultaneously or in succession, excluding chronic persistent organ failure. The forecast of the MOV is closely associated with the number of organ systems involved. The mortality rate is 22% within the first 24 hours in case of failure of one organ, 41% after 7 days. In the case of failure of three organ systems mortality rises to 80% on the first day and after 4 days it is at 100% to [Knaus et al., 1985].

An important pathogenic mechanism for the development of MODS and MOV is the development of Systemic Inflammation Syndrome (SIRS), [Bone et al., 1992]. MODS and MOV can be caused by host resistance to infectious as well as non-infectious diseases.

Fever of unknown origin: a fever of unknown origin (Fever of Unknown Origin, FUO) is defined clinically as a fever, in which the temperature remains above 38.8° C. for more than three weeks and no clear diagnosis of the cause is present after a week-long examination time. Depending on the origin of FUO four classes were described: classic FUO, nosocomial, immunocompromised and HIV-related origin [Roth and Basello, 2003]. FUO was also called “a rather well-known disease with an unusual appearance as a rare disorder” described [Amin and Kauffman, 2003].

Infection is a documented in only 10% of patients with postoperative fever [Pile et., 2006]. In most cases the temperature of the patient is back to normal within four days after the operation. Nevertheless, some patients develop an infection on or after the fifth postoperative day, it is pneumonia in 12% of cases. Similarly, Pile and colleagues reported that in the case of fever, which appears two days after the procedure, it is most likely an infection, such as a urinary tract infection and/or an infection of the inner abdomen (peritonitis), pneumonia or an infection induced by an intravenous catheter.

Investigation issue: a clinically relevant issue which is of importance for the treatment of a patient, for example: prediction of disease progression, treatment monitoring, focus of infection, survival, predisposition, etc.

A systemic infection is an infection in which the pathogens have spread via the bloodstream throughout the body.

SIRS: Systemic Inflammatory Response Syndrome according to Bone [Bone et al., 1992] and Levy [Levy et al., 2003] is a generalized, inflammatory, noninfectious condition of a patient.
Sepsis: according to Bone [Bone et al., 1992] and Levy [Levy et al., 2003] this is a generalized, infectious inflammatory condition of a patient.
Biological fluid: biological fluid, in the context of the invention, refers to all body fluids of mammals, including humans.
Gene: a gene is a segment of deoxyribonucleic acid (DNA), which contains the basic information for making a biologically active ribonucleic acid (RNA) as well as regulatory elements that activate or inactivate such manufacture. As genes in the context of the invention, all derived DNA sequences, partial sequences and synthetic analogs (for example petidonucleic acids (PNA)) are understood. The identification of the gene expression being at the RNA level in the description of the invention is not an explicit restriction but only an exemplary application.
Gene locus: (locus) is the position of a gene in the genome. If the genome comprises several chromosomes, the position is meant within the chromosome that contains the gene. Different forms or variants of this gene are called alleles, which are all in the same location on the chromosome, namely the locus. Thus, the term “locus” includes on the one hand the pure genetic information for a specific gene product and on the other hand all other regulatory DNA segments as well as any additional DNA sequences, which are related to the gene at the locus in any functional relationship. The latter attach to sequence regions in the immediate vicinity (1 Kb) but are located outside of the 5-′ and/or 3′ end of a gene locus. The specifying of the locus is done by the Accession number and/or RefSeq ID of the RNA main product, which is derived from this locus.
Gene activity: gene activity is the magnitude and the ability of a gene to be understood, transcribed and/or to produce translation products.
Gene expression: The process of forming a gene product and/or expression of a genotype into a phenotype.
Multi-gene biomarker: a combination of several gene sequences, the gene activities of which, using an interpretation function, produce a combined total (eg, a classification and/or index form). This result is specific to one condition and/or a research issue.
Hybridization conditions: The physical and chemical parameters well-known to the person of ordinary skill, which may influence the establishment of a thermodynamic equilibrium of free and bound molecules. In the interest of optimal hybridization conditions the duration of contact of the probe and sample molecules, the cation concentration in the hybridization buffer, temperature, volume as well as concentrations and relationship of the hybridizing molecules must be coordinated.
Amplification conditions: Constant or cyclically changing reaction conditions which allow the multiplication of the starting material in the form of nucleic acids. The reaction mixture includes the individual building blocks (deoxyribonucleotides) for the resulting nucleic acids, as well as short oligonucleotides, which may attach to complementary areas in the source material, and a nucleic acid synthesis enzyme, called a polymerase. The person of ordinary skill is aware of the cation concentrations, pH, volume and duration and temperature of individual reaction steps that are of importance in the progress of amplification.
Primer: in the present invention the primer is an oligonucleotide, which serves as a starting point for nucleic acid replicating enzymes such as DNA polymerase. Primers could be comprised of DNA as well as RNA (Primer3, see eg http://frodo.wi.mit.edu/cgi-bin/primer3/primer3_www.cgi MIT).
Probe: in the present application, a probe is a nucleic acid fragment (DNA or RNA) that can be coated with a molecular marker (e.g. fluorescent label, especially Scorpion®, molecular beacons, Minor Groove Binding probes, TaqMan® probes, isotope labeling, etc.) and is used for sequence-specific detection of target DNA and/or target RNA molecules.
PCR: is the abbreviation for the English term “Polymerase Chain Reaction” (PCR). The polymerase chain reaction is a method to make multiple copies of DNA in vitro outside a living organism with a DNA-dependent DNA polymerase. PCR is used in accordance with the present invention in particular to reproduce short segments—up to about 3000 base pairs—of a DNA strand of interest. This may be a gene or only a part of a gene or even non-coding DNA sequences. The ordinary technician well knows that a series of PCR methods are known in the art, all of which are encompassed by the term “PCR”. This is particularly true for the “Real-Time PCR” (see also the discussion below).
PCR Primer: PCR typically requires two primers, in order to locate to the start point of DNA synthesis on the two single strands of DNA, wherein the area to be replicated is bounded on both sides. Such primers are well known to the ordinary technician, see the Website “Primer3”, see for example http://frodo.wi.mit.edu/cgi-bin/primer3/primer3_www.cgi from MIT.
Transcript: for the purposes of the present application this is understood to be a transcript of any RNA product that is manufactured using a DNA template.
Small RNA: refers to small RNAs in general. Representatives of this group are particularly, but not exclusively:
a) scRNA (small cytoplasmatic RNA), which is one of several small RNA molecules in the cytoplasm of a eukaryote.
b) snRNA (small nuclear RNA), one of the many small forms of RNA that occur only in the nucleus. Some of the snRNAs play a role in splicing or other RNA processing reactions.
c) small non-protein-coding RNAs, which include the so-called small nucleolar RNAs (snoRNAs), microRNAs (miRNAs), short interfering RNAs (siRNAs) and small double-stranded RNAs (dsRNAs) involved in gene expression at many levels, including chromatin architecture, RNA editing, RNA stability, translation, and possibly also transcription and splicing. In general, these RNAs in multiple ways processed from the introns and exons of longer primary transcript, including protein-coding transcripts. Although approximately only 1.2% of the human genome encodes proteins, a large part is nevertheless transcribed. In fact, approximately 98% of the transcripts found in mammals and humans are from non-protein-coding RNAs (ncRNA), from introns of protein coding genes and exons and introns of non-protein coding genes, including many, which are anti-sense to protein-coding genes or overlap with these. Small nucleolar RNAs (snoRNAs) regulate the sequence-specific modification of nucleotides in target RNAs. Herein there are two types of modifications, 2′-O-ribose methylation and pseudouridylation, which are regulated by two large snoRNA families, which are called, on the one hand, box C/D snoRNAs and, on the other hand, box H/ACA snoRNAs. Such snoRNAs have length of about 60 to 300 nucleotides. miRNAs (microRNAs) and siRNAs (short interfering RNAs) are even smaller RNAs with 21-25 nucleotides. miRNAs come from endogenous short hairpin precursor structures, and usually use other loci with similar—but not identical—sequences as the target of translational repression. siRNAs arise from longer double stranded RNAs or long hairpins, often of exogenous origin. They usually have homologous sequences at the same locus or elsewhere in the genome as the target, where they are involved in gene silencing, a phenomenon which is also called RNAi. The boundaries between miRNAs and siRNAs are, however, blurred.
d) in addition, term “small RNA” can also include the so-called transposable elements (TEs), especially the retro elements, which likewise, for the purposes of the present invention, fall within the meaning of the term “small RNA”.
RefSeq ID: This term refers to entries in the NCBI database (www.ncbi.nlm.nih.gov). This database provides non-redundant reference standards of genomic information. This genomic information includes, inter alia, chromosomes, mRNAs, RNAs and proteins. Each RefSeq ID represents a single, naturally occurring molecule in an organism. The biological sequences, which represent a RefSeq, are derived from GenBank entries (again NCBI), are however a compilation of information elements. These pieces of information come from primary research on DNA, RNA and protein level.
Accession Number: an accession Number is the entry or registration number of a polynucleotide in the NCBI GenBank database which is known to those working in the art. In this data bank RefSeq ID's as well as less well-characterized sequences and redundant entries are used as entries for which access is given to the public (www.ncbi.nlm.nih.gov/Genbank/index.html).
Local infection: the infection is limited to the portal of entry of the pathogen (e.g. wound infection).
Generalized infection: pathogens invade the vascular system and involve the whole body. Generalized infections can lead to sepsis.
Colonization: The presence of micro-organisms in the body absent any disease symptoms whatsoever.
Severe infection: viral focus with the danger of increasing spread with symptoms being fever of 39° C. and above and/or bacteremia.
Bacteremia: A condition in which bacteria are present in the blood short-term and temporary, without necessarily being associated with the occurrence of bacterial clinical symptoms.
Alternative splicing: a process in which the exons of the primary gene transcript (pre-mRNA) are reconnected, after excision of introns, in various combinations.
BLAST: Basic Local Alignment Search Tool (by Altschul et al., J Mol Biol 215:403-410, 1990). Sequence comparison algorithm, speed optimized, used for the search in sequence databases for optimal local conformity to the request sequence.
cDNA: Complementary DNA. DNA sequence, product of reverse transcription of mRNA.
Coding sequence: protein-coding segment of a gene or an mRNA, as distinguished from introns (noncoding sequences) and 5′- or 3′-untranslated segments. Coding sequences of the mature mRNA or cDNA include the area between the start (AUG or ATG) and stop codon.
EST: Expressed Sequence Tag: Short ssDNA segments of cDNA (typically 300-500 bp), usually produced in large quantities. Represent the genes that are expressed in particular tissues and/or during certain stages of development. Partially cCoding or non-coding labels or unique codes of expression for cDNA libraries. Valuable for determining the size of complete genes and in the context of mapping (mapping).
Exon: the sequence region of typical of eukaryotic genes coding information that is transcribed to mRNA. Exons can include the coding sequences, the 5′-untranslated region or the 3′-untranslated region. Exons encode specific sections of the complete protein and are usually interrupted by long segments (introns), which have until now been referred to as “junk DNA” as their function is not precisely known but probably encodes short, non-translated RNAs (snRNA) or regulatory information.
GenBank: nucleotide sequence database with sequences from more than 100,000 organisms. Records, that are annotated with properties the coding regions, also include the translation products. GenBank is part of the international collaboration of sequence databases, including EMBL and DDBJ.
Intron: non-coding sequence region of a typical eukaryotic gene, is excised out of the primary transcript during RNA splicing and thus is not present in the mature, functional mRNA, rRNA or tRNA.
mRNA: messenger RNA, or sometimes only “message”. RNA, die die für Proteinkodierung notwendigen Sequenzen enthält. RNA which contains the sequences necessary for protein coding. The term mRNA is used, in distinction to the (unspliced) primary transcript, to refer only to the mature transcript with polyA-tail (exclusive of the introns removed by splicing). Has 5′-untranslated, amino acid coding-, 3′-untranslated regions and (almost always) a poly(A)-tail. Typically constitutes about 2% of total cellular RNA.
Poly (A) tail: ss adenosine extension (˜50-200 monomers) which extends from the 3′ end of mRNA during splicing. The polyA-tail presumably increases the stability of the mRNA (possibly protection against nucleases). Not all mRNA have this construct, for example, the histone mRNA.
RefSEQ: NCBI-NCBI database of reference sequences. Error-corrected, non-redundant sequence collection of genomic DNA contigs, mRNA sequences and protein sequences in cases of known genes and/or complete chromosomes.
SNPs: Single Nucleotide Polymorphisms: Single Nucleotide Polymorphisms. Genetic differences between alleles of the same gene based on a single nucleotide difference. Emerge at specific individual positions within a gene.
Transcript variants: alternative splicing products. The exons of the primary gene transcript (pre-mRNA) have been reconnected in different ways and are subsequently translated.
3′-untranslated region: transcribed 3′-terminal mRNA area without protein-coding information (area between stop codon and polyA-tail). Could influence the translation efficiency or stability of the mRNA.
5′-untranslated region: transcribed 5′-terminal mRNA area without protein-coding information (area between initial 7-methylguanosine and the base immediately before the ATG start codon). Could influence the translation efficiency or stability of the mRNA.
Polynucleotide isoforms: polynucleotides with the same function but with different sequences.

### Abbreviations

AUC Area under the curve
CRP C-reactive protein
CV cross-validation
DLDA diagonal linear discriminant analysis classification process
GPLS generalized partial least squares (classification method)
IQR (inter quartile range) distance between the 75% and 25% percentile
kNN k-nearest neighbors (classification method)
LDA linear discriminant analysis (classification method)
NPV negative predictive value (proportion of correct negative tests) (classification method)
OR Odd Ratio
PCT Procalcitonin
PPV positive predictive value (proportion of correct negative tests)
RF random forests classification methods
ROC receiver operator characteristics representation of characteristics for classification results
Sensitivity proportion of correct tests in the Group with specified Disease (infectious SIRS or Sepsis)
Specificity amount of correct tests in the group without the specified Disease (non-infectious SIRS)
SVM support vector machines (classification method)

For a rapid diagnosis, it has been found in practice that real-time amplification methods are preferred. For this reason, in the following the basics, which are well known to the person of ordinary skill in this art, will be briefly reviewed with respect to their significance to the present invention.

Other methods well known to the person of ordinary skill in this art are also within the scope of the invention, such as sequencing, micro-array based methods, NASBA, etc.

Using polymerase chain reaction (PCR), it is possible in vitro to rapidly amplify low initial quantities of sequence specific areas of nucleic acids, in order to make them available for further analysis or further processing. A double stranded DNA molecule is denatured by heating. The single strands are used in the sequence as a template for the enzymatically catalyzed polymerization of deoxyribonucleotides, whereby double-stranded DNA molecules are formed again. The oligodeoxyribonucleotides designated as primers define the sequence segment to be copied, in that they hybridize with the target DNA at sites with complementary sequences and serve as initiators for the polymerization. The process of exponential product formation is limited by several factors. In the course of PCR, the net product formation finally goes to zero and the total amount of PCR product reaches a plateau.

Suitable PCR primers include primers with the sequences in Table Appropriate PCR primers include primers with the sequences in Table 3. The person of ordinary skill in the art is however also aware that a variety of other primers can be used for performing the present invention.

Since its introduction in the spectrum of molecular biological methods, an almost unmanageable variety technical options developed by. Today, PCR is one of the most important methods in molecular biology and molecular medicine. Today it is used in a very broad thematic spectrum, such as the detection of viruses or bacteria, in sequencing, the proof of blood relationship (e.g., paternity testing), in preparation of transcription profiles and the quantification of nucleic acids [Valasek and Repa, 2005; Klein, 2002]. Moreover, with the help of PCR in simple manner any of the nucleic acid sequence segments in an organism can be cloned. The large number of developed PCR variants also allows for a targeted or random change in DNA sequence, and even the synthesis of larger, in this form not previously existing, sequence sequences.

With this classical method DNA and, via reverse transcription (RT), also RNA, can be qualitatively measured with high sensitivity [Wong et al., 2005; Bustin 2002]. A further development of this method is Real-Time PCR, which was first introduced in 1991 and besides qualitative statements also makes quantification possible.

Real-time PCR, and quantitative PCR (qPCR) called, is a method for detecting and quantifying nucleic acids in real time [Nolan et al., 2006]. In molecular biology it has for some years to the established standard techniques.

In contrast to the PCR, in the present invention the detection is already taking place during amplification. Based on fluorescence-labeled probes, the fluorophores, the amplification can be followed in real time. In each reaction cycle, there is an increase in fluorescent of PCR products and thus an in crease in intensity of light-induced fluorescence emission. Since the increase in fluorescence and the amount of newly synthesized PCR products are proportional to each other over a wide range, the obtained data can be used to determine the initial quantity of the template. A gel electrophoretic separation of the amplified products is no longer necessary. The results are available directly, which is associated with it a significant time savings. Since the reactions occur in closed vessels, and since no additional pipetting steps are necessary after the start of PCR, the risk of contamination is reduced to a minimum. As fluorophores there may be employed either nucleic acid-binding fluorescent dyes such as SYBRGreen or sequence-specific fluorescent probes such as Taq-Man probes, LightCycler probes and molecular beacons used [Kubista et al., 2006]. SYBRGreen fluorescence is a dye, which increases strongly in fluorescence as soon as the molecule binds to double-stranded DNA. This cost-effective solution is particularly advantageous with the implementation of several parallel reactions with different primer pairs. Disadvantages are the low specificity, since SYBRGreen binds sequence-specific to any double-stranded DNA, and further therein, that no multiplex measurements can be performed. With the aid of a decomposition curve analysis, after successful PCR, differentiation can be made between the target product and non-specific DNA: Depending on the length and composition of the nucleotide, each DNA double-strand breaks into its two single strands at a temperature which is characteristic for it, the decomposition temperature. Since the double-stranded DNA product of specific PCR has a higher melting point than nonspecific primer dimers, a differentiation can be made based on the decrease in fluorescence with increasing temperature.

In contrast, detection is highly specific with fluorescence based probes, but also very expensive. With the TaqMan principle, the PCR approach utilizes, in addition to the PCR primers, a sequence-specific TaqMan hybridization probe, which is associated with a quencher and a reporter dye. The probe is complementary to a sequence which is located between the primers. In free solution, the fluorescence is suppressed by the proximity to the quencher. According to the FRET (fluorescence resonance energy transfer) principle the quencher absorbs the fluorescence emission of the excited fluorophore. However, if the probe hybridizes with the target sequence during PCR, it is hydrolyzed by the Taq polymerase, the reporter dye is spatially separated from the quencher, and upon excitation emits a detectable fluorescence. In the LightCycler principle the PCR mixture contains, in addition to the PCR primers, the two fluorescence labeled probes (donor and acceptor fluorescent dye). An externally measurable fluorescence signal arises only in the case of immediately adjacent hybridization of the two probes with the specific target sequence. In a subsequent decomposition curve analysis it is even possible to detect the existence and nature of single point mutations within the hybridization areas of the probes. Another example is the molecular beacons. These oligonucleotides contain, at the 5′ and 3′ end, sequences complementary to each other, which hybridize in the unbound state and form a hairpin structure. The reporter fluorophor and quencher, localized at both ends, are thus in immediate proximity. Only when the probe binds to the template are the two dyes spatially separated, so that after excitation fluorescence is again measurable. Scorpion and Sunrise primers are two other modifications for sequence-specific probes [Whitcombe et al., 1999]. 1999].

The quantitative determination of a template can be made by absolute or by relative quantification. In the case of absolute quantification measurement is made on the basis of external standards, such as plasmid DNA in different dilutions. The relative quantification, on other hand, uses so-called housekeeping genes or reference genes as reference [Huggett et al., 2005]. These reference genes are constantly expressed and thus provide an opportunity for standardization of different expression analysis. The selection of housekeeping genes must be made individually for each experiment. For the present invention Housekeeping genes are preferably the sequences listed in Table 2.

The generated experiment data are evaluated using device-specific software. For the graphic representation, the measured fluorescence intensity is plotted against the number of cycles. The resulting curve is divided into three areas. In the first phase, that is, at the beginning of the reaction, background noise still dominates, a signal of the PCR product is not yet detectable. The second phase corresponds to the exponential growth phase. In this segment, the DNA template is approximately doubled in every reaction step. Critical to the evaluation is the cycle at which detectable fluorescence first occurs and the exponential phase of amplification begins. This threshold cycle (CT) value, or Crossing Point, provides the basis for the calculation of the initial existing amount of target DNA. Therewith the software determines, in the case of an absolute quantification, the Crossing Points of various reference dilutions and quantifies on the basis of the calculated standard curve, the amount of template. In the last phase the reaction finally reaches a plateau.

Quantitative PCR is an important tool for gene expression studies in clinical research. With the ability to accurately quantify mRNA, it becomes possible in the search for new drugs to analyze the impact of certain factors on cells, differentiation of precursor cells into different cell types or monitor gene expression in host cells in response to infection. By comparing wild-type cells and cancer cells at the RNA level, genes can be identified in the cell culture which have a determinative influence cancer development. In routine laboratory diagnostics, real-time PCR is primarily used for the qualitative and quantitative detection of viruses and bacteria. In clinical routine, particularly in intensive care, the physician needs rapid and unequivocal findings. Using real-time PCR, tests can be performed that provide a result on the same day. This represents a huge advance in the clinical diagnosis of sepsis.

Besides the above described technical variants of the PCR method, there may also be used so-called isothermal amplification such as NASBA or SDA, or other technical options, can be used for the for the reproduction preceding the detection of the target sequence.

A preferred method for selecting the multi-gene biomarker sequences includes the following steps:
a) patient selection based on the extreme group method;
b) generation of at least one multi-gene biomarker;
c) determination of final multi-gene biomarkers.

A preferred method similar to the “in vitro diagnostic multivariate index assay” includes the following steps:
a) isolation of sample nucleic acids from a sample taken from a patient;
b) detection of gene activity by means of sequences of at least one condition and/or research issue specific multi-gene biomarker;
c) detection of gene activity for at least one internal reference gene to normalize gene activities measured to in b);
d) using an interpretation function for the gene activity normalized in c), in order to derive a condition and/or research issue specific index.

A preferred embodiment of the present invention is in a use in which the gene activity is determined using a hybridization method, and in particular using at least one microarray. The advantage of microarrays is in the higher information density of the biochip in comparison to the amplification process. Thus, for example, it is easily possible to provide several 100s of probes on a microarray in order to examine several issues at the same time in a single examination procedure.

The gene activity data obtained in accordance with the invention can also be used advantageously for electronic processing, for example, for recording in the electronic medical records.

A further embodiment of the invention is the use of recombinant or synthetic produced, specific nucleic acid sequences, partial sequences, individually or in smaller quantities, as multi-gene biomarkers in sepsis assays and/or evaluation of the effect and toxicity during drug screening and/or for manufacture of drugs and of substances and mixtures, which are provided as a therapeutic, for prevention and treatment of SIRS and sepsis.

For the inventive process (array technology and/or amplification process), the sample is selected from: tissue; body fluids, particularly blood, serum, plasma, urine, saliva or cells or cellular components; or a mixture thereof.

It is preferred that samples, particularly samples of cells, are subject to a lytic treatment to release their cell contents.

The person of ordinary skill in the art understands that individual features of the invention set forth in the claims are non-limiting and can be combined in any desired manner.

### Classification Methods

The theory of learning plays a key role in the field of statistics, data analysis and artificial intelligence with numerous applications in engineering. Classification methods are used mainly in two different tasks, the setting of boundaries of previously unknown classes (unsupervised learning, class discovery) and in the assignment specific data/samples/patients to a ready-defined class (class prediction) [Golub et al., 1999].

In class prediction patient data/samples/patients are used, that were assigned to previously existing or specified classes or groups (so-called training data set) to develop an analytical process (classification algorithm), which reflects the differences between the groups. Independent samples (so-called test set) are used to evaluate the performance quality of the classification rule. The process steps can be divided into the following:
1. an ideal data/sample/patient set is defined, in order to obtain the characteristic profiles of groups which are to be detected;
2. each group is then divided, so that two equal subsets, a training data set and a test data set, is created;
3. profiles for the training data set ideally contain data, which reflects the maximum difference between the groups;
4. the difference between the groups is quantified using an appropriate distance measurement and evaluated using an algorithm. This algorithm should lead to a classification rule, which assigns the data into the correct class with the highest specificity and sensitivity. Typical representatives of such algorithms in the field of supervised learning are Discriminant Analysis (DA), Random Forests (RF), Generalized Partial Least Squares (GPLS), Support Vector Machines (SVM) or k-Nearest Neighbor (kNN); and
5. finally, the quality of the classification rule is tested on a test set.

### DEFINITIONS

Discriminant analysis (DA): In the case of linear discriminant analysis we obtain a linear function, in the case of quadratic discriminant analysis (QDA) a quadratic discriminant function. The discriminant function is determined by the covariance matrix and the group means. In the case of the quadratic discriminant analysis it is additionally assumed, that even the covariance between the groups varies [Hastie et al., 2001].
Random Forests (RF): Classification using Random Forests is based on the combination of decision trees [Breiman, 2001]. The end of the algorithm is roughly as follows:
Selection by random drawing with replacement from a training data set (out-of-bag data).
At each node of the decision tree randomly select variables. Calculate based on these variables the best split or allocation of the training set to the classes.
After all the decision trees were generated, integrate the classification assignments of the individual decision trees into one classification assignment.
   Generalized Partial Least Squares (GPLS): The Generalized Partial Least Squares process [Ding and Gentleman, 2004] is a very flexible generalization of the multiple regression model. Due to the great flexibility, this method can be applied in many situations, even those in which the classical model fails.
   Support Vector Machine (SVM): The Support Vector Machine classifier is a generalized linear classifier. The input data is displayed in a higher dimensional space and in this space an optimal separating (hyper-) plane is constructed. These higher-dimensional space linear barriers are transformed into nonlinear barriers in the space on the basis of the input data, [Vapnik, 1999].
   k-nearest neighbor (k-Nearest Neighbors, kNN): With the method of k-nearest neighbors, the class membership of an observation (a patient) is decided on the basis of k-nearest neighbors located in its environment. The neighborhood is defined, as a rule, using the Euclidean distance, and the membership in the class can then be determined by a majority vote [Hastie et al., 2001].

The following a general concept is described, by which the inventive process is carried out. This person of ordinary skill knows that minor adjustments to the statistical methods may be necessary if other groups of patients and/or other issues are to be investigated. For the generation of the classification rule different statistical methods (discriminant analysis and/or Random Forests, etc.) as well as strategies are strategies used (single and multiple cross-validation, random Bootstrap samples, etc.)

Based on gene expression data, a method for determining a multi-gene biomarker should be developed, which mirrors an infectious complication such as, for example, sepsis. The biomarkers and the associated index value, also called the “score”, form the basis of a so-called “in vitro diagnostic multivariate index assays” [IVDMIA, FDA Guidelines, 2003] to improve the diagnosis of systemic infections. The classification rule resulting from the process should, in particular, make possible a differentiation of SIRS and sepsis patients—with improved sensitivity and specificity compared to the established biomarker procalcitonin—but is not limited to this issue.

To develop such a multi-gene biomarker, the following steps are necessary:

Step 1: Training data set. To detect the relationship between gene expression of certain examined genes and a disease, populations (cohorts) are defined, the presence or absence of which are most clearly representative of the disease. In the diagnosis of infectious complications usually sepsis patients (infectious) and patients with so-called sterile SIRS (non-infectious) are included in the study. Based on this definition, a plan is established for the collection or selection of the corresponding RNA samples. Of the selected samples, the gene expression profiles are measured on a suitable platform, pre-processed and subjected to quality control. Systematic measurement errors are corrected and outliers eliminated.
Step 2: Gene pre-selection. When generating a formal classifier based on microarray data a gene preselection is a key step, since only a small proportion of the measured genes contribute to the group distinction. Also, most classification methods have a gene selection as a precondition. By a precise gene selection, classification methods can be designed as simple as possible, and an overfitting against the training data (overfitting) can be avoided. For pre-selection of the genes to be classified, suitable filter options such as threshold of statistical inference, the minimum acceptable distance between the groups, the minimal signal intensity, inter alia, are determined. Only genes that meet these criteria will be considered for the classification.
Step 3: Classification procedures. Different classification methods are tested for their ability to classify or to differentiate with respect to the pathophysiological conditions to be distinguished. For this, cross-validation methods are used. A classification method with the smallest classification error is selected, wherein at the same time the smallest necessary number of genes is determined. As a reasonable rule it has been found that the number of genes should always be smaller than the number of samples in the training data set, to avoid overfitting. Finally, the resulting classification rule is defined.
Patient Selection The selection of patients is important in terms the establishment of the training data. In a preliminary study in the context of the present invention, initially a sensitivity about 75% in the training data set and about 65% in the test data set was reached. Diese This relatively poor classification quality was explained, however, as not being due to the weak optimization of the classifier, but due to a not precise enough selection of sepsis patients. According to this, sepsis patients were more often correctly classified after peritonitis than sepsis patients after a “VAP” (Ventilator-Associated Pneumonia). In fact, the infectious complication is present following peritonitis. In contrast, in the case of VAP, it is difficult to distinguish between a genuine infection and a colonization [Mayhall, 2001].

To assess the quality of patient selection, the principle of so-called extreme groups can be useful. In accordance therewith, in one study, only those patient groups are considered, which most clearly represent the studied effect. The chosen samples represent an idealized case, in which many of the effects occurring in practice (eg the frequency of the disease) are not taken into account. Liu [Liu et al., 2005] has proposed, as the training data set, to form extreme groups the basis of a microarray classifier. Using the survival analysis of cancer patients as an example, it has been shown that the use of extreme groups (patients who died after a short time vs. patients who have survived long) led to improved pre-selection of classification genes, and a higher classification quality, even though the training data set consisted of fewer profiles (patients), than in the usual case when all patients were taken into account (including also average survival times).

In the following it will be explained to what extent the selection of patients can influence the generating of a multi-gene biomarker for the diagnosis of infectious complications. In a study by the applicant patients who have developed sepsis after a major surgery were examined. Samples from the first day of sepsis diagnosis were compared with a sample from the first post-operative day. The significantly differentially expressed genes however reflect a mixed effect; the infectious complications are obscured by effects such as recovery from the surgical stress or the post-operative treatment. In the aforementioned pilot study, patients were enrolled in the training population with a clinical (not always microbiologically verified) sepsis diagnosis, leading to a mixing of the two studied groups (septic and controls) and lowering of the sensitivity. In the illustrative embodiment disclosed in U.S. Patent Application No. 20060246495, for the selection of the sepsis group, likewise the clinical diagnosis of sepsis was used. In addition, the severity of the disease between the group of sepsis patients and the control group of SIRS patients was not taken into considered. This may be the reason for the low classification quality and its dependence on its classification algorithm. In the study by Johnson [Johnson et al., 2007], patients divided into two groups after trauma, those with an infectious complication and those without an infection. The advantage of this study was that patients in the two groups differed little in pretreatment comorbidity. The pre-selection is not representative of all sepsis patients and the generalized applicability of this detected sepsis-relevant gene expression pattern to patients with a different background (to other risk groups) is not guaranteed. In general it must be assumed that in studies with different risk groups, various different classifiers must be generated. In the study by Tang [Tang et al., 2007a] the principle of extreme groups was used indirectly, in that only patients with a microbiologically verified a diagnosis of sepsis were included in the training data set. The sample-collection plan however lead to a small control group (one third of samples: 14 from 44). Accordingly, in the training set a specificity of 77% was achieved and in the independent test set (achieved under more realistic conditions) only 60%. The description of the groups of patients in the SIRS-Lab study and the study by Tang [Tang et al., 2007a] shows a further influence factor. It shows that, with regard to the focus of infection, heterogeneous sepsis groups are not balanced, but rather groups with different focus of infection are represented differently. In fact, in most cases in the intensive care unit (ICU), the lung (45-50%) or the abdomen (25%) is the focus of infection in sepsis diagnosis. Accordingly, these groups of patients are overrepresented in the studies; many other infection foci occur only sporadically. Similarly, in the control groups postoperative and trauma patients are especially represented, and other vulnerable groups are represented only by individual patients. The presented analysis shows that the groups of patients selected in all the studies do not clearly depict the infectious complications, which could explain the weakness in making the classification. On the other hand, it is clear from the summary that it is hardly possible, given the infectious complications, to take into consideration all factors in the selection of patient groups. For this reason, the following road to patient selection is proposed for the training data set.

### General Information on Material and Methods of the Present Invention:

### Patient Selection

The selection of representative samples was the core or nexus of the described process. Included (or excluded) in the training data set were patients with a microbiological verified diagnosis of infection (or non-infection) from two of the best-represented sepsis or control subgroups. Therewith the principle of extreme groups is applied not only for the main effect (infectious vs. non-infectious) but also for the control of the major influencing factors (stratification of populations). The advantage of this selection is, for the time being, that we herewith generated a classifier for the most common risk or disease groups. In addition, it is expected that a classifier, which reflects the systemic infection for a small in number but very different subgroup, can be applied to other patient groups. The selecting of the training data proceeded as follows. In the patient database of the applicant, in the time frame of two and a half years, 400 patients were treated in the ICU, in which a risk of sepsis was suspected, and the associated clinical data was documented in detail during the whole stay. The RNA samples were collected over 7-14 sepsis-relevant days. In approaching the PIRO concept [Levy et al., 2003], patients were retrospectively stratified according to the following criteria: (i) the indication that led to the admission to the ICU (postoperative complications, trauma or multiple trauma, suspicion of acute sepsis), (ii) if an infectious complication was diagnosed, what was the infectious focus, (iii) what was the response of the organism (the number of available SIRS criteria, shock treatment, PCT level, CRP level), (iv) how severe was the disease (SOFA, MODS-score). A search of the database revealed that, included in the study with an infectious complication (sepsis), especially were patients with pneumonia (40%) and following peritonitis (23%). Further focus appeared individually. These data correspond to the epidemiological studies of the German Sepsis Society, and therewith the collection was classified as representative. The patient data of these groups were independently tested by two doctors [to ACCP/SCCM, 1992, Levy et al., 2003; Calandra and Cohen, 2005] and the final patient selection was established. There were selected 29 patients with a microbiologically confirmed diagnosis and the first septic day was determined. The compilation of the severity criteria showed that for the patients' severe sepsis or a septic shock was diagnosed on the first day. They reached an average SOFA-value of 10, the sum of acute organ dysfunction was about 3. As control group, 29 risk patients were included after a bypass surgery. The first day with a severity similar to the sepsis groups was determined, but without signs of infection. An exemplary but not limiting compilation of important clinical and laboratory parameters for the selected patients is found in Table 1.

TABLE 1
Summary of clinical parameters of patients in the training data set.
The values correspond to the number, or, marked with a star,
the median (interquartile range), of values.

| | Sepsis | No sepsis |
|---|---|---|
| Number of patients | 29 | 29 |
| Mortality | 52% | 21% |
| Gender (m/f) | 22/7 | 20/9 |
| Age (y)* | 66 (13) | 68 (8) |
| SIRS-criteria* | 3 (0) | 3 (2) |
| SOFA-Score* | 10 (4) | 7 (4) |
| Number of organ | 3 (2) | 2 (2) |
| dysfunctions | | |
| PCT (ng/ml)* | 12 (24.32) | 1.82 (10.78) |
| CRP (mg/l)* | 194 (161) | 85.45 (88.675) |
| WBC (no/l)* | 12200 (11150) | 12800 (8700) |
| Apache II | 19 (6) | 13 (5) |
| Hypotension's treatment | 90% | 48% |
| | Sepsis- | Indication for ICU- |
| | focus: | admission |
| Peritonitis | 13 | Cardio-pulmonary bypass/ |
| Pneumonia | 8 | ICU-stay more than 3 days: |
| Mediastinitis | 4 | 22 |
| Myocarditis | 1 | Cardio-pulmonary bypass/ |
| Urosepsis | 1 | ICU-stay max. 3 days: 7 |
| Knee empyema | 1 | |

### Generation of the Classifier and Establishment of the SIQ Scores

On the way to development of classifier the following steps were undertaken:

Step 1: Quality Control: From the expert validated preselection of patients from the group of patients, the corresponding gene expression data was subjected to the various comparison analysis in order to exclude atypical hybridization results [Buness et al., 2005], whereby the final training data matrix was generated.
Step 2: Normalization or preprocessing of the data: For normalization, the average of the three selected housekeeper genes (R1, R2 and R3) was calculated for each sample. From this value the Ct value of each marker was derived. Each delta Ct value thus obtained reflects again the relative abundance of related target transcript with reference to the calibrator, wherein a positive delta Ct value means an abundance greater than the mean of references and a negative delta Ct value means an abundance less than the average of the references.
Step 3: Ranking: To rank the marker genes according to ability to discriminate, the linear discriminant analysis (LDA) [Hastie et al., 2001] was used together with the method of forward selection, whereby the ability to discriminate was evaluated using the F-value [Hocking, R R, 1976). This analysis step was repeated for 1000 bootstrap samples. The marker ranks determined in each repetition were averaged over the 1000 runs, and the marker candidates were arranged in ascending order according to the mean rank. This arrangement means that the marker with the smallest mean rank was the one which most frequently provided the greatest contribution to ability to distinguish and the marker with the highest mean rank contributed little for the differentiation in most repetitions.
Step 4: Classification: For the markers which yielded the best results in the ranking analysis, a discrimination function was determined based on the LDA. The corresponding weights are presented in Table 9.
Step 5: Internal Validation: In order of evaluate the quality of classification for the growing number of markers, a simple cross-validation was used.
Step 6: Establishment of the SIQ scores: Based on the discriminant function, a sepsis related diagnostic parameter, a so-called SIQ score (SIQ) was introduced as follows. For a new independent sample one is given, among other things, as a classification result, a dimension free value of the discriminant function. A positive value classifies the sample is as infectious and a negative value as non-infectious. For typical representatives of each group one obtains higher absolute values, for difficult to classify samples values reach close to zero. The scatter of the discriminant values correspond generally to the variability of the data matrix. Thus one arrives in the classification at discrimination values of about −5 to 5. In order to make the differences even more pronounced, the SIQ score (SIQ) is recorded as the 10-fold value of the discriminant function with the weights from the Table 9. Consequently, the values of the SIQ-test data vary from of about −50 up to 50.

The present invention will now be described in greater detail on the basis of examples and with reference to the sequence listing, which also forms a part of this description, without in any way limiting of this invention.

### Results

In the next step, the gene expression data from the patient database of the applicant, which were not used in the training data set, were subject to classification. This independent test data set consisted of 113 samples of 65 persons (see Tables 4 and 5). Samples from 38 sepsis patients were examined, which represented a broad spectrum of clinical phenotypes with risk of a generalized infection. In addition, samples covering the course of SIRS of 22 post-operative surgery patients as well as 5 healthy patients were analyzed.

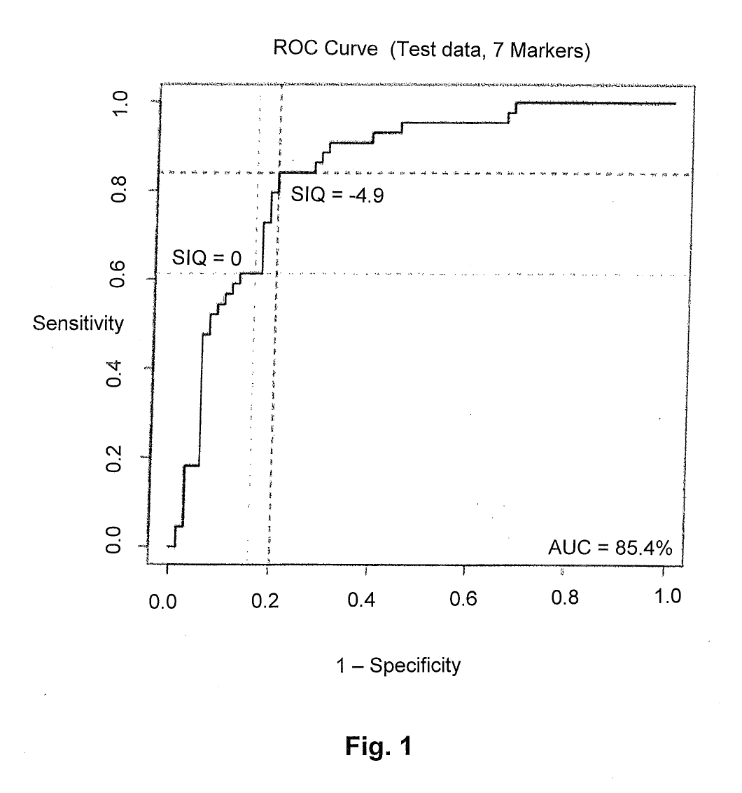

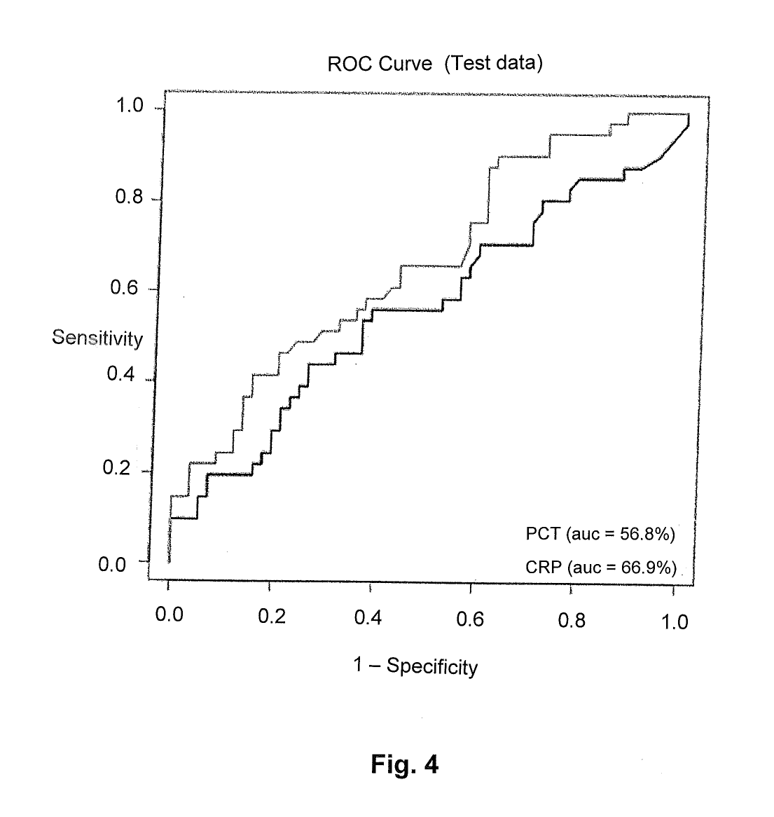

For this independent test data set, the best classification efficiency of 81.4% was achieved with the following seven markers: M6, M15, M9, M7, M2, M10, M4. The ROC curve for classification of test data is presented in FIG. 1. As a comparison, the ROC curve for classification of test data using PCT or CRP presented in FIG. 4. It can be seen from FIG. 4 that for both parameters, the area under the curve, which reflects the quality of the classification, is less than 70%, and thus is of little diagnostic relevancy.

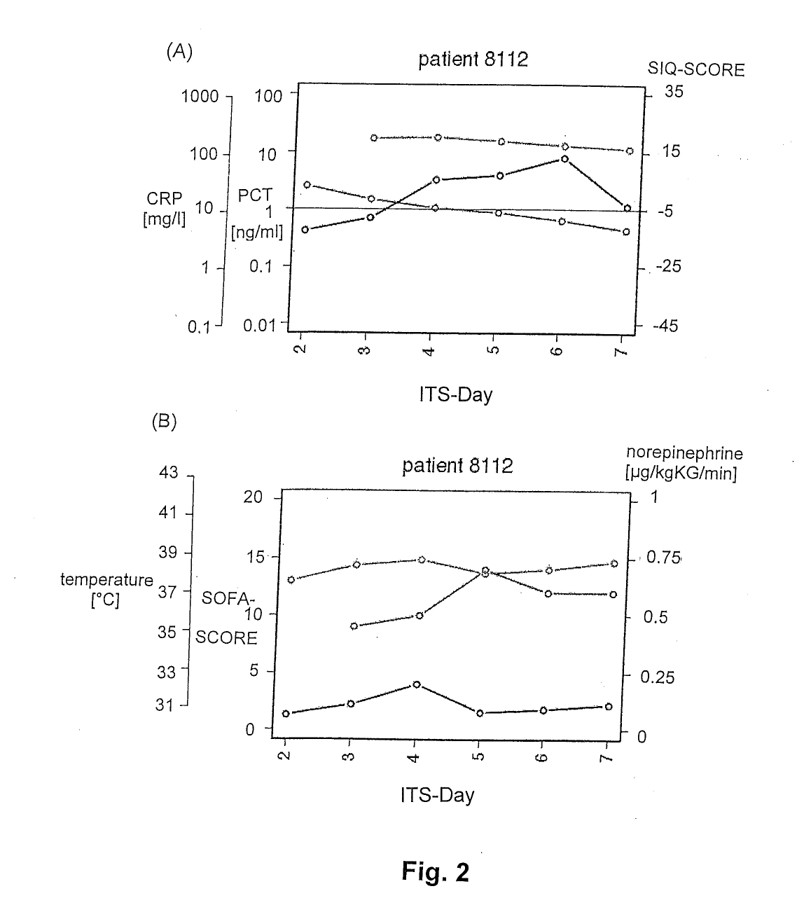

In FIG. 2 (patient 8112) the course of the SIQ-scores for one patient is presented, who has developed sepsis after surgery. From FIG. 2 it can be seen that the SIQ score exceeded the diagnostically-relevant threshold already two days before the clinical onset of sepsis. The course of other sepsis-related clinical parameters (PCT, CRP, SOFA, body temperature, shock treatment) are shown for comparison. From this comparison it can be seen that the SIQ score is the only parameter which reflects the early infectious complications. This demonstrates that the described invention can be used for the early detection of infectious complications such as sepsis and/or generalized infection.

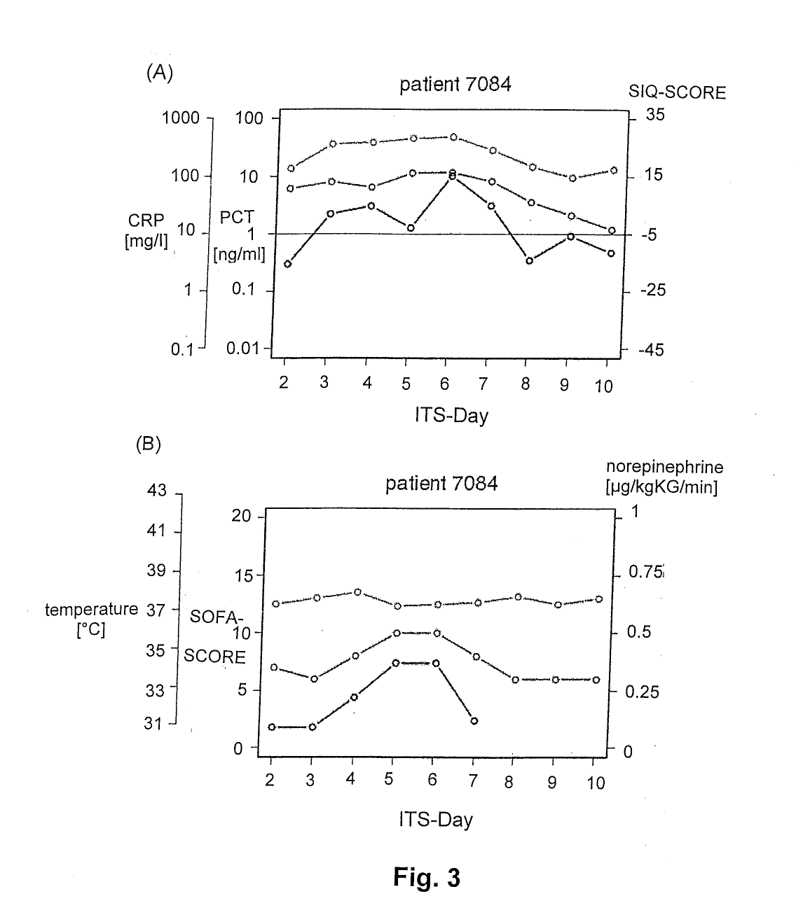

FIG. 3 (patient 7084) shows the course of the SIQ-scores for a patient, who developed postoperative sepsis, then a septic shock occurred, but following an acute phase recovered through a relevant treatment. From FIG. 3 it can be seen that the SIQ score exceeded the diagnostic threshold the day before the clinical onset of sepsis and in the acute phase remained above the threshold. After the acute phase of the SIQ-score fell below this threshold. This demonstrates that the described invention can be used for the monitoring and/or therapy control of, for example, antibiotic therapy and/or adjunctive clinical measures and/or operational sanitization or decontamination.

Other advantages and features of the present invention will become apparent from the description of illustrative embodiments and with reference to the drawing.

In the drawing there is show in:

FIG. 1 a ROC curve for classification of test data using SIQ scores;

FIG. 2 a representation of an exemplary course of an inventive Score

(SIQ-score) and the sepsis relevant clinical parameters PCT and CRP (FIG. 2A) as well as a SOFA-score, body temperature and catecholamine dosage (FIG. 2B) for a first patient;

FIG. 3 a representation of an exemplary course of an inventive Score (SIQ-score) and the sepsis relevant clinical parameters PCT and CRP (FIG. 3A) as well as a SOFA-score, body temperature and catecholamine dosage (FIG. 3B) for a second patient, and

FIG. 4 a ROC curve for classification of test data using PCT or CRP.

The present invention will now be using examples and with reference to the sequence listing, which is a part of this description is also explained in detail, without this implying any limitation of the invention.

FIG. 1 shows an ROC curve for classification of test data using the SIQ scores. In FIG. 1 the relationship between true positives (sensitivity) and false positives (1-specificity) is highlighted, dashed gray for the threshold of zero and dashed black for the best achieved classification of 81.4%.

FIG. 2 shows a course of SIQ scores of an exemplary patient as well as other sepsis-related clinical parameters PCT, CRP, SOFA, body temperature and the dosage of catecholamines (norepinephrine), which reflect shock-treatment. In Part A of the figure, the scale of each parameter adjusted so that the black horizontal center-line marks the diagnostically relevant threshold. Sepsis was diagnosed on day 6, the SIQ-score increased as early as day 4 over the threshold of −4.9.

FIG. 3 shows a course of the SIQ scores of another patient and relevant clinical parameters of sepsis namely PCT, CRP, SOFA, body temperature and the dosage of catecholamines (norepinephrine), which reflect the shock treatment. In Part A of the figure, the scale of each parameter is adjusted so that the black horizontal center-line marks the diagnostically relevant threshold. Sepsis was diagnosed on day 4 of ICU, the SIQ-score increased above the threshold of −4.9 already as early as day 3. After the acute phase, which ends with the discontinuation in catecholamines (shock treatment) at day 8, the SIQ-score falls below the threshold of −4.9.

FIG. 4 shows ROC curves for classification of test data using the parameters of the PCT or CRP. In FIG. 4 black represents the ROC curve for PCT and gray represents in the ROC curve for CRP. The area under the curve, the quality of the classification is reflected, which is 56.8% for PCT and 66.9% for CRP.

The following Table 2 shows the clear one-to-one association of the inventive marker polynucleotides to their transcript variants/cis regulatory sequences (isoforms), the genetic database access number and the SEQ ID NO. of the sequence listing.

TABLE 2

| Marker and | Transcript variants/cis- | | SEQ ID |
|---|---|---|---|
| Reference Genes | regulatory sequences | Accession Number | NO: |
| M2 | M2_1 | NM_001031700 | 1 |
| | M2_2 | NM_016613 | 2 |
| | M2_3 | NM_001128424 | 3 |
| M4 | M4_1 | NM_203330 | 4 |
| | M4_2 | NM_000611 | 5 |
| | M4_3 | NM_203329 | 6 |
| | M4_4 | NM_203331 | 7 |
| | M4_5 | NM_001127223 | 8 |
| | M4_6 | NM_001127225 | 9 |
| | M4_7 | NM_001127226 | 10 |
| | M4_8 | NM_001127227 | 11 |
| M6 | M6_1 | NM_001831 | 12 |
| | M6_2 | NM_203339 | 13 |
| M7 | M7_1 | NM_031311 | 14 |
| | M7_2 | NM_019029 | 15 |
| M9 | M9 | NM_006682 | 16 |
| M10 | M10 | NM_033554 | 17 |
| M15 | M15_1 | NM_003580 | 18 |
| | M15_2 | NM_001144772 | 19 |
| M3 | M3_A | NM_001123041 | 20 |
| | M3_B | NM_001123396 | 21 |
| M8 | M8 | NM_025209 | 22 |
| | M8_cis | AI807985 AI807985 | 23 |
| M12 | M12 | NM_002185 | 24 |
| | M12_cis | DB155561 | 25 |
| M13 | M13 | NM_001080394 | 26 |
| M16 | M16 | NM_003268 | 27 |
| M17 | M17 | NM_182491 | 28 |
| R1 | R1_A | Nm_001228 | 29 |
| | R1_B | NM_033355 | 30 |
| | R1_C | NM_033356 | 31 |
| | R1_E | NM_033358 | 32 |
| | R1_F | NM_001080124 | 33 |
| | R1_G | NM_001080125 | 34 |
| R2 | R2_1 | NM_002209 | 35 |
| | R2_2 | NM_001114380 | 36 |
| R3 | R3 | NM_003082 | 37 |

Table 3 shows, for each of the marker polynucleotides according to the invention, the primers (forward and reverse) for quantitative PCR and the resulting amplicon one and their one-to-one attribution to the respective SEQ ID of the sequence listing.

TABLE 3

| Marker and | Primers for quantitative | |
|---|---|---|
| Reference Gene | PCR/resulting amplicon | SEQ ID NO: |
| M2 | M2-fw | 38 |
| | M2-rev | 39 |
| | M2-Amplikon | 40 |
| M4 | M4-fw | 41 |
| | M4-rev | 42 |
| | M4-Amplikon | 43 |
| M6 | M6-fw | 44 |
| | M6-rev | 45 |
| | M6-Amplikon | 46 |
| M7 | M7-fw | 47 |
| | M7-rev | 48 |
| | M7-Amplikon | 49 |
| M9 | M9-fw | 50 |
| | M9-rev | 51 |
| | M9-Amplikon | 52 |
| M10 | M10-fw | 53 |
| | M10-rev | 54 |
| | M10-Amplikon | 55 |
| M15 | M15-fw | 56 |
| | M15-rev | 57 |
| | M15-Amplikon | 58 |
| M3 | M3-fw | 59 |
| | M3-rev | 60 |
| | M3-Amplikon | 61 |
| M8 | M8-fw | 62 |
| | M8-rev | 63 |
| | M8-Amplikon | 64 |
| M12 | M12-fw | 65 |
| | M12-rev | 66 |
| | M12-Amplikon | 67 |
| M13 | M13-fw | 68 |
| | M13-rev | 69 |
| | M13-Amplikon | 70 |
| M16 | M16-fw | 71 |
| | M16-rev | 72 |
| | M16-Amplikon | 73 |
| M17 | M17-fw | 74 |
| | M17-rev | 75 |
| | M17-Amplikon | 76 |
| R1 | R1-fw | 77 |
| | R1-rev | 78 |
| | R1-Amplikon | 79 |
| R2 | R2-fw | 80 |
| | R2-rev | 81 |
| | R2-Amplikon | 82 |
| R3 | R3-fw | 83 |
| | R3-rev | 84 |
| | R3-Amplikon | 85 |

### Biological Plausibility of the Identified Biomarkers

Functionally, the described biomarkers correlate with a high degree of significance with immunological and inflammatory signal pathways. A knowledge-based analysis of biomarker populations was carried out using the software Ingenuity Pathways Analysis (Ingenuity Systems, USAwww.ingenuity.com) in order to clarify the functional context of the identified markers. Based on the entire publicly available database-knowledge, the markers were categorized into functional networks and categories. The main categories of this marker population are the complement system, toll-like receptor signal induction, communication between cells of innate and adaptive immunity, TREM-1 signal transduction, and signal transduction via ceramide. The markers are thus, with high significance, involved with immunological and inflammatory processes, which underpins the relevance to the clinical picture of sepsis. Therewith an important prerequisite for biomarkers—the presence of biological plausibility—could be proved.

Theragnostics potential of biomarkers in the example of coagulation:

The analysis of the biological plausibility of the biomarkers showed that for M6 and M9 a functional role existed in the context of coagulation and fibrinolysis. Both processes are among the most deregulated physiologic functions in septic patients. A therapeutic option for patients with severe sepsis and organ failure involves treatment with activated protein C or thrombomodulin. M6 is overexpressed in septic patients, while at the same time subject to negative transcriptional control by activated protein C of the subject. M9 is suppressed in septic patients and may not be attributed to the meet the proscribed role of cleaving prothrombin for the provision of thrombin. Thrombin in turn, by association with thrombomodulin, is an important factor for the activation of protein C. Because of these close functional relationships, it would seem possible to look in clinical trials for patterns which would be characteristic for indicating receptiveness to use of the above treatment options. This theragnostics approach could make it possible to identify the responders and to save nonresponders from possible serious side effects. The identified markers having such an application thus also have a potential for decision-making about specific therapies of septic patients.

In the following the clinically relevant data for the studied group of patients are presented as Table 4:

TABLE 4

| | | | | | | | Survival | Length |
|---|---|---|---|---|---|---|---|---|
| | Age | | | | | Admission | Status | of Stay |
| Patient | (Years) | Gender | Apache | Postoperative indications | Non-surgical indications | Diagnosis | (ITS) | (Days) |
| 1013 | 59 | male | 22 | severe sepsis | | sepsis, unspecified | yes | 22 |
| 1015 | 71 | male | 29 | coronary blood vessel | | unstable angina | yes | 41 |
| | | | | intervention, thorax | | pectoris | | |
| 2042 | 81 | male | 15 | coronary blood vessel | | atherosclerotic heart | yes | 7 |
| | | | | intervention | | disease: one-blood | | |
| | | | | | | vessel disease | | |
| 5008 | 42 | male | 0 | | respiratory insufficiency | acute pancreatitis | no | 13 |
| | | | | | (infection), pancreatitis, | | | |
| | | | | | acute organ failure | | | |
| | | | | | (respiratory), acute organ | | | |
| | | | | | failure (metabolic), acute | | | |
| | | | | | organ failure (rel) | | | |
| 5009 | 57 | male | 21 | | Severe sepsis, respiratory | sepsis, unspecified | yes | 7 |
| | | | | | insufficiency (respiratory | | | |
| | | | | | arrest), respiratory | | | |
| | | | | | insufficiency (infection), | | | |
| | | | | | infectious liver failure, | | | |
| | | | | | acute organ failure | | | |
| | | | | | (respiratory), acute organ | | | |
| | | | | | failure (metabolic) | | | |
| 5010 | 67 | female | 0 | severe sepsis, postoperative- | | acute peritonitis | no | 31 |
| | | | | cardiovascular, postoperative | | | | |
| | | | | gastrointestinal, postoperative- | | | | |
| | | | | metabolic | | | | |
| 5018 | 71 | male | 28 | severe sepsis, coronary artery | | left heart failure | yes | 4 |
| | | | | intervention, postoperative- | | | | |
| | | | | cardiovascular, postoperative- | | | | |
| | | | | respiratory, postoperative-rel | | | | |
| 5019 | | female | | neurosurgical | | herniated disk | yes | |
| 5020 | | male | | neurosurgical | | herniated disk | yes | |
| 5023 | | female | | neurosurgical | | herniated disk | yes | |
| 6005 | 48 | female | 17 | severe sepsis | | acute | no | 28 |
| | | | | | | cholecystitis | | |
| 6008 | 62 | female | 14 | gastrointestinal | | peritonitis, | yes | 13 |
| | | | | | | unspecified | | |
| 6024 | 64 | male | 12 | | severe sepsis | sepsis, | yes | 6 |
| | | | | | | Escherichia coli | | |
| | | | | | | (E. Coli) | | |
| 6035 | 63 | male | 29 | severe sepsis, gastrointestinal | | perforation of the | no | 20 |
| | | | | | | intestine (non- | | |
| | | | | | | traumatic) | | |
| 6036 | 33 | male | 9 | polytrauma | | unspecified | yes | 20 |
| | | | | | | multiple injuries | | |
| 6056 | 76 | female | 23 | coronary artery intervention, cardiac | | aortic stenosis | yes | 36 |
| | | | | valve intervention | | | | |
| 6061 | 68 | female | 25 | coronary artery intervention, thorax | | aortic stenosis | no | 29 |
| 6063 | 59 | male | 17 | spine | | spinal cord | yes | 9 |
| | | | | | | compression, | | |
| | | | | | | unspecified | | |
| 6064 | 78 | male | 16 | | DHI-arrhythmia | other specified | yes | 36 |
| | | | | | | diseases or | | |
| | | | | | | pancreas | | |
| 6070 | 66 | male | 14 | severe sepsis, thorax | | chronic renal | yes | 19 |
| | | | | | | failure, | | |
| | | | | | | unspecified | | |
| 6075 | 73 | female | 22 | gastrointestinal | | Ileum, | yes | 47 |
| | | | | | | unspecified | | |
| 6104 | 40 | female | 17 | | severe sepsis, | acute respiratory | yes | 28 |
| | | | | | respiratory insufficiency | failure, not | | |
| | | | | | (asthma), respiratory | elsewhere | | |
| | | | | | insufficiency (aspiration), | specified | | |
| | | | | | respiratory insufficiency | | | |
| | | | | | (infection) | | | |
| 6120 | 54 | male | 18 | | severe sepsis | perforation of | no | 19 |
| | | | | | | the esophagus | | |
| 6124 | 69 | male | 15 | severe sepsis, thorax | | abnormal | yes | 13 |
| | | | | | | findings on | | |
| | | | | | | diagnostic | | |
| | | | | | | imaging of the | | |
| | | | | | | lung | | |
| 6126 | 39 | male | 23 | severe sepsis, | | adult respiratory | yes | 126 |
| | | | | polytrauma | | distress | | |
| | | | | | | syndrome | | |
| | | | | | | (ARDS) | | |
| 6141 | 70 | male | 21 | thorax | | emphysema, | no | 38 |
| | | | | | | unspecified | | |
| 7023 | 75 | male | 21 | coronary artery | | | no | 61 |
| | | | | intervention | | | | |
| 7040 | 70 | male | 21 | | | sepsis, | yes | 27 |
| | | | | | | unspecified | | |
| 7052 | 67 | female | 22 | | intracranial hemorrhage | subarachnoidal | yes | 33 |
| | | | | | | hemorrhage | | |
| | | | | | | from the anterior | | |
| | | | | | | communicating | | |
| | | | | | | artery | | |
| 7077 | 63 | male | 17 | | | malignant | yes | 38 |
| | | | | | | neoplasm floor | | |
| | | | | | | of mouth, | | |
| | | | | | | unspecified | | |
| 7079 | 77 | male | 26 | coronary artery | | | yes | 33 |
| | | | | intervention | | | | |
| 7084 | 69 | male | 17 | | | diseases of mitral and | yes | 24 |
| | | | | | | tricuspid valve, combined | | |
| 7096 | 85 | male | 18 | coronary artery | | atherosclerosis of the | yes | 8 |
| | | | | intervention | | arteries of extremities, | | |
| | | | | | | pelvis-leg type, with | | |
| | | | | | | gangrene | | |
| 7105 | 75 | female | 20 | severe sepsis | | sepsis, unspecified | yes | 10 |
| 7112 | 75 | female | 27 | coronary artery | | atherosclerotic heart | no | 67 |
| | | | | intervention | | disease: three-vessel | | |
| | | | | | | disease | | |
| 7119 | 64 | female | 14 | coronary artery | | unstable angina pectoris | yes | 6 |
| | | | | intervention | | | | |
| 7120 | 84 | female | 21 | severe sepsis | | malignant neoplasm on | no | 13 |
| | | | | | | rectdosigmoid, transition | | |
| 714 | 79 | female | 26 | gastrointestinal | | duodenal ulcer: chronic or | no | 7 |
| | | | | | | not described in more | | |
| | | | | | | detail, with perforation | | |
| 749 | 75 | female | 16 | cardiac valve | | aortic stenosis | yes | 27 |
| | | | | intervention, thorax | | | | |
| 8009 | 60 | male | 9 | coronary artery | | atherosclerotic heart | yes | 51 |
| | | | | intervention | | disease, without effective | | |
| | | | | | | hemodynamic stenosis | | |
| 8011 | 64 | male | 4 | coronary artery | | atherosclerotic heart | yes | 2 |
| | | | | intervention | | disease, without effective | | |
| | | | | | | hemodynamic stenosis | | |
| 8026 | 68 | female | 12 | coronary artery | | atherosclerotic heart | yes | 6 |
| | | | | intervention, cardiac valve | | disease, one-vessel | | |
| | | | | intervention | | disease | | |
| 8034 | 77 | female | 12 | coronary artery | | atherosclerotic heart | yes | 2 |
| | | | | intervention | | disease, without effective | | |
| | | | | | | hemodynamic stenosis | | |
| 8039 | 55 | female | 16 | cardiac valve intervention | | valvular aortic stenosis | yes | 7 |
| 8044 | 70 | male | 9 | coronary artery | | atherosclerotic heart | yes | 2 |
| | | | | intervention | | disease, without effective | | |
| | | | | | | hemodynamic stenosis | | |
| 8052 | 71 | male | 11 | coronary artery | | atherosclerotic heart | yes | 2 |
| | | | | intervention | | disease, without effective | | |
| | | | | | | hemodynamic stenosis | | |
| 8056 | 70 | female | 13 | cardiac valve intervention | | mitral insufficiency | yes | 5 |
| 8058 | 63 | female | 21 | cardiac valve intervention | | other aortic valve disease | yes | 5 |
| 8073 | 82 | male | 15 | coronary artery | | atherosclerotic heart | yes | 2 |
| | | | | intervention | | disease, without effective | | |
| | | | | | | hemodynamic stenosis | | |
| 8086 | 78 | male | 13 | coronary artery intervention | | atherosclerotic heart | yes | 6 |
| | | | | | | disease, one-vessel | | |
| | | | | | | disease | | |
| 8096 | 61 | male | 11 | coronary artery intervention, | | mitral stenosis | no | 12 |
| | | | | cardiac valve intervention | | | | |
| 8101 | 63 | male | 12 | coronary artery intervention | | atherosclerotic heart | no | 8 |
| | | | | | | disease, without effective | | |
| | | | | | | hemodynamic stenosis | | |
| 8102 | 70 | female | 17 | coronary artery intervention | | atherosclerotic heart | yes | 6 |
| | | | | | | disease, one-vessel | | |
| | | | | | | disease | | |
| 8103 | 54 | male | 6 | cardiac valve intervention | | aortic stenosis | yes | 2 |
| 8108 | 66 | male | 11 | coronary artery intervention | | atherosclerotic heart | yes | 2 |
| | | | | | | disease, without effective | | |
| | | | | | | hemodynamic stenosis | | |
| 8111 | 65 | male | 16 | coronary artery intervention | | atherosclerotic heart | yes | 14 |
| | | | | | | disease, without effective | | |
| | | | | | | hemodynamic stenosis | | |
| 8112 | 76 | male | 13 | coronary artery intervention | | atherosclerotic heart | no | 10 |
| | | | | | | disease, without effective | | |
| | | | | | | hemodynamic stenosis | | |
| 8116 | 80 | female | 18 | coronary artery intervention | | atherosclerotic heart | yes | 5 |
| | | | | | | disease, one-vessel | | |
| | | | | | | disease | | |
| 8122 | 67 | male | 17 | coronary artery intervention | | instable angina pectoris | yes | 5 |
| 920 | 74 | male | 23 | | severe sepsis | sepsis, unspecified | yes | 4 |

TABLE 5
a general description of the patients from the test data set were generally
recorded clinical parameters, the ITS treatment is justified.

| | | | | | | | | | Amount |
|---|---|---|---|---|---|---|---|---|---|
| | ICU- | PCT | CRP | SOFA- | Severity of | Amount. | Amount of | | SIRS- |
| Patient | Day | [ng/ml] | [mg/l] | SCORE | the Disease | ODF | Leucocytes | Group | Krit. |
| 1013 | 1 | 3.9 | | 8 | Severe | 1 | 23800 | S | 3 |
| | | | | | sepsis | | | | |
| 1015 | 10 | 5.12 | | 12 | Septic | 2 | 11100 | S | 3 |
| | | | | | shock | | | | |
| 2042 | 2 | 10.8 | 97.6 | 9 | SIRS | 2 | 18600 | C | 3 |
| 5008 | 6 | 10 | 200 | 11 | Severe | 2 | 17500 | S | 2 |
| | | | | | sepsis | | | | |
| 5009 | 3 | 2 | 250 | 8 | Severe | 1 | 7900 | S | 2 |
| | | | | | sepsis | | | | |
| 5010 | 2 | 0 | 164 | 8 | Septic | 3 | 11600 | S | 2 |
| | | | | | shock | | | | |
| 5018 | 2 | | 280 | | Septic | 3 | 17000 | S | 2 |
| | | | | | shock | | | | |
| 5019 | 1 | | | 0 | SIRS | | | C | |
| 5020 | 1 | | | | SIRS | | | C | |
| 5023 | 1 | | | | SIRS | | | C | |
| 6005 | 2 | | 290.1 | 8 | severe | 2 | 6900 | S | 3 |
| | | | | | Sepsis | | | | |
| 6008 | 2 | 6 | 111 | 7 | Septic | 3 | 16800 | S | 3 |
| | | | | | shock | | | | |
| 6024 | 2 | 2.46 | 49.8 | 10 | Severe | 3 | 10400 | S | 2 |
| | | | | | sepsis | | | | |
| 6035 | 3 | 4.72 | 103 | 13 | Septic | 3 | 16100 | S | 4 |
| | | | | | shock | | | | |
| 6036 | 10 | 0.65 | | 8 | Septic | 1 | 19200 | S | 2 |
| | | | | | shock | | | | |
| 6056 | 14 | 2.32 | 94.2 | 10 | Septic | 3 | 19300 | S | 4 |
| | | | | | shock | | | | |
| 6061 | 10 | 0.52 | 78.8 | 8 | Septic | 2 | 19900 | S | 4 |
| | | | | | shock | | | | |
| 6063 | 2 | 4.07 | 236 | 13 | Septic | 2 | 14200 | S | 4 |
| | | | | | shock | | | | |
| 6064 | 8 | 0.54 | 218 | 7 | Septic | 3 | 17400 | S | 4 |
| | | | | | shock | | | | |
| 6070 | 3 | 2.31 | 404 | 7 | Severe | 2 | 10600 | S | 1 |
| | | | | | Sepsis | | | | |
| 6075 | 3 | 37.6 | 269 | 9 | Septic | 3 | 37900 | S | 3 |
| | | | | | shock | | | | |
| 6104 | 7 | 32.9 | 325 | 6 | Sepsis | 1 | 11800 | S | 3 |
| 6120 | 3 | 36.8 | 478 | 12 | Septic | 3 | 11600 | S | 2 |
| | | | | | shock | | | | |
| 6124 | 5 | 0.3 | 159 | 9 | Septic | 3 | 9100 | S | 2 |
| | | | | | shock | | | | |
| 6126 | 10 | 86.2 | 80.4 | 10 | Septic | 4 | 13700 | S | 4 |
| | | | | | shock | | | | |
| 6141 | 5 | 1.18 | 247 | 7 | Septic | 3 | 13800 | S | 4 |
| | | | | | shock | | | | |
| 7023 | 9 | 0.3 | 124 | 10 | Septic | 2 | 14200 | S | 4 |
| | | | | | shock | | | | |
| 7040 | 2 | 13.5 | 304 | 11 | Septic | 2 | 28400 | S | 3 |
| | | | | | shock | | | | |
| 7052 | 12 | 0.45 | 229 | 9 | SIRS | 2 | 12400 | C | 2 |
| | 13 | 0.37 | 230 | 10 | SIRS | 2 | 14200 | C | 2 |
| | 14 | 0.47 | 234 | 8 | none | 2 | 11500 | C | 1 |
| 7077 | 9 | 0.65 | 335 | 10 | SIRS | 2 | 13700 | C | 3 |
| | 10 | 0.74 | 415 | 9 | Septic | 2 | 16400 | S | 3 |
| | | | | | shock | | | | |
| | 11 | 0.66 | 378 | 10 | Sepsis | 2 | 123000 | S | 4 |
| 7079 | 12 | 5.62 | 233 | 11 | Septic | 3 | 37000 | S | 3 |
| | | | | | shock | | | | |
| 7084 | 2 | 6.11 | 135 | 7 | Severe | 1 | 13100 | C | 3 |
| | | | | | SIRS | | | | |
| | 3 | 7.95 | 355 | 6 | SIRS | 1 | 14400 | C | 3 |
| | 4 | 6.41 | 379 | 8 | Septic | 1 | 10900 | S | 3 |
| | | | | | shock | | | | |
| | 5 | 11.4 | 449 | 10 | Septic | 2 | 10100 | S | 3 |
| | | | | | shock | | | | |
| 7096 | 2 | 1.24 | 134 | 7 | severe | 2 | 12400 | C | 2 |
| | | | | | SIRS | | | | |
| | 3 | 1.27 | 200 | 8 | severe | 1 | 12700 | C | 3 |
| | | | | | SIRS | | | | |
| | 4 | 0.62 | 164 | 6 | SIRS | 0 | 9600 | C | 2 |
| | 5 | 0.5 | 120 | 8 | Severe | 1 | 15700 | C | 3 |
| | | | | | SIRS | | | | |
| | 6 | 0.9 | 151 | 8 | severe | 1 | 14800 | C | 3 |
| | | | | | SIRS | | | | |
| | 7 | 0.96 | 177 | 7 | Sepsis | 0 | 15400 | S | 2 |
| | 8 | 1.1 | 215 | 7 | Sepsis | 0 | 20800 | S | 2 |
| 7105 | 1 | 3.29 | 311 | 12 | Septic | 3 | 14700 | S | 3 |
| | | | | | shock | | | | |
| 7112 | 23 | 0.9 | 56.7 | 9 | Severe | 2 | 13200 | S | 4 |
| | | | | | sepsis | | | | |
| 7119 | 3 | 1.31 | 288 | 7 | Severe | 2 | 19200 | C | 2 |
| | | | | | SIRS | | | | |
| | 4 | 0.61 | 295 | 5 | none | 1 | 11900 | C | 1 |
| | 5 | | 228 | 4 | SIRS | 0 | 9000 | C | 2 |
| 7120 | 2 | | 153 | 6 | Septic | 2 | 13000 | S | 2 |
| | | | | | shock | | | | |
| 714 | 1 | 0.89 | 111 | 9 | Septic | 3 | 17000 | S | 4 |
| | | | | | shock | | | | |
| 749 | 8 | 2.88 | 173 | 8 | Sepsis | 0 | 16000 | S | 3 |
| 8009 | 2 | 7.25 | 34.8 | 8 | SIRS | 2 | 10100 | C | 4 |
| | 3 | 5.38 | 206 | 6 | SIRS | 1 | 5800 | C | 4 |
| | 4 | 4.4 | 256 | 8 | SIRS | 2 | 16600 | C | 4 |
| | 5 | 7.67 | 288 | 10 | SIRS | 4 | 18900 | C | 4 |
| | 6 | 6.56 | 136 | 10 | SIRS | 2 | 15800 | C | 4 |
| | 7 | 3.97 | 162 | 9 | SIRS | 4 | 23700 | C | 4 |
| | 8 | 2.47 | 207 | 11 | SIRS | 4 | 26200 | C | 4 |
| | 11 | 9.84 | 207 | 11 | Septic | 4 | 28300 | S | 3 |
| | | | | | shock | | | | |
| 8011 | 2 | 0.3 | 82.9 | 5 | SIRS | 0 | 11400 | C | 2 |
| 8026 | 2 | 3.28 | 83.8 | 4 | SIRS | 2 | 8900 | C | 3 |
| | 3 | 3.01 | 205 | 7 | SIRS | 1 | 9200 | C | 3 |
| | 4 | 1.55 | 70 | 5 | SIRS | 0 | 10800 | C | 3 |
| | 5 | 0.77 | 39.6 | 4 | SIRS | 2 | 6900 | C | 3 |
| | 6 | 0.35 | 23.6 | 3 | SIRS | 0 | 7200 | C | 2 |
| 8034 | 2 | | 42.5 | 1 | SIRS | 0 | 9000 | C | 2 |
| 8039 | 2 | 1.39 | 53.8 | 6 | SIRS | 3 | 22500 | C | 4 |
| | 3 | 0.84 | 178 | 7 | SIRS | 2 | 19900 | C | 4 |
| | 4 | 0.86 | 197 | 8 | SIRS | 2 | 20800 | C | 4 |
| | 5 | 0.63 | 131 | 10 | SIRS | 2 | 17.4 | C | 3 |
| | 6 | 0.47 | 78.4 | 9 | SIRS | 2 | 14400 | C | 2 |
| 8044 | 2 | 0.43 | 48.3 | 1 | SIRS | 0 | 10700 | C | 3 |
| 8052 | 2 | | 84.7 | 0 | SIRS | 0 | 8700 | C | 2 |
| 8056 | 3 | 0.82 | 128 | 5 | SIRS | 1 | 22000 | C | 2 |
| 8058 | 3 | 22.7 | 72.7 | 11 | SIRS | 5 | 6300 | C | 2 |
| 8073 | 2 | 0.5 | 67.5 | 4 | SIRS | 1 | 6800 | C | 2 |
| 8086 | 2 | 0.35 | 37.7 | 5 | SIRS | 3 | 12400 | C | 3 |
| 8096 | 2 | 21.9 | 117 | 10 | SIRS | 3 | 15300 | C | 3 |
| | 3 | 14.5 | 294 | 12 | SIRS | 5 | 13500 | C | 4 |
| | 4 | 9.38 | 291 | 14 | SIRS | 5 | 13900 | C | 3 |
| 8101 | 3 | 1.27 | 92.9 | 8 | SIRS | 3 | 17900 | C | 4 |
| | 4 | 1.23 | 213 | 11 | SIRS | 5 | 15000 | C | 4 |
| | 5 | 1.42 | 195 | 11 | SIRS | 3 | 22600 | C | 4 |
| | 6 | 3.64 | 233 | 14 | sept. Shock | 5 | 39500 | S | 4 |
| | 7 | 6.59 | 184 | 17 | sept. Shock | 5 | 33600 | S | 4 |
| 8102 | 2 | 1.83 | 35.6 | 5 | SIRS | 3 | 13100 | C | 4 |
| 8103 | 2 | 0.52 | 55.8 | 1 | SIRS | 0 | 6900 | C | 2 |
| 8108 | 2 | 0.3 | 73.7 | 3 | SIRS | 0 | 7300 | C | 2 |
| 8111 | 2 | 6.82 | 67 | 7 | SIRS | 3 | 19700 | C | 4 |
| | 4 | 3.82 | 182 | | SIRS | 3 | 13800 | C | 2 |
| | 5 | 2.24 | 133 | 8 | SIRS | 3 | 14300 | C | 4 |
| | 6 | 0.82 | 67 | 5 | SIRS | 2 | 8700 | C | 3 |
| | 7 | 0.35 | 128 | 3 | Severe | 1 | 10400 | S | 2 |
| | | | | | Sepsis | | | | |
| | 8 | 0.3 | 98.2 | 5 | Severe | 3 | 19800 | S | 4 |
| | | | | | Sepsis | | | | |
| 8112 | 2 | 2.55 | | | SIRS | 3 | 14100 | C | 4 |
| | 3 | 1.49 | 168 | 9 | SIRS | 3 | 17400 | C | 4 |
| | 4 | 1.06 | 175 | 10 | SIRS | 4 | 13000 | C | 4 |
| | 5 | 0.88 | 151 | 14 | SIRS | 2 | 12500 | C | 4 |
| | 6 | 0.64 | 128 | 12 | Severe | 3 | 13300 | S | 4 |
| | | | | | Sepsis | | | | |
| | 7 | 0.44 | 113 | 12 | Septic | 3 | 9000 | S | 3 |
| | | | | | shock | | | | |
| 8116 | 2 | | 81.1 | 10 | SIRS | 5 | 15100 | C | 3 |
| 8122 | 4 | 0.3 | 171 | 4 | SIRS | 3 | 11200 | C | 2 |
| 920 | 2 | 1.26 | 124 | 10 | Septic | 5 | 10600 | S | 2 |
| | | | | | shock | | | | |

| | Noradrenalin- | | Likelihood- | |
|---|---|---|---|---|
| Patient | dose | CDCS | CDCS | Antibiotics |
| 1013 | 0.04 | superficial surgical | definitely, | Gentamicin, |
| | | wound infection, | definitely | Flucloxacillin, |
| | | spinal abscess | | Clindamycin, |
| | | | | Ceftriaxone |
| 1015 | 1.3 | pneumonia | definitely | Meropenem, Linezolid |
| 2042 | 0.26 | | | |
| 5008 | 0.2 | pneumonia | likely | Oxacillin, Tiem, Klion, |
| | | | | Diflucan |
| 5009 | 0.05 | pneumonia, | likely, | Ampicillin, |
| | | gastroenteritis | likely | Ciprofloxacin, |
| | | | | unknown: fortum, |
| | | | | Colimycin, unknown: |
| | | | | V-fend, Herpesin |
| 5010 | 0.06 | tracheobronchitis, | unlikely, | Cefuroxime, |
| | | GI tract infection, | definitely, | Metronidazole, Tiem, |
| | | intra abdominal | definitely | Vancomycin, |
| | | infection | | Sulperazon, Amikacin, |
| | | | | Flucozol |
| 5018 | 0.3 | endocarditis | likely | Amoxicillin, |
| | | | | Gentamicin |
| 5019 | 0.3 | | | |
| 5020 | 0.3 | | | |
| 5023 | 0.3 | | | |
| 6005 | 0.3 | cholecystitis | definitely | |
| 6008 | 0.56 | intra-abdominal | definitely | Cefuroxime, |
| | | infection | | Metronidazole |
| 6024 | 0.03 | meningitis/ | definitely, | Meropenem, |
| | | ventriculitis, | likely | Ceftriaxone |
| | | catheter sepsis | | |
| 6035 | 0.11 | intra-abdominal | definitely | Cefuroxime, |
| | | infection | | Metronidazole |
| 6036 | 0.16 | pneumonia | likely | Ciprofloxacin |
| 6056 | 0.31 | pneumonia | likely | Ciprofloxacin, |
| | | | | Imipenem |
| 6061 | 0.21 | pneumonia, | likely, | Flucozol, Imipenem |
| | | catheter | likely | |
| 6063 | 0.17 | deep surgical | likely, | Ceftriaxone, |
| | | wound infection, | likely, | Clindamycin |
| | | pneumonia, | likely | |
| | | tracheobronchitis | | |
| 6064 | 0.28 | deep surgical | likely, | Levofloxacin |
| | | wound infection, | likely, | |
| | | intra-abdominal | likely | |
| | | infection, | | |
| | | pancreatitis | | |
| 6070 | 0.093 | pneumonia, | likely, | Piperacillin/ |
| | | tracheobronchitis | likely, | Tazobactam, |
| | | | | Vancomycin |
| 6075 | 0.43 | intra-abdominal | likely, | Piperacillin/ |
| | | infection | | Tazobactam |
| 6104 | | pneumonia | likely | Imipenem, |
| | | | | Vancomycin |
| 6120 | 0.22 | intra-abdominal | likely | Cefuroxime |
| | | infection | | |
| 6124 | 0.22 | pneumonia | definite | |
| 6126 | 0.22 | superficial surgical | definite | |
| | | wound infection | | |
| 6141 | 0.17 | pneumonia | definitely | Piperacillin/ |
| | | | | Tazobactam |
| 7023 | 0.13 | pneumonia, | likely, | Piperacillin/ |
| | | bacteremia | likely | Tazobactam |
| 7040 | 0.36 | deep surgical | definitely, | Meropenem, |
| | | wound infection, | likely, | Vancomycin |
| | | pneumonia, | likely | |
| | | hematogenous | | |
| 7052 | 0.082 | | | Levofloxacin |
| | 0.1 | | | Levofloxacin |
| | 0.082 | | | Levofloxacin |
| 7077 | 0.27 | | | Amoxicillin/ |
| | | | | Clavulanic acid, |
| | | | | Gentamicin |
| | 0.25 | soft tissue | definitely | Amoxicillin/ |
| | | infection | | Clavulanic acid, |
| | | | | Gentamicin, Imipenem |
| | 0.2 | soft tissue | definitely | Gentamicin, Imipenem |
| | | infection | | |
| 7079 | 0.92 | mediastinitis | definitely | Levofloxacin, |
| | | | | Vancomycin |
| 7084 | 0.09 | | | Cefazolin |
| | 0.09 | | | Cefazolin |
| | 0.22 | pneumonia | likely | Cefazolin, Piperacillin/ |
| | | | | Tazobactam |
| | 0.37 | pneumonia | likely | Piperacillin/ |
| | | | | Tazobactam |
| 7096 | 0.17 | | | |
| | 0.062 | | | |
| | | | | Piperacillin/ |
| | | | | Tazobactam |
| | | pneumonia | likely | Piperacillin/ |
| | | | | Tazobactam |
| | | pneumonia | likely | Piperacillin/ |
| | | | | Tazobactam |
| 7105 | 0.25 | myocarditis/ | likely | Imipenem |
| | | pericarditis | | |
| 7112 | | pneumonia | likely | Cefepime, Flucozol, |
| | | | | Levofloxacin |
| 7119 | 0.16 | | | Piperacillin/ |
| | | | | Tazobactam |
| | 0.01 | | | |
| 7120 | 0.73 | intra-abdominal | likely | Cefuroxim, |
| | | infection | | Metronidazol |
| 714 | 0.87 | intra-abdominal | definitely | Metronidazole, |
| | | infection | | Cefuroxime |
| 749 | | tracheobronchitis | likely | Piperacillin/ |
| | | | | Tazobactam |
| 8009 | 0.17 | | | |
| | 0.22 | | | Piperacillin/ |
| | | | | Tazobactam |
| | 0.23 | | | Piperacillin/ |
| | | | | Tazobactam |
| | 0.16 | | | Meropenem, |
| | | | | Piperacillin/ |
| | | | | Tazobactam |
| | 0.2 | | | Meropenem |
| | 0.11 | | | Meropenem |
| | 0.39 | | | Meropenem |
| | 0.02 | pneumonia, | definite, | Meropenem |
| | | intra-abdominal | definite | |
| | | infection | | |
| 8011 | | | | |
| 8026 | | | | Cefazolin |
| | 0.052 | | | Cefazolin |
| | | | | Cefazolin |
| | | | | Cefazolin |
| | | | | Cefazolin |
| 8034 | | | | |
| 8039 | 0.12 | | | Cefazolin |
| | | | | Cefazolin |
| | | | | Cefazolin, Piperacillin/ |
| | | | | Tazobactam |
| | | | | Piperacillin/ |
| | | | | Tazobactam, |
| | | | | Erythromycin |
| | | | | Piperacillin/ |
| | | | | Tazobactam |
| 8044 | | | | Cefazolin |
| 8052 | | | | |
| 8056 | | | | Cefazolin |
| 8058 | | | | Cefazolin, Piperacillin/ |
| | | | | Tazobactam |
| 8073 | | | | |
| 8086 | 0.042 | | | |
| 8096 | 0.32 | | | Cefazolin |
| | 1.3 | | | Cefazolin |
| | 0.78 | | | Cefazolin |
| 8101 | 0.17 | | | Cefazolin, Piperacillin/ |
| | | | | Tazobactam |
| | 0.23 | | | Piperacillin/ |
| | | | | Tazobactam |
| | 0.46 | | | Piperacillin/ |
| | | | | Tazobactam |
| | 0.94 | | | Imipenem, |
| | | | | Vancomycin, |
| | | | | Piperacillin/ |
| | | | | Tazobactam |
| | 1 | | | Imipenem, |
| | | | | Vancomycin |
| 8102 | 0.016 | | | |
| 8103 | | | | Cefazolin |
| 8108 | | | | |
| 8111 | | | | Cefazolin |
| | 0.4 | pneumonia | likely | Ciprofloxacin |
| | 0.12 | | | Ciprofloxacin |
| | | | | Ciprofloxacin |
| | | tracheobronchitis | likely | Ciprofloxacin |
| | 0.089 | tracheobronchitis | likely | Ciprofloxacin, |
| | | | | Piperacillin/ |
| | | | | Tazobactam |
| 8112 | 0.066 | | | Cefazolin |
| | 0.11 | | | Cefazolin, Piperacillin/ |
| | | | | Tazobactam |
| | 0.2 | | | Piperacillin/ |
| | | | | Tazobactam |
| | 0.077 | | | Piperacillin/ |
| | | | | Tazobactam |
| | 0.09 | pneumonia | likely | Piperacillin/ |
| | | | | Tazobactam |
| | 0.11 | pneumonia | likely | |
| 8116 | | | | |
| 8122 | | | | |
| 920 | 0.031 | deep surgical | definitely | Meropenem |
| | | wound infection | | |

Table 5 shows sepsis-related clinical parameters from the progress curves of the patients taken from the test data set, clinical parameters are indicated by which the course of the disease is documented.

### EMBODIMENTS

### Example 1

### Establishment of a Classifier for the Identification of SIRS and Sepsis Patients with High Sensitivity/Specificity

### Patient Groups

In the first step of the analysis (training) samples from patients in an intensive care unit (ICU) were included. For the sepsis group patients with a microbiologically confirmed infection focus were selected, wherein the sample from the first day of sepsis was taken into consideration. As the control group patients were selected which, following a serious heart surgery (cardiopulmonary bypass, CPB), postoperatively developed a sterile SIRS. The control group was adjusted to the sepsis group regarding number of patients, age, gender distribution and severity of illness, so that the essential difference between the groups was the presence of an infectious complication. Each patient in the control group was represented by a single sample. The training data set consisted of 29 sepsis and 29 control cases. The main clinical parameters are summarized in Table 6.

In the second step (validation) a test data set was studied, which consisted of 113 samples from 65 persons (see Tables 4 and 5). Therein samples from further sepsis patients were examined, representing a broad spectrum of clinical phenotypes with risk of a generalized infection.

In addition, samples were selected with disease progression from sepsis SIRS. In the analysis, samples from at most the first two days of sepsis diagnosis were included.

As a control, there were used samples from the SIRS patients from the training group, technical repeats, samples of other postoperative cases and five healthy controls. With this selection of controls, the applicability of the method in a broad ranging phenotype is verified.

### Measurement of Gene Expression

Total RNA was isolated from the blood of patients and transcribed into cDNA. This was used as template in the assay. The marker candidates for the classification were summarized in Tables 2 and 3. At the end of the Table three so-called reference genes (also housekeeping genes) were added 3 (R1, R2 and R3). They allow a relative quantification of gene expression, which is an indicator of the abundance of target transcript in relation to a calibrator. Such reference genes are specific for every organism and every tissue are must be carefully selected for the desired application, which here is the differentiation between infectious and a non-infectious causes of systemic inflammatory response using human whole blood samples. Based on the gene expression profiles from whole blood of sepsis patients and control patients, the most stable genes with the lowest variability were selected and subject to normalization using quantitative PCR.

### Experimental Design

### Blood Collection and RNA Isolation:

The whole blood of patients was collected in PAXgene tubes (on PreAnalytiX, Hombrechtikon, CH) in an intensive care station for patients and stored in accordance with the manufacturer's instructions until processing. With PAXgene Blood RNA kits the RNA was isolated according to the manufacturer's specifications (Qiagen, Hilden, Germany), and stored at −80° C. until analysis.

### Reverse Transcription:

From each patient sample, 0.5 μg of total RNA was transcribed to complementary DNA (cDNA) with the reverse transcriptase Superscript II (Invitrogen Germany, Karlsruhe, Germany) in a 20 μl makeup (containing 1 μl 10 mM of dNTP-mix from Fermentas and 1 μl 0.5 μg/μl Oligo (dT)-Primer). The RNA was then removed by from the makeup by alkaline hydrolysis. The reaction mixtures were not purified, but filled with water to 50 μl.

### Real-Time PCR

The Platinum SYBR Green qPCR SuperMix-UDG—Kit of Invitrogen (Invitrogen Germany, Karlsruhe) was used. The patient's cDNA was diluted 1:25 with water, whereupon 1 μl of each which was used in the PCR. The samples were pipetted in three replicates.

| | |
|---|---|
| PCR-makeup per well (10 μl) | 2 μl template-cDNA 1:100 |
| | 1 μl forward primer, 10 nM |
| | 1 μl Fluorescein Reference Dye |
| | 2 μl Templat-cDNA 1:100 |
| | 5 μl Platinum SYBR Green qPCR |
| | SuperMix-UDG |

A master mix without template was produced, this was aliquoted into 9-μl aliquots on the PCR plate, to which were subsequently pipetted the patient cDNAs.

The subsequent PCR protocol consisted of the following steps:

| | | | |
|---|---|---|---|
| 95° C. | 2 min (activation of the polymerase) | | |
| 95° C. | 10 sec (denaturation) | | |
| 58° C. | 15 sec (annealing) | {close oversize brace} | 40 x |
| 72° C. | 20 sec (extension) | | |
| 55° C.-95° C. | 10 sec (creation of the melting curve | | |
| | increase of the initial temperature after | {close oversize brace} | 41 x |
| | each step by 1° C.) | | |

The iQ™5 Multicolor Real-Time PCR Detection System by BIORAD and associated evaluation software was used. The so-called Ct-values (number of cycles) were automatically calculated as measurement result by the program in the area of the linear increase of the plot curve. The measurements were stored in string format.

### Data Analysis:

The data analysis was carried out under the free Software R Project version R 2.8.0 (R.app GUI 1.26 (5256), S. Urbanek & S.M.Iacus, © R Foundation for Statistical Computing, 2008) available under www.r-project.org.

### Data Preprocessing:

The analysis of the input data matrices of the measured Ct values are presented in Tables 6 and 7 for respectively the training and test data sets. For normalizing, for each sample the average of the three selected housekeeper genes (R1, R2 and R3) was calculated. From this value, the Ct value of each individual marker is deducted. Each thus obtained delta Ct value thus reflects the relative abundance of the target transcript relative to the calibrator, wherein a positive delta Ct value indicates an abundance greater than the mean of the references and a negative delta Ct value indicates an abundance lower than the mean of the references.

TABLE 6
Ct values of the training data set per marker (mean of triple
determination) and group affiliation (last column).

| | M2 | M3 | M4 | M5 | M6 | M8 | M9 | M10 | M12 | M13 | M15 | M16 | M17 | R1 | R2 | R3 | Group |
|---|---|---|---|---|---|---|---|---|---|---|---|---|---|---|---|---|---|
| 2038_001 | 27.9 | 28.2 | 26.9 | 24.7 | 26.1 | 27.2 | 25.6 | 24.0 | 27.5 | 32.3 | 27.6 | 25.0 | 29.9 | 26.4 | 25.3 | 31.4 | no Sepsis |
| 8001_001 | 26.1 | 28.2 | 25.9 | 22.9 | 24.8 | 26.7 | 24.0 | 24.1 | 25.2 | 31.5 | 25.6 | 24.1 | 27.2 | 25.2 | 23.1 | 30.0 | no Sepsis |
| 8002_001 | 27.0 | 27.5 | 25.6 | 22.4 | 25.6 | 26.6 | 24.8 | 22.8 | 24.7 | 30.3 | 26.6 | 23.8 | 27.7 | 26.0 | 23.7 | 31.3 | no Sepsis |
| 8009_001 | 28.3 | 28.4 | 28.1 | 26.3 | 27.9 | 27.4 | 25.7 | 25.1 | 25.9 | 32.2 | 28.2 | 27.3 | 29.4 | 27.6 | 27.2 | 32.5 | no Sepsis |
| 8010_001 | 27.2 | 28.2 | 26.4 | 24.8 | 26.6 | 27.2 | 25.5 | 24.5 | 26.2 | 32.1 | 27.4 | 25.2 | 28.8 | 27.0 | 25.3 | 31.6 | no Sepsis |
| 8011_001 | 25.5 | 26.8 | 26.1 | 23.8 | 25.3 | 27.3 | 24.4 | 24.4 | 25.4 | 31.1 | 27.0 | 25.2 | 28.1 | 26.0 | 25.0 | 30.1 | no Sepsis |
| 8012_001 | 28.3 | 29.6 | 28.1 | 25.2 | 27.3 | 28.3 | 26.1 | 25.9 | 26.7 | 34.2 | 28.2 | 26.2 | 29.1 | 27.7 | 26.3 | 32.5 | no Sepsis |
| 8025_002 | 26.8 | 28.8 | 25.9 | 24.8 | 25.4 | 26.7 | 25.8 | 24.6 | 24.9 | 32.4 | 27.1 | 24.9 | 29.0 | 26.3 | 25.3 | 29.9 | no Sepsis |
| 8030_001 | 26.7 | 29.1 | 26.9 | 24.7 | 27.3 | 27.5 | 25.9 | 23.6 | 25.3 | 32.1 | 28.0 | 25.7 | 28.7 | 26.6 | 25.7 | 31.7 | no Sepsis |
| 8032_003 | 26.9 | 27.9 | 28.3 | 26.7 | 26.6 | 27.4 | 26.2 | 24.4 | 25.9 | 32.5 | 28.7 | 25.6 | 28.4 | 26.3 | 26.3 | 32.6 | no Sepsis |
| 8034_001 | 25.9 | 27.2 | 26.0 | 24.4 | 25.9 | 27.0 | 25.6 | 24.9 | 26.3 | 31.9 | 26.9 | 24.9 | 28.6 | 25.3 | 25.2 | 32.1 | no Sepsis |
| 8044_001 | 26.3 | 27.2 | 25.4 | 24.4 | 25.2 | 26.3 | 24.5 | 24.7 | 25.8 | 30.7 | 25.8 | 24.7 | 26.8 | 25.7 | 25.2 | 30.5 | no Sepsis |
| 8051_002 | 26.8 | 28.1 | 26.6 | 24.4 | 25.1 | 26.8 | 25.1 | 24.3 | 25.4 | 31.9 | 26.9 | 25.1 | 28.3 | 25.9 | 24.8 | 31.9 | no Sepsis |
| 8052_001 | 27.4 | 28.7 | 27.8 | 24.8 | 25.9 | 26.6 | 25.2 | 24.7 | 26.4 | 31.7 | 27.7 | 25.9 | 28.9 | 26.5 | 25.9 | 33.2 | no Sepsis |
| 8056_003 | 27.2 | 27.9 | 26.6 | 24.2 | 27.2 | 27.3 | 24.4 | 25.6 | 25.7 | 25.3 | 31.9 | 26.0 | 28.6 | 29.6 | 33.6 | 25.8 | no Sepsis |
| 8058_001 | 27.5 | 29.1 | 27.5 | 26.0 | 27.3 | 28.8 | 26.3 | 24.9 | 27.0 | 32.2 | 27.7 | 25.2 | 29.5 | 27.2 | 26.2 | 32.2 | no Sepsis |
| 8068_001 | 27.8 | 28.8 | 26.5 | 24.9 | 26.8 | 27.5 | 25.7 | 24.9 | 26.4 | 33.8 | 26.9 | 25.5 | 28.9 | 26.8 | 25.8 | 32.1 | no Sepsis |
| 8073_001 | 26.8 | 27.9 | 27.3 | 24.8 | 26.0 | 27.4 | 25.0 | 23.3 | 25.6 | 33.2 | 26.9 | 25.2 | 29.1 | 26.3 | 24.9 | 31.5 | no Sepsis |
| 8076_002 | 27.5 | 29.4 | 27.5 | 26.3 | 26.4 | 27.4 | 26.7 | 25.8 | 26.2 | 32.1 | 28.3 | 25.9 | 29.1 | 27.3 | 27.0 | 32.0 | no Sepsis |
| 8084_003 | 28.7 | 29.4 | 27.1 | 25.9 | 26.3 | 27.4 | 25.6 | 24.2 | 25.7 | 33.5 | 28.1 | 26.7 | 28.4 | 26.3 | 26.1 | 32.4 | no Sepsis |
| 8094_001 | 25.9 | 26.6 | 25.8 | 23.8 | 25.5 | 26.2 | 24.2 | 23.6 | 25.6 | 29.9 | 26.3 | 24.0 | 28.2 | 25.5 | 24.1 | 31.4 | no Sepsis |
| 8096_001 | 27.5 | 28.5 | 25.7 | 24.1 | 26.8 | 26.9 | 25.8 | 24.3 | 25.8 | 33.1 | 26.7 | 25.5 | 28.2 | 26.4 | 25.4 | 31.5 | no Sepsis |
| 8103_001 | 25.8 | 27.4 | 26.2 | 25.2 | 24.6 | 25.7 | 24.3 | 22.6 | 24.9 | 31.6 | 26.7 | 24.9 | 28.4 | 25.4 | 24.9 | 30.5 | no Sepsis |
| 8108_001 | 26.1 | 27.6 | 26.0 | 25.0 | 25.9 | 27.0 | 24.9 | 24.3 | 26.2 | 32.3 | 27.2 | 24.9 | 29.0 | 26.3 | 25.4 | 32.3 | no Sepsis |
| 8111_002 | 26.2 | 26.9 | 26.6 | 24.9 | 25.0 | 26.4 | 24.4 | 23.7 | 25.1 | 31.8 | 27.0 | 24.4 | 27.9 | 25.3 | 24.6 | 30.3 | no Sepsis |
| 8112_002 | 27.4 | 28.3 | 25.6 | 24.3 | 25.8 | 25.7 | 25.3 | 24.7 | 25.0 | 32.2 | 26.3 | 24.0 | 28.9 | 26.0 | 24.5 | 31.4 | no Sepsis |
| 8116_003 | 29.4 | 29.0 | 27.9 | 25.8 | 28.2 | 30.0 | 25.7 | 26.7 | 27.4 | 27.8 | 33.1 | 27.5 | 30.8 | 32.0 | 37.3 | 26.9 | no Sepsis |
| 8122_001 | 28.1 | 29.4 | 27.1 | 24.0 | 26.5 | 27.1 | 24.9 | 24.9 | 26.6 | 33.4 | 27.2 | 25.7 | 28.2 | 26.4 | 25.3 | 32.0 | no Sepsis |
| 814_001 | 26.5 | 27.3 | 26.0 | 25.8 | 25.9 | 24.5 | 24.4 | 23.4 | 23.1 | 31.0 | 25.8 | 25.7 | 27.5 | 25.2 | 24.5 | 30.0 | no Sepsis |
| 1014_002 | 27.8 | 29.1 | 25.6 | 24.5 | 28.4 | 26.2 | 26.8 | 25.4 | 24.8 | 32.0 | 27.7 | 25.0 | 29.0 | 26.3 | 25.9 | 31.4 | Sepsis |
| 1020_001 | 29.2 | 28.6 | 23.9 | 22.8 | 28.7 | 28.2 | 26.0 | 26.3 | 27.6 | 31.7 | 28.0 | 23.8 | 28.5 | 26.2 | 23.7 | 30.1 | Sepsis |
| 1021_001 | 27.0 | 26.3 | 26.3 | 23.6 | 28.6 | 25.9 | 24.8 | 26.1 | 25.8 | 31.0 | 30.8 | 26.0 | 28.8 | 27.8 | 31.2 | 23.7 | Sepsis |
| 6008_001 | 27.7 | 28.6 | 25.3 | 23.0 | 26.5 | 27.3 | 25.3 | 23.6 | 25.1 | 32.0 | 27.2 | 24.6 | 29.2 | 26.2 | 24.5 | 30.9 | Sepsis |
| 6009_001 | 28.9 | 29.8 | 25.0 | 23.6 | 28.6 | 27.3 | 26.6 | 27.3 | 25.2 | 31.9 | 27.4 | 24.1 | 28.9 | 25.5 | 24.6 | 30.7 | Sepsis |
| 6025_001 | 28.9 | 27.6 | 24.7 | 23.3 | 27.5 | 26.7 | 26.4 | 26.7 | 23.5 | 26.7 | 26.3 | 24.5 | 26.5 | 28.3 | 26.2 | 28.7 | Sepsis |
| 6032_001 | 28.5 | 30.4 | 27.0 | 23.7 | 27.9 | 29.0 | 26.6 | 25.2 | 25.3 | 34.6 | 28.0 | 25.3 | 29.0 | 27.4 | 25.2 | 32.1 | Sepsis |
| 6035_001 | 27.0 | 28.2 | 24.5 | 24.1 | 25.7 | 26.4 | 26.0 | 25.5 | 26.6 | 32.1 | 26.9 | 24.2 | 28.0 | 25.8 | 24.8 | 31.0 | Sepsis |
| 6040_001 | 27.4 | 28.1 | 23.6 | 21.7 | 28.4 | 26.1 | 27.1 | 23.8 | 24.4 | 29.6 | 26.0 | 25.3 | 26.5 | 25.5 | 25.3 | 30.1 | Sepsis |
| 6046_001 | 28.6 | 30.0 | 26.1 | 24.4 | 28.7 | 27.5 | 27.7 | 25.6 | 25.4 | 32.5 | 28.5 | 25.8 | 28.5 | 26.6 | 26.2 | 31.6 | Sepsis |
| 6048_001 | 29.6 | 30.7 | 27.0 | 26.7 | 29.7 | 29.4 | 28.2 | 26.9 | 26.9 | 34.5 | 28.9 | 26.3 | 29.8 | 26.8 | 27.4 | 32.7 | Sepsis |
| 6062_001 | 29.6 | 31.7 | 25.9 | 23.9 | 29.6 | 27.8 | 27.4 | 26.7 | 27.4 | 33.6 | 28.6 | 24.3 | 28.6 | 26.5 | 25.6 | 31.7 | Sepsis |
| 6065_001 | 28.1 | 28.6 | 26.2 | 24.4 | 28.9 | 30.0 | 26.5 | 26.2 | 26.5 | 34.8 | 27.7 | 24.9 | 29.4 | 26.4 | 25.7 | 32.5 | Sepsis |
| 6070_001 | 28.4 | 29.9 | 26.8 | 25.2 | 27.6 | 28.0 | 26.8 | 26.9 | 26.0 | 34.9 | 28.8 | 25.9 | 29.9 | 27.4 | 26.4 | 32.3 | Sepsis |
| 6073_001 | 29.7 | 31.6 | 26.3 | 24.9 | 29.7 | 29.3 | 28.9 | 26.9 | 25.7 | 33.7 | 29.9 | 25.9 | 29.9 | 27.4 | 27.6 | 32.4 | Sepsis |
| 6075_001 | 31.6 | 33.2 | 24.0 | 23.4 | 32.5 | 27.2 | 28.5 | 26.9 | 27.3 | 34.0 | 27.3 | 24.3 | 27.2 | 26.1 | 26.5 | 32.8 | Sepsis |
| 6078_001 | 27.3 | 30.0 | 24.7 | 24.6 | 28.0 | 27.8 | 26.4 | 25.2 | 26.4 | 31.6 | 28.0 | 24.8 | 28.0 | 26.7 | 26.2 | 32.6 | Sepsis |
| 6081_001 | 30.4 | 30.7 | 27.2 | 24.2 | 30.1 | 29.3 | 29.1 | 27.8 | 27.5 | 33.6 | 30.4 | 26.9 | 29.6 | 29.4 | 28.7 | 33.9 | Sepsis |
| 6082_001 | 30.5 | 32.5 | 27.8 | 26.1 | 29.6 | 30.4 | 28.0 | 28.0 | 28.5 | 33.0 | 29.8 | 27.8 | 30.6 | 28.1 | 26.4 | 31.5 | Sepsis |
| 6084_001 | 28.2 | 27.4 | 25.2 | 24.7 | 27.3 | 28.4 | 26.0 | 26.8 | 25.9 | 27.6 | 32.1 | 25.5 | 29.2 | 29.0 | 32.0 | 24.8 | Sepsis |
| 6085_001 | 29.1 | 30.0 | 27.2 | 24.4 | 28.1 | 28.5 | 27.1 | 26.0 | 26.0 | 33.5 | 29.1 | 25.2 | 29.3 | 27.5 | 26.1 | 32.0 | Sepsis |
| 6098_001 | 28.2 | 29.1 | 26.8 | 24.9 | 25.7 | 28.5 | 26.4 | 25.0 | 25.8 | 32.9 | 27.7 | 24.8 | 29.6 | 26.5 | 24.8 | 32.2 | Sepsis |
| 6104_001 | 28.4 | 27.7 | 26.7 | 23.9 | 26.8 | 27.9 | 26.3 | 24.1 | 25.8 | 25.2 | 31.3 | 25.7 | 28.4 | 29.8 | 32.4 | 25.0 | Sepsis |
| 6110_001 | 28.7 | 31.0 | 26.6 | 24.2 | 27.9 | 27.3 | 28.0 | 26.8 | 26.8 | 33.7 | 28.9 | 26.4 | 28.9 | 27.8 | 26.6 | 33.6 | Sepsis |
| 6115_001 | 30.1 | 31.2 | 28.8 | 24.9 | 27.9 | 29.3 | 26.7 | 26.1 | 27.5 | 34.1 | 29.2 | 27.3 | 31.1 | 28.4 | 26.6 | 32.4 | Sepsis |
| 6125_001 | 28.3 | 29.2 | 26.5 | 23.4 | 26.9 | 27.8 | 25.5 | 24.7 | 26.7 | 32.4 | 28.2 | 25.3 | 29.2 | 27.0 | 25.4 | 31.3 | Sepsis |
| 829_001 | 28.7 | 31.3 | 25.5 | 24.8 | 28.5 | 27.1 | 26.1 | 26.5 | 25.5 | 31.3 | 27.5 | 24.6 | 27.6 | 24.9 | 24.2 | 30.5 | Sepsis |
| 942_001 | 30.0 | 31.9 | 27.7 | 25.2 | 27.7 | 27.7 | 25.8 | 25.4 | 25.9 | 33.8 | 28.7 | 25.8 | 28.8 | 26.8 | 24.6 | 31.7 | Sepsis |
| 987_001 | 26.7 | 28.2 | 25.8 | 22.5 | 25.5 | 26.0 | 24.9 | 24.6 | 26.0 | 31.9 | 26.7 | 24.2 | 28.6 | 26.2 | 24.2 | 31.7 | Sepsis |
| 2038_001 | 27.9 | 28.2 | 26.9 | 24.7 | 26.1 | 27.2 | 25.6 | 24.0 | 27.5 | 32.3 | 27.6 | 25.0 | 29.9 | 26.4 | 25.3 | 31.4 | No sepsis |
| 8001_001 | 26.1 | 28.2 | 25.9 | 22.9 | 24.8 | 26.7 | 24.0 | 24.1 | 25.2 | 31.5 | 25.6 | 24.1 | 27.2 | 25.2 | 23.1 | 30.0 | No sepsis |
| 8002_001 | 27.0 | 27.5 | 25.6 | 22.4 | 25.6 | 26.6 | 24.8 | 22.8 | 24.7 | 30.3 | 26.6 | 23.8 | 27.7 | 26.0 | 23.7 | 31.3 | No sepsis |
| 8009_001 | 28.3 | 28.4 | 28.1 | 26.3 | 27.9 | 27.4 | 25.7 | 25.1 | 25.9 | 32.2 | 28.2 | 27.3 | 29.4 | 27.6 | 27.2 | 32.5 | No sepsis |
| 8010_001 | 27.2 | 28.2 | 26.4 | 24.8 | 26.6 | 27.2 | 25.5 | 24.5 | 26.2 | 32.1 | 27.4 | 25.2 | 28.8 | 27.0 | 25.3 | 31.6 | No sepsis |
| 8011_001 | 25.5 | 26.8 | 26.1 | 23.8 | 25.3 | 27.3 | 24.4 | 24.4 | 25.4 | 31.1 | 27.0 | 25.2 | 28.1 | 26.0 | 25.0 | 30.1 | No sepsis |
| 8012_001 | 28.3 | 29.6 | 28.1 | 25.2 | 27.3 | 28.3 | 26.1 | 25.9 | 26.7 | 34.2 | 28.2 | 26.2 | 29.1 | 27.7 | 26.3 | 32.5 | No sepsis |
| 8025_002 | 26.8 | 28.8 | 25.9 | 24.8 | 25.4 | 26.7 | 25.8 | 24.6 | 24.9 | 32.4 | 27.1 | 24.9 | 29.0 | 26.3 | 25.3 | 29.9 | No sepsis |
| 8030_001 | 26.7 | 29.1 | 26.9 | 24.7 | 27.3 | 27.5 | 25.9 | 23.6 | 25.3 | 32.1 | 28.0 | 25.7 | 28.7 | 26.6 | 25.7 | 31.7 | No sepsis |
| 8032_003 | 26.9 | 27.9 | 28.3 | 26.7 | 26.6 | 27.4 | 26.2 | 24.4 | 25.9 | 32.5 | 28.7 | 25.6 | 28.4 | 26.3 | 26.3 | 32.6 | No sepsis |
| 8034_001 | 25.9 | 27.2 | 26.0 | 24.4 | 25.9 | 27.0 | 25.6 | 24.9 | 26.3 | 31.9 | 26.9 | 24.9 | 28.6 | 25.3 | 25.2 | 32.1 | No sepsis |
| 8044_001 | 26.3 | 27.2 | 25.4 | 24.4 | 25.2 | 26.3 | 24.5 | 24.7 | 25.8 | 30.7 | 25.8 | 24.7 | 26.8 | 25.7 | 25.2 | 30.5 | No sepsis |
| 8051_002 | 26.8 | 28.1 | 26.6 | 24.4 | 25.1 | 26.8 | 25.1 | 24.3 | 25.4 | 31.9 | 26.9 | 25.1 | 28.3 | 25.9 | 24.8 | 31.9 | No sepsis |
| 8052_001 | 27.4 | 28.7 | 27.8 | 24.8 | 25.9 | 26.6 | 25.2 | 24.7 | 26.4 | 31.7 | 27.7 | 25.9 | 28.9 | 26.5 | 25.9 | 33.2 | No sepsis |
| 8056_003 | 27.2 | 27.9 | 26.6 | 24.2 | 27.2 | 27.3 | 24.4 | 25.6 | 25.7 | 25.3 | 31.9 | 26.0 | 28.6 | 29.6 | 33.6 | 25.8 | No sepsis |
| 8058_001 | 27.5 | 29.1 | 27.5 | 26.0 | 27.3 | 28.8 | 26.3 | 24.9 | 27.0 | 32.2 | 27.7 | 25.2 | 29.5 | 27.2 | 26.2 | 32.2 | No sepsis |
| 8068_001 | 27.8 | 28.8 | 26.5 | 24.9 | 26.8 | 27.5 | 25.7 | 24.9 | 26.4 | 33.8 | 26.9 | 25.5 | 28.9 | 26.8 | 25.8 | 32.1 | No sepsis |
| 8073_001 | 26.8 | 27.9 | 27.3 | 24.8 | 26.0 | 27.4 | 25.0 | 23.3 | 25.6 | 33.2 | 26.9 | 25.2 | 29.1 | 26.3 | 24.9 | 31.5 | No sepsis |
| 8076_002 | 27.5 | 29.4 | 27.5 | 26.3 | 26.4 | 27.4 | 26.7 | 25.8 | 26.2 | 32.1 | 28.3 | 25.9 | 29.1 | 27.3 | 27.0 | 32.0 | No sepsis |
| 8084_003 | 28.7 | 29.4 | 27.1 | 25.9 | 26.3 | 27.4 | 25.6 | 24.2 | 25.7 | 33.5 | 28.1 | 26.7 | 28.4 | 26.3 | 26.1 | 32.4 | No sepsis |
| 8094_001 | 25.9 | 26.6 | 25.8 | 23.8 | 25.5 | 26.2 | 24.2 | 23.6 | 25.6 | 29.9 | 26.3 | 24.0 | 28.2 | 25.5 | 24.1 | 31.4 | No sepsis |
| 8096_001 | 27.5 | 28.5 | 25.7 | 24.1 | 26.8 | 26.9 | 25.8 | 24.3 | 25.8 | 33.1 | 26.7 | 25.5 | 28.2 | 26.4 | 25.4 | 31.5 | No sepsis |
| 8103_001 | 25.8 | 27.4 | 26.2 | 25.2 | 24.6 | 25.7 | 24.3 | 22.6 | 24.9 | 31.6 | 26.7 | 24.9 | 28.4 | 25.4 | 24.9 | 30.5 | No sepsis |
| 8108_001 | 26.1 | 27.6 | 26.0 | 25.0 | 25.9 | 27.0 | 24.9 | 24.3 | 26.2 | 32.3 | 27.2 | 24.9 | 29.0 | 26.3 | 25.4 | 32.3 | No sepsis |
| 8111_002 | 26.2 | 26.9 | 26.6 | 24.9 | 25.0 | 26.4 | 24.4 | 23.7 | 25.1 | 31.8 | 27.0 | 24.4 | 27.9 | 25.3 | 24.6 | 30.3 | No sepsis |
| 8112_002 | 27.4 | 28.3 | 25.6 | 24.3 | 25.8 | 25.7 | 25.3 | 24.7 | 25.0 | 32.2 | 26.3 | 24.0 | 28.9 | 26.0 | 24.5 | 31.4 | No sepsis |
| 8116_003 | 29.4 | 29.0 | 27.9 | 25.8 | 28.2 | 30.0 | 25.7 | 26.7 | 27.4 | 27.8 | 33.1 | 27.5 | 30.8 | 32.0 | 37.3 | 26.9 | No sepsis |
| 8122_001 | 28.1 | 29.4 | 27.1 | 24.0 | 26.5 | 27.1 | 24.9 | 24.9 | 26.6 | 33.4 | 27.2 | 25.7 | 28.2 | 26.4 | 25.3 | 32.0 | No sepsis |
| 814_001 | 26.5 | 27.3 | 26.0 | 25.8 | 25.9 | 24.5 | 24.4 | 23.4 | 23.1 | 31.0 | 25.8 | 25.7 | 27.5 | 25.2 | 24.5 | 30.0 | No sepsis |
| 1014_002 | 27.8 | 29.1 | 25.6 | 24.5 | 28.4 | 26.2 | 26.8 | 25.4 | 24.8 | 32.0 | 27.7 | 25.0 | 29.0 | 26.3 | 25.9 | 31.4 | Sepsis |
| 1020_001 | 29.2 | 28.6 | 23.9 | 22.8 | 28.7 | 28.2 | 26.0 | 26.3 | 27.6 | 31.7 | 28.0 | 23.8 | 28.5 | 26.2 | 23.7 | 30.1 | Sepsis |
| 1021_001 | 27.0 | 26.3 | 26.3 | 23.6 | 28.6 | 25.9 | 24.8 | 26.1 | 25.8 | 31.0 | 30.8 | 26.0 | 28.8 | 27.8 | 31.2 | 23.7 | Sepsis |
| 6008_001 | 27.7 | 28.6 | 25.3 | 23.0 | 26.5 | 27.3 | 25.3 | 23.6 | 25.1 | 32.0 | 27.2 | 24.6 | 29.2 | 26.2 | 24.5 | 30.9 | Sepsis |
| 6009_001 | 28.9 | 29.8 | 25.0 | 23.6 | 28.6 | 27.3 | 26.6 | 27.3 | 25.2 | 31.9 | 27.4 | 24.1 | 28.9 | 25.5 | 24.6 | 30.7 | Sepsis |
| 6025_001 | 28.9 | 27.6 | 24.7 | 23.3 | 27.5 | 26.7 | 26.4 | 26.7 | 23.5 | 26.7 | 26.3 | 24.5 | 26.5 | 28.3 | 26.2 | 28.7 | Sepsis |
| 6032_001 | 28.5 | 30.4 | 27.0 | 23.7 | 27.9 | 29.0 | 26.6 | 25.2 | 25.3 | 34.6 | 28.0 | 25.3 | 29.0 | 27.4 | 25.2 | 32.1 | Sepsis |
| 6035_001 | 27.0 | 28.2 | 24.5 | 24.1 | 25.7 | 26.4 | 26.0 | 25.5 | 26.6 | 32.1 | 26.9 | 24.2 | 28.0 | 25.8 | 24.8 | 31.0 | Sepsis |
| 6040_001 | 27.4 | 28.1 | 23.6 | 21.7 | 28.4 | 26.1 | 27.1 | 23.8 | 24.4 | 29.6 | 26.0 | 25.3 | 26.5 | 25.5 | 25.3 | 30.1 | Sepsis |
| 6046_001 | 28.6 | 30.0 | 26.1 | 24.4 | 28.7 | 27.5 | 27.7 | 25.6 | 25.4 | 32.5 | 28.5 | 25.8 | 28.5 | 26.6 | 26.2 | 31.6 | Sepsis |
| 6048_001 | 29.6 | 30.7 | 27.0 | 26.7 | 29.7 | 29.4 | 28.2 | 26.9 | 26.9 | 34.5 | 28.9 | 26.3 | 29.8 | 26.8 | 27.4 | 32.7 | Sepsis |
| 6062_001 | 29.6 | 31.7 | 25.9 | 23.9 | 29.6 | 27.8 | 27.4 | 26.7 | 27.4 | 33.6 | 28.6 | 24.3 | 28.6 | 26.5 | 25.6 | 31.7 | Sepsis |
| 6065_001 | 28.1 | 28.6 | 26.2 | 24.4 | 28.9 | 30.0 | 26.5 | 26.2 | 26.5 | 34.8 | 27.7 | 24.9 | 29.4 | 26.4 | 25.7 | 32.5 | Sepsis |
| 6070_001 | 28.4 | 29.9 | 26.8 | 25.2 | 27.6 | 28.0 | 26.8 | 26.9 | 26.0 | 34.9 | 28.8 | 25.9 | 29.9 | 27.4 | 26.4 | 32.3 | Sepsis |
| 6073_001 | 29.7 | 31.6 | 26.3 | 24.9 | 29.7 | 29.3 | 28.9 | 26.9 | 25.7 | 33.7 | 29.9 | 25.9 | 29.9 | 27.4 | 27.6 | 32.4 | Sepsis |
| 6075_001 | 31.6 | 33.2 | 24.0 | 23.4 | 32.5 | 27.2 | 28.5 | 26.9 | 27.3 | 34.0 | 27.3 | 24.3 | 27.2 | 26.1 | 26.5 | 32.8 | Sepsis |
| 6078_001 | 27.3 | 30.0 | 24.7 | 24.6 | 28.0 | 27.8 | 26.4 | 25.2 | 26.4 | 31.6 | 28.0 | 24.8 | 28.0 | 26.7 | 26.2 | 32.6 | Sepsis |
| 6081_001 | 30.4 | 30.7 | 27.2 | 24.2 | 30.1 | 29.3 | 29.1 | 27.8 | 27.5 | 33.6 | 30.4 | 26.9 | 29.6 | 29.4 | 28.7 | 33.9 | Sepsis |
| 6082_001 | 30.5 | 32.5 | 27.8 | 26.1 | 29.6 | 30.4 | 28.0 | 28.0 | 28.5 | 33.0 | 29.8 | 27.8 | 30.6 | 28.1 | 26.4 | 31.5 | Sepsis |
| 6084_001 | 28.2 | 27.4 | 25.2 | 24.7 | 27.3 | 28.4 | 26.0 | 26.8 | 25.9 | 27.6 | 32.1 | 25.5 | 29.2 | 29.0 | 32.0 | 24.8 | Sepsis |
| 6085_001 | 29.1 | 30.0 | 27.2 | 24.4 | 28.1 | 28.5 | 27.1 | 26.0 | 26.0 | 33.5 | 29.1 | 25.2 | 29.3 | 27.5 | 26.1 | 32.0 | Sepsis |
| 6098_001 | 28.2 | 29.1 | 26.8 | 24.9 | 25.7 | 28.5 | 26.4 | 25.0 | 25.8 | 32.9 | 27.7 | 24.8 | 29.6 | 26.5 | 24.8 | 32.2 | Sepsis |
| 6104_001 | 28.4 | 27.7 | 26.7 | 23.9 | 26.8 | 27.9 | 26.3 | 24.1 | 25.8 | 25.2 | 31.3 | 25.7 | 28.4 | 29.8 | 32.4 | 25.0 | Sepsis |
| 6110_001 | 28.7 | 31.0 | 26.6 | 24.2 | 27.9 | 27.3 | 28.0 | 26.8 | 26.8 | 33.7 | 28.9 | 26.4 | 28.9 | 27.8 | 26.6 | 33.6 | Sepsis |
| 6115_001 | 30.1 | 31.2 | 28.8 | 24.9 | 27.9 | 29.3 | 26.7 | 26.1 | 27.5 | 34.1 | 29.2 | 27.3 | 31.1 | 28.4 | 26.6 | 32.4 | Sepsis |
| 6125_001 | 28.3 | 29.2 | 26.5 | 23.4 | 26.9 | 27.8 | 25.5 | 24.7 | 26.7 | 32.4 | 28.2 | 25.3 | 29.2 | 27.0 | 25.4 | 31.3 | Sepsis |
| 829_001 | 28.7 | 31.3 | 25.5 | 24.8 | 28.5 | 27.1 | 26.1 | 26.5 | 25.5 | 31.3 | 27.5 | 24.6 | 27.6 | 24.9 | 24.2 | 30.5 | Sepsis |
| 942_001 | 30.0 | 31.9 | 27.7 | 25.2 | 27.7 | 27.7 | 25.8 | 25.4 | 25.9 | 33.8 | 28.7 | 25.8 | 28.8 | 26.8 | 24.6 | 31.7 | Sepsis |
| 987_001 | 26.7 | 28.2 | 25.8 | 22.5 | 25.5 | 26.0 | 24.9 | 24.6 | 26.0 | 31.9 | 26.7 | 24.2 | 28.6 | 26.2 | 24.2 | 31.7 | Sepsis |

TABLE 7
Ct values of the test data per marker (mean of triple determination,
missing values were recorded as NA and excluded from the analysis) and
group affiliation (last column). The first five samples belonging to healthy
subjects, the others to the patients which were described in Table 4. The
corresponding Experiment-ID is made up of the case number and the sample
ID (introduced with two zeros), an additional “1” indicates a repetition.

| Experiment ID | M2 | M3 | M4 | M5 | M6 | M8 | M9 | M10 | M12 | M13 | M15 | M16 | M17 | R1 | R2 | R3 | Group |
|---|---|---|---|---|---|---|---|---|---|---|---|---|---|---|---|---|---|
| 12A | 27.84 | 28.1 | 27.5 | 26.6 | 26.2 | 24.2 | 24.7 | 22.7 | 23.9 | 31.4 | 27.3 | 27.8 | 28.8 | 25.7 | 24.2 | 30.2 | No sepsis |
| 2A | 26.72 | 28.2 | 27.3 | 27.7 | 26.7 | 24.5 | 24.4 | 22.5 | 23.6 | 32.6 | 26.9 | 27.9 | 29.4 | 26.5 | 24.9 | 31.1 | No sepsis |
| 2C | 28.18 | 28.4 | 27.8 | 26.0 | 27.0 | 26.2 | 24.3 | 25.1 | 24.9 | 31.1 | 27.2 | 28.2 | 29.4 | 26.3 | 24.8 | 31.6 | No sepsis |
| 7A | 27.31 | 27.6 | 27.4 | 26.1 | 25.3 | 24.9 | 24.0 | 22.9 | 22.8 | 30.2 | 26.4 | 27.5 | 28.7 | 25.2 | 24.1 | 30.8 | No sepsis |
| 7C | 27.94 | 27.6 | 27.3 | 25.4 | 25.4 | 25.5 | 24.0 | 23.0 | 23.9 | 30.8 | 26.1 | 27.2 | 28.7 | 25.0 | 23.8 | 30.2 | No sepsis |
| 8011_001_1 | 23.06 | 23.8 | 23.5 | 21.3 | 22.3 | 26.9 | 21.3 | 18.7 | 22.8 | 26.6 | 23.8 | 22.5 | 25.2 | 22.6 | 21.4 | 28.0 | No sepsis |
| 8034_001_1 | 25.82 | 26.6 | 25.5 | 23.8 | 25.7 | 26.7 | 24.3 | 24.7 | 25.7 | 31.5 | 25.7 | 24.4 | 28.4 | 25.2 | 24.6 | 30.8 | No sepsis |
| 8044_001_1 | 24.31 | 24.4 | 23.5 | 22.5 | 23.9 | 25.1 | 22.0 | 22.9 | 23.8 | 28.6 | 23.2 | 22.4 | 25.5 | 23.1 | 23.0 | 29.5 | No sepsis |
| 8052_001_1 | 26.64 | 27.2 | 27.1 | 23.6 | 25.3 | 26.7 | 24.3 | 24.0 | 24.9 | 32.2 | 26.3 | 25.2 | 28.0 | 26.0 | 25.0 | 31.6 | No sepsis |
| 8073_001_1 | 25.93 | 27.1 | 26.9 | 25.3 | 25.5 | 27.4 | 25.3 | 23.8 | 24.7 | 30.5 | 27.1 | 25.6 | 28.6 | 27.1 | 25.8 | 31.4 | No sepsis |
| 8103_001_1 | 23.68 | 23.9 | 22.7 | 21.4 | 21.7 | 23.2 | 22.1 | 19.9 | 21.7 | 27.9 | 21.3 | 21.6 | 26.0 | 23.3 | 18.9 | 27.3 | No sepsis |
| 8108_001_1 | 25.79 | 25.5 | 24.0 | 21.0 | 24.4 | 26.5 | 23.4 | 22.5 | 24.1 | 30.1 | 25.6 | 23.2 | 26.1 | 24.4 | 23.4 | 31.3 | No sepsis |
| 5019_001 | 27.41 | 28.0 | 27.2 | 25.5 | 26.3 | 25.5 | 24.6 | 24.4 | 24.5 | 32.4 | 27.2 | 26.0 | 28.8 | 26.5 | 25.5 | 32.0 | No sepsis |
| 5020_001 | 23.28 | 25.3 | 25.4 | 24.3 | 24.0 | 24.7 | 23.1 | 22.3 | 23.1 | 27.4 | 25.1 | 23.9 | 27.0 | 24.7 | 23.5 | 27.8 | No sepsis |
| 5023_001 | 25.14 | 25.5 | 25.4 | 25.1 | 24.6 | 25.0 | 22.9 | 22.8 | 23.3 | 31.6 | 26.0 | 23.3 | 27.5 | 24.5 | 24.0 | 30.2 | No sepsis |
| 8026_001 | 27.06 | 27.8 | 27.5 | 24.4 | 26.3 | 28.6 | 24.5 | 24.1 | 26.3 | 31.8 | 27.1 | 26.0 | 27.9 | 26.8 | 25.7 | 32.0 | No sepsis |
| 8056_002 | 26.30 | 28.4 | 25.9 | 25.2 | 26.3 | 27.7 | 25.5 | 23.9 | 26.1 | 31.4 | 27.3 | 24.5 | 27.7 | 25.6 | 26.0 | 31.8 | keine Sepsis |
| 8058_002 | 27.73 | 29.7 | 27.7 | 24.9 | 26.2 | 28.0 | 24.7 | 23.5 | 25.3 | 32.7 | 27.5 | 24.7 | 29.3 | 26.4 | 24.4 | 30.7 | No sepsis |
| 8086_001 | 25.46 | 25.9 | 25.5 | 24.1 | 24.4 | 26.2 | 23.1 | 22.9 | 23.7 | 30.7 | 25.1 | 24.1 | 27.6 | 24.9 | 24.0 | 30.2 | No sepsis |
| 8122_003 | 27.51 | 28.3 | 26.0 | 22.3 | 25.3 | 26.0 | 23.6 | 23.8 | 24.6 | 31.5 | 25.4 | 25.4 | 27.2 | 25.0 | 24.2 | 30.0 | No sepsis |
| 2042_001 | 28.20 | 31.6 | 28.5 | 24.3 | 28.3 | 30.3 | 26.8 | 25.4 | 27.8 | 34.1 | 29.1 | 25.8 | 29.9 | 29.1 | 26.4 | 33.4 | No sepsis |
| 8102_001 | 27.30 | 29.7 | 27.8 | 24.9 | 26.9 | 28.9 | 24.8 | 23.4 | 26.7 | 33.0 | 28.1 | 26.2 | 30.1 | 26.9 | 25.5 | 31.7 | No sepsis |
| 8111_001 | 25.79 | 27.0 | 26.7 | 23.7 | 25.0 | 27.0 | 23.9 | 24.8 | 26.0 | 32.0 | 26.5 | 24.5 | 29.1 | 26.0 | 24.4 | 31.4 | No sepsis |
| 8112_001 | 24.47 | 24.8 | 23.5 | 21.9 | 24.7 | 26.1 | 22.6 | 22.8 | 22.9 | 30.1 | 23.3 | 22.8 | 28.3 | 22.0 | 21.7 | 29.4 | No sepsis |
| 8116_001 | 26.60 | 30.1 | 27.8 | 24.6 | 26.7 | 28.7 | 25.8 | 24.5 | 26.6 | 32.8 | 28.1 | 25.9 | 29.8 | 27.0 | 25.9 | 32.1 | No sepsis |
| 8039_001 | 25.09 | 27.1 | 25.8 | 23.2 | 24.6 | 25.2 | 22.6 | 23.1 | 23.8 | 31.2 | 25.6 | 26.1 | 27.8 | 25.3 | 23.8 | 29.9 | No sepsis |
| 8039_002 | 25.19 | 26.7 | 24.9 | 23.1 | 24.1 | 28.7 | 22.9 | 26.3 | 22.9 | 30.1 | 25.1 | 25.6 | 27.8 | 24.7 | 23.5 | 30.3 | No sepsis |
| 8039_003 | 26.72 | 27.7 | 26.1 | 24.1 | 25.1 | 25.5 | 24.0 | 24.3 | 25.1 | 32.1 | 27.2 | 25.6 | 28.6 | 26.2 | 25.1 | 30.8 | No sepsis |
| 8039_004 | 27.61 | 30.2 | 26.3 | 24.3 | 26.5 | 30.6 | 24.4 | 27.6 | 25.4 | 33.2 | 27.3 | 26.5 | 28.7 | 26.3 | 25.2 | 31.3 | No sepsis |
| 8039_005 | 26.85 | 25.9 | 25.7 | 25.7 | 24.9 | 27.2 | 25.2 | 25.3 | 25.8 | 30.7 | 26.1 | 25.8 | 27.5 | 25.2 | 24.1 | 30.7 | No sepsis |
| 7052_001 | 28.40 | 29.9 | 26.8 | 24.2 | 27.7 | 26.6 | 25.7 | 24.3 | 25.0 | 34.3 | 28.0 | 26.5 | 28.6 | 26.6 | 25.8 | 31.2 | No sepsis |
| 7052_002 | 29.62 | 31.3 | 27.1 | 24.0 | 29.0 | 27.2 | 26.4 | 25.3 | 26.2 | 33.2 | 29.0 | 26.2 | 29.8 | 26.9 | 25.9 | 31.7 | No sepsis |
| 7052_003 | 29.62 | 31.0 | 28.1 | 24.2 | 29.0 | 27.4 | 26.7 | 26.0 | 27.2 | 33.4 | 29.0 | 27.8 | 29.5 | 28.3 | 26.8 | 32.7 | No sepsis |
| 7119_001 | 26.68 | 27.4 | 26.5 | 24.2 | 25.2 | 26.1 | 24.5 | 23.3 | 24.4 | 31.2 | 26.2 | 24.5 | 28.4 | 25.9 | 23.8 | 31.5 | No sepsis |
| 7119_002 | 27.97 | 29.1 | 28.6 | 25.1 | 27.2 | 26.6 | 26.0 | 24.7 | 26.0 | 32.6 | 27.7 | 26.3 | 29.1 | 26.9 | 25.9 | 32.1 | No sepsis |
| 7119_003 | 28.56 | 29.1 | 28.3 | 24.8 | 26.8 | 26.7 | 25.6 | 24.7 | 25.3 | 31.8 | 27.6 | 27.1 | 29.4 | 26.6 | 25.7 | 30.7 | No sepsis |
| 8026_001_1 | 28.10 | 28.8 | 33.3 | 25.0 | 26.8 | 28.2 | 25.6 | 24.4 | 26.2 | 32.4 | 27.5 | 26.6 | 28.4 | 27.0 | 26.3 | 33.4 | No sepsis |
| 8026_002 | 29.11 | 29.6 | 32.7 | 25.1 | 27.5 | 28.6 | 26.0 | 25.2 | 26.1 | 32.9 | 27.8 | 26.1 | 29.0 | 27.1 | 25.7 | 32.5 | No sepsis |
| 8026_003 | 32.38 | 33.0 | 35.0 | 26.2 | 29.9 | 30.5 | 27.4 | 26.4 | 27.7 | 34.9 | 29.3 | 28.5 | 30.0 | 31.6 | 28.1 | 34.4 | No sepsis |
| 8026_004 | 29.79 | 30.5 | 32.2 | 25.4 | 28.7 | 28.9 | 27.7 | 26.8 | 27.0 | NA | 29.1 | 28.1 | 29.8 | 28.5 | 26.9 | 32.5 | keine Sepsis |
| 8026_005 | 28.06 | 28.6 | 32.0 | 24.5 | 26.1 | 27.0 | 25.3 | 24.0 | 24.9 | 32.5 | 27.4 | 26.7 | 24.9 | 25.7 | 24.8 | 31.5 | No sepsis |
| 7077_001 | 27.22 | 28.8 | 27.3 | 24.1 | 27.8 | 27.1 | 26.5 | 25.6 | 25.8 | 32.6 | 28.1 | 25.9 | 29.8 | 27.1 | 25.6 | 31.5 | No sepsis |
| 7077_002 | 25.50 | 27.6 | 25.8 | 23.0 | 26.2 | 25.5 | 25.3 | 24.5 | 25.3 | 31.5 | 27.4 | 25.2 | 28.4 | 25.1 | 24.8 | 30.4 | Sepsis |
| 7077_003 | 26.50 | 28.4 | 27.5 | 24.3 | 27.8 | 27.1 | 26.0 | 25.4 | 26.1 | 32.1 | 27.4 | 26.6 | 29.4 | 26.6 | 25.2 | 30.7 | Sepsis |
| 7084_001 | 26.16 | 28.2 | 27.0 | 23.6 | 26.1 | 27.4 | 25.3 | 24.2 | 27.0 | 33.3 | 26.8 | 24.7 | 29.9 | 26.0 | 25.5 | 32.0 | No sepsis |
| 7084_002 | 26.19 | 27.7 | 26.2 | 22.6 | 26.6 | 27.0 | 25.8 | 24.1 | 25.1 | 32.2 | 25.8 | 23.5 | 29.1 | 25.7 | 24.0 | 31.2 | No sepsis |
| 7084_003 | 27.42 | 30.0 | 28.2 | 23.9 | 28.6 | 28.5 | 27.0 | 25.6 | 26.2 | 32.9 | 26.9 | 24.6 | 30.3 | 26.5 | 25.1 | 31.5 | Sepsis |
| 7084_004 | 26.61 | 29.9 | 25.8 | 23.9 | 27.9 | 28.2 | 27.2 | 25.4 | 25.5 | 33.0 | 26.3 | 23.6 | 29.7 | 26.8 | 25.7 | 32.2 | Sepsis |
| 7096_001 | 25.59 | 27.0 | 26.9 | 23.2 | 25.3 | 26.0 | 23.7 | 23.3 | 24.6 | 31.4 | 26.7 | 25.4 | 28.7 | 25.8 | 23.8 | 29.9 | No sepsis |
| 7096_002 | 26.40 | 28.4 | 26.9 | 23.6 | 24.8 | 29.7 | 24.5 | 23.7 | 24.6 | 31.0 | 27.6 | 26.2 | 28.2 | 25.8 | 24.4 | 30.9 | No sepsis |
| 7096_003 | 27.50 | 29.5 | 27.6 | 23.8 | 26.0 | 26.4 | 24.6 | 23.9 | 25.1 | 32.5 | 27.8 | 26.7 | 28.9 | 26.5 | 25.0 | 31.2 | No sepsis |
| 7096_004 | 25.80 | 27.9 | 25.8 | 23.6 | 25.8 | 29.8 | 24.2 | 23.9 | 25.1 | 30.7 | 26.5 | 24.5 | 28.5 | 25.2 | 24.5 | 30.6 | No sepsis |
| 7096_005 | 26.01 | 27.7 | 26.3 | 23.2 | 25.2 | 26.1 | 24.1 | 23.8 | 24.0 | 31.7 | 26.3 | 24.5 | 29.1 | 25.6 | 23.6 | 30.3 | No sepsis |
| 7096_006 | 27.14 | 28.5 | 26.9 | 24.2 | 26.8 | 31.2 | 25.4 | 24.7 | 25.0 | 32.4 | 27.8 | 25.5 | 29.6 | 26.5 | 25.2 | 31.1 | Sepsis |
| 7096_007 | 25.97 | 27.5 | 24.9 | 22.7 | 25.3 | 25.6 | 24.3 | 23.9 | 24.6 | 31.3 | 26.5 | 23.8 | 28.4 | 25.0 | 23.8 | 30.2 | Sepsis |
| 8009_001_1 | 27.01 | 27.5 | 31.7 | 25.0 | 26.5 | 26.9 | 24.0 | 23.8 | 24.3 | 32.4 | 27.0 | 26.1 | 28.0 | 26.0 | 25.6 | 31.6 | No sepsis |
| 8009_002 | 25.06 | 26.4 | 24.9 | 22.9 | 24.7 | NA | 22.9 | 22.1 | 23.9 | 31.4 | 26.2 | 23.7 | 26.9 | 24.6 | 23.5 | 31.0 | No sepsis |
| 8009_003 | 24.75 | 24.6 | 21.8 | 20.7 | 24.6 | NA | 23.1 | 22.5 | 22.1 | 29.7 | 23.5 | 20.6 | 24.3 | 22.7 | 21.2 | 28.7 | No sepsis |
| 8009_004 | 27.57 | 28.9 | 29.6 | 23.6 | 26.8 | 27.2 | 25.7 | 23.8 | 23.7 | 33.1 | 26.8 | 23.4 | 27.8 | 26.0 | 24.4 | 31.8 | No sepsis |
| 8009_005 | 28.02 | 28.1 | 30.2 | 24.6 | 27.5 | 27.4 | 25.5 | 24.7 | 25.0 | 33.7 | 27.0 | 24.0 | 27.8 | 25.9 | 24.5 | 31.1 | No sepsis |
| 8009_006 | 27.17 | 28.1 | 28.9 | 24.6 | 26.5 | 26.3 | 25.6 | 24.0 | 24.9 | 32.2 | 26.5 | 23.3 | 27.5 | 25.3 | 23.9 | 30.7 | No sepsis |
| 8009_007 | 26.97 | 28.5 | 28.9 | 23.8 | 27.6 | 25.8 | 25.8 | 23.9 | 24.7 | 33.1 | 26.5 | 23.4 | 28.2 | 25.1 | 23.9 | 30.3 | No sepsis |
| 8009_010 | 29.35 | 29.7 | 30.3 | 25.8 | 28.6 | 28.1 | 26.6 | 25.7 | 26.8 | 34.4 | 27.8 | 24.9 | 29.1 | 26.6 | 25.4 | 32.8 | Sepsis |
| 8096_001_1 | 28.75 | 30.6 | 26.1 | 25.0 | 28.7 | 27.2 | 27.5 | 26.2 | 25.7 | 35.8 | 28.2 | 25.4 | 29.2 | 27.0 | 25.8 | 31.6 | No sepsis |
| 8096_002 | 27.69 | 28.8 | 25.8 | 24.2 | 27.3 | 26.6 | 25.6 | 24.4 | 24.0 | 32.7 | 27.7 | 24.8 | 28.3 | 26.2 | 26.0 | 31.6 | No sepsis |
| 8096_003 | 28.48 | 31.4 | 27.0 | 23.1 | 28.0 | 26.1 | 26.5 | 26.2 | 24.5 | 33.2 | 28.8 | 26.6 | 28.8 | 26.4 | 25.6 | 31.2 | No sepsis |
| 8112_001_1 | 27.42 | 28.7 | 27.0 | 25.3 | 27.3 | 30.1 | 26.5 | 27.2 | 26.7 | 32.6 | 27.5 | 25.9 | 30.0 | 26.4 | 25.6 | 32.8 | No sepsis |
| 8112_002_1 | 27.36 | 28.9 | 26.8 | 25.4 | 26.6 | 31.7 | 26.3 | 26.7 | 26.5 | 32.7 | 27.4 | 25.2 | 29.4 | 26.5 | 25.2 | 32.3 | No sepsis |
| 8112_003 | 27.18 | 28.8 | 27.3 | 24.2 | 26.5 | 29.1 | 26.2 | 26.3 | 24.7 | 32.3 | 27.4 | 25.1 | 29.6 | 26.2 | 24.8 | 31.9 | No sepsis |
| 8112_004 | 27.83 | 29.8 | 27.7 | 25.9 | 26.9 | 32.0 | 27.0 | 26.7 | 25.5 | 33.1 | 28.0 | 25.9 | 29.0 | 26.6 | 24.9 | 31.4 | No sepsis |
| 8112_005 | 28.19 | 30.0 | 27.4 | 24.8 | 26.9 | 30.3 | 26.4 | 26.7 | 25.8 | 34.1 | 27.9 | 26.2 | 29.3 | 26.4 | 24.7 | 31.2 | Sepsis |
| 8112_006 | 28.64 | 30.2 | 27.7 | 26.0 | 27.6 | 32.2 | 26.9 | 27.1 | 26.4 | 34.2 | 28.0 | 26.9 | 30.3 | 27.1 | 25.8 | 32.7 | Sepsis |
| 8101_001 | 24.47 | 25.9 | 24.7 | 21.9 | 24.3 | 24.5 | 23.0 | 22.5 | 25.1 | 30.6 | 25.1 | 23.3 | 26.5 | 24.2 | 23.3 | 30.6 | No sepsis |
| 8101_002 | 24.87 | 27.0 | 25.3 | 22.8 | 25.2 | 24.8 | 23.9 | 23.2 | 24.0 | 30.8 | 25.6 | 23.7 | 26.7 | 24.5 | 23.5 | 17.6 | No sepsis |
| 8101_003 | 24.48 | 25.7 | 23.7 | 22.2 | 24.2 | 29.5 | 23.7 | 23.5 | 24.8 | 31.1 | 25.0 | 22.3 | 26.4 | 24.0 | 23.2 | 31.0 | No sepsis |
| 8101_004 | 24.88 | 26.9 | 24.1 | 22.3 | 24.6 | 24.9 | 24.2 | 23.7 | 23.6 | 30.2 | 24.9 | 22.2 | 25.7 | 24.1 | 22.6 | 29.2 | Sepsis |
| 8101_005 | 23.84 | 26.4 | 22.4 | 22.6 | 24.9 | 24.2 | 23.9 | 22.8 | 24.9 | 28.9 | 24.6 | 22.4 | 25.0 | 24.1 | 22.9 | 28.7 | Sepsis |
| 8111_003 | 25.77 | 26.7 | 24.8 | 23.1 | 24.9 | 24.9 | 24.2 | 24.0 | 26.1 | 31.0 | 25.3 | 23.3 | 27.3 | 24.4 | 23.1 | 30.1 | No sepsis |
| 8111_004 | 25.15 | 26.7 | 26.0 | 22.5 | 24.1 | 24.4 | 23.6 | 24.2 | 25.5 | 30.1 | 25.6 | 23.5 | 27.3 | 25.6 | 24.4 | 30.0 | No sepsis |
| 8111_005 | 25.32 | 26.6 | 26.8 | 22.7 | 24.7 | 25.1 | 23.6 | 23.2 | 24.2 | 30.4 | 25.8 | 24.4 | 27.5 | 25.1 | 24.3 | 31.4 | No sepsis |
| 8111_006 | 26.53 | 26.9 | 25.3 | 23.2 | 25.9 | 25.3 | 24.7 | 25.7 | 26.2 | 31.4 | 25.7 | 24.1 | 28.3 | 24.6 | 24.8 | 30.6 | Sepsis |
| 8111_007 | 25.88 | 26.8 | 26.5 | 23.6 | 25.8 | 26.5 | 25.6 | 26.0 | 27.1 | 29.4 | 26.4 | 23.6 | 28.4 | 26.0 | 24.1 | 28.5 | Sepsis |
| 6008_002 | 25.89 | 26.1 | 22.4 | 20.5 | 26.0 | 25.6 | 24.5 | 22.5 | 22.8 | 28.4 | 23.4 | 21.9 | 28.2 | 23.0 | 22.6 | 28.1 | Sepsis |
| 6063_001 | 26.45 | 26.8 | 25.0 | 23.0 | 25.6 | 25.7 | 24.3 | 23.3 | 25.0 | 30.9 | 25.9 | 23.5 | 28.3 | 25.2 | 23.6 | 30.0 | Sepsis |
| 6141_001 | 27.23 | 29.9 | 25.6 | 23.9 | 28.0 | 27.9 | 26.4 | 26.0 | 26.6 | 31.8 | 27.2 | 24.1 | 27.9 | 25.2 | 25.0 | 31.6 | Sepsis |
| 1013_001 | 28.86 | 30.9 | 26.9 | 23.6 | 28.4 | 27.1 | 26.0 | 25.6 | 26.3 | 33.3 | 28.1 | 25.0 | 25.5 | 26.6 | 24.6 | 31.6 | Sepsis |
| 6024_002 | 26.64 | 28.5 | 25.3 | 21.9 | 26.1 | 24.8 | 24.7 | 23.9 | 23.7 | 32.2 | 26.0 | 24.7 | 28.3 | 25.6 | 25.1 | 31.2 | Sepsis |
| 7040_001 | 28.55 | 31.1 | 26.1 | 23.7 | 28.4 | 26.3 | 26.8 | 25.7 | 26.1 | 33.9 | 27.6 | 24.7 | 28.5 | 26.1 | 25.0 | 31.5 | Sepsis |
| 7079_002 | 27.32 | 30.4 | 26.3 | 23.6 | 28.6 | 26.6 | 27.1 | 26.5 | 25.8 | 30.7 | 27.2 | 25.3 | 28.6 | 26.4 | 25.0 | 29.8 | Sepsis |
| 920_001 | 29.50 | 31.4 | 29.1 | 26.0 | 28.2 | 27.5 | 26.6 | 25.1 | 25.8 | 32.7 | 28.6 | 27.0 | 29.1 | 27.4 | 25.5 | 30.7 | Sepsis |
| 6036_001 | 28.82 | 30.0 | 26.8 | 24.6 | 28.7 | 26.5 | 27.1 | 25.3 | 24.0 | 34.7 | 27.7 | 26.7 | 29.5 | 26.8 | 25.9 | 31.3 | Sepsis |
| 6056_001 | 26.28 | 27.9 | 25.4 | 24.6 | 27.6 | 26.0 | 25.5 | 25.3 | 27.2 | 31.0 | 26.5 | 25.5 | 27.5 | 25.7 | 24.8 | 30.1 | Sepsis |
| 6061_001 | 28.63 | 29.0 | 27.0 | 25.2 | 28.2 | 26.5 | 26.3 | 25.6 | 26.2 | 32.6 | 26.7 | 25.5 | 30.0 | 26.4 | 25.0 | 30.8 | Sepsis |
| 7023_001 | 26.83 | 29.2 | 26.5 | 24.3 | 26.5 | 25.5 | 25.7 | 24.5 | 25.5 | 34.7 | 26.3 | 25.9 | 28.6 | 27.4 | 25.7 | 31.7 | Sepsis |
| 7112_001 | 28.60 | 29.5 | 31.3 | 25.9 | 27.9 | 27.9 | 25.4 | 26.3 | 25.3 | 33.8 | 27.0 | 24.7 | 30.1 | 26.1 | 24.9 | 31.6 | Sepsis |
| 6064_001 | 28.63 | 30.0 | 30.0 | 22.8 | 29.6 | 26.2 | 26.2 | 26.2 | 26.1 | 33.8 | 27.1 | 24.3 | 28.9 | 25.6 | 25.1 | 30.6 | Sepsis |
| 6120_001 | 27.58 | 30.7 | 25.7 | 23.4 | 29.0 | 27.8 | 27.2 | 27.3 | 27.2 | 33.7 | 28.1 | 24.6 | 28.6 | 26.4 | 25.3 | 31.4 | Sepsis |
| 7105_001 | 27.19 | 28.6 | 32.3 | 22.7 | 26.6 | 26.4 | 25.1 | 25.1 | 24.6 | 31.7 | 27.7 | 24.4 | 29.1 | 26.0 | 25.2 | 30.8 | Sepsis |
| 7120_001 | 29.38 | 29.8 | 29.6 | 24.4 | 29.3 | 28.2 | 26.9 | 26.3 | 26.2 | 33.5 | 27.9 | 24.7 | 28.2 | 26.6 | 25.7 | 32.2 | Sepsis |
| 749_001 | 27.59 | 28.8 | 26.4 | 23.5 | 26.5 | 25.6 | 24.0 | 25.1 | 25.3 | 31.2 | 26.3 | 25.1 | 28.6 | 25.1 | 24.1 | 30.5 | Sepsis |
| 5008_001 | 26.87 | 27.9 | 31.2 | 22.0 | 26.5 | 26.1 | 24.5 | 24.0 | 25.5 | 32.3 | 27.2 | 24.8 | 28.3 | 25.6 | 24.3 | 30.8 | Sepsis |
| 5009_001 | 25.52 | 26.2 | 29.7 | 22.3 | 25.9 | 25.5 | 23.2 | 24.2 | 26.3 | 30.8 | 26.0 | 24.2 | 27.4 | 25.0 | 23.4 | 29.0 | Sepsis |
| 5010_001 | 27.96 | 29.1 | 29.5 | 22.8 | 28.8 | 27.4 | 28.5 | 26.3 | 27.0 | 32.6 | 27.9 | 25.6 | 27.8 | 26.1 | 26.7 | 31.9 | Sepsis |
| 5018_001 | 28.47 | 30.4 | 30.0 | 23.7 | 28.4 | 27.6 | 26.8 | 26.6 | 26.7 | 34.0 | 27.3 | 25.3 | 29.2 | 26.2 | 25.9 | 31.9 | Sepsis |
| 1015_001 | 29.77 | 30.5 | 26.7 | 24.8 | 28.8 | 28.3 | 26.7 | 26.1 | 26.8 | 34.5 | 28.3 | 25.0 | 29.3 | 26.9 | 25.5 | 30.5 | Sepsis |
| 6005_001 | 29.27 | 30.0 | 26.9 | 23.5 | 28.3 | 26.4 | 26.8 | 24.9 | 21.6 | 33.4 | 27.7 | 24.9 | 28.8 | 26.2 | 25.6 | 32.1 | Sepsis |
| 6035_002 | 28.80 | 28.0 | 26.3 | 25.0 | 28.0 | 27.4 | 26.9 | 26.2 | 25.7 | 32.8 | 27.1 | 25.6 | 29.1 | 26.6 | 25.3 | 31.6 | Sepsis |
| 6070_002 | 29.09 | 31.0 | 27.7 | 24.7 | 28.2 | 28.9 | 26.9 | 25.8 | 27.4 | 34.1 | 29.8 | 26.5 | 29.8 | 28.1 | 26.4 | 33.0 | Sepsis |
| 6075_002 | 29.35 | 30.6 | 25.6 | 25.8 | 28.9 | 28.7 | 26.8 | 26.4 | 27.2 | 32.6 | 28.0 | 24.6 | 28.5 | 26.3 | 25.4 | 31.6 | Sepsis |
| 6104_002 | 30.63 | 32.3 | 28.8 | 25.8 | 28.5 | 28.5 | 27.2 | 27.2 | 26.8 | 34.4 | 28.9 | 27.2 | 29.0 | 27.5 | 26.3 | 32.1 | Sepsis |
| 6124_001 | 30.50 | 31.3 | 26.7 | 24.8 | 29.1 | 26.7 | 28.1 | 25.0 | 26.1 | 32.7 | 28.3 | 26.0 | 28.5 | 27.0 | 26.4 | 31.9 | Sepsis |
| 6126_001 | 30.72 | 28.6 | 27.6 | 24.5 | 27.8 | 26.2 | 26.0 | 24.7 | 24.5 | 31.4 | 26.8 | 25.7 | 26.9 | 25.9 | 24.7 | 30.4 | Sepsis |
| 714_001 | 32.80 | 31.9 | 27.6 | 27.5 | 29.9 | 30.3 | 29.8 | 28.0 | 25.2 | NA | 29.5 | 26.8 | 31.6 | 28.9 | 28.0 | 33.5 | Sepsis |

### Classification:

The aim of classification was to determine the marker sets, which allow the best separation between samples from patients with and without sepsis. In order to arrange the gene-markers according to ability to differentiate, a linear discriminant analysis (LDA) [Hastie et al. 2001] was used together with the method of forward selection, wherein the ability to discriminate was assessed with the F-value [Hocking, R. R., 1976].

The calculation was performed by using the function lda of the R-Library MASS. For p markers the weights (w₀, . . . , wₚ) of the discriminant function ƒ_{LD}, by the formula ƒ_{LD} were calculated from the training data set using the formula

$${f}_{LD} ( {x}_{1} , … , {x}_{p} ) = {\sum }_{i = 1}^{p} {w}_{i} {x}_{i} - {w}_{0}$$

Each training sample was subsequently classified, wherein the delta Ct values of the samples were used for xᵢ in the above formula. The weights of the discriminant function were calculated such that a positive value of the function implies an assignment to the group with infectious complications and a negative value of the function implies assignment to the group without an infectious complication.

The classification procedure was repeated for an increasing number of markers, and gradually those marker candidates were included in discriminant analysis which had the highest contribution if distinguishing ability (forward selection). This analysis step was repeated for 1,000 bootstrap samples, which were obtained by random sampling with putting back from the training data set. The marker rank determined in each repeat was averaged over 1,000 runs. The marker candidates were arranged in ascending order according to averaged rank. This arrangement means that the marker with the smallest mean rank is the one who provided the greatest contribution to the discrimination quality and the marker with the highest mean rank was the one who contributed, in most repetitions, little towards the discrimination.

The quality of the identified markers rankings was checked, in that the distinguishability of the training groups for marker sets was evaluated in with increasing of marker numbers. For this, linear discriminant analysis was used with a simple cross-validation. For p markers the weights(w₀, . . . , wₚ) of the discriminant function ƒ_{LD}, by the formula ƒ_{LD} were calculated from the reduced training data, of the in which for each sequential run a sample was omitted. This sample was subsequently classified, for which the previous formula for xᵢ the delta Ct values of the samples were used.

To verify that separation qualities are not primary due to the classification methods but rather depend on selection of the marker, subsequently a simple cross-validation was repeated in the same way, also for the quadratic discriminant analysis (QDA) [Hastie et al. 2001]. The calculation was based on the use of QDA function from the R-Library MASS.

The classification results of the training set were validated for the matrix of test data. From the training data set, for each marker set with increasing marker number the discriminant function was determined and used for classification of test samples. The quality of the classification was measured using the Receiver Operating Characteristic (ROC)-curve, in which the rate of true positives (sensitivity) is shown against the rate of false positives (1-specificity) for an increasing sequence of classification thresholds [Fawcett T., 2006]. For the assessment the area under curve (AUC) was calculated for each ROC curve and the highest attainable classification efficiency (percentage of correctly classified samples) was determined.

### Results

The results of the above-described classification analysis were summarized in Table 8. The ranking procedure resulted in the following order of the marker candidates: M6, M15, M9, M7, M2, M10, M4, M12, M17, M3, M8, M13, M16. The cross-validation rate of the LDA and the QDA increased significantly for the first three markers to 94.8%. The best separation of the training groups at 96.6% was achieved with LDA for the first six markers, in which 56 of 58 samples were classified correctly in (QDA provides a maximum with 3 markers followed by 7 markers). For more than 7 markers, no improvement in classification was achieved.

For the independent test data set the largest area under the ROC curve of more than 85% was achieved for the first 6 and 7 markers, the best classification at 81.4% was achieved with the first 7 markers.

TABLE 8
Results of classification optimization. Indicated in bold is the
maximum value of the respective column, which reflects the best
result with respect to the marker combination.

| | Training data (n = 58) | | | | |
|---|---|---|---|---|---|
| | | Cross | | | |
| | | validation | | Test data (n = 113) | |
| | Middle rank | date | | Area under the | |
| Marker- | (1000 | (%) | | ROC curve, AUC | Classification |
| ranking | Bootstraps) | LDA | QDA | (%) | efficiency (%) |
| M6 | 3.3 | 70.7 | 74.1 | 60.1 | 60.2 |
| M15 | 4.1 | 79.3 | 81.0 | 68.1 | 68.1 |
| M9 | 4.4 | 94.8 | 94.8 | 84.0 | 78.8 |
| M7 | 5.7 | 93.1 | 87.9 | 84.3 | 77.9 |
| M2 | 5.7 | 93.1 | 89.7 | 83.8 | 75.2 |
| M10 | 7.0 | 96.6 | 87.9 | 85.2 | 78.8 |
| M4 | 7.1 | 91.4 | 93.1 | 85.4 | 81.4 |
| M12 | 7.2 | 89.7 | 89.7 | 83.9 | 78.8 |
| M17 | 8.7 | 91.4 | 89.7 | 82.5 | 77.9 |
| M3 | 8.9 | 91.4 | 87.9 | 81.9 | 78.8 |
| M8 | 9.4 | 89.7 | 84.5 | 80.3 | 75.2 |
| M13 | 9.4 | 87.9 | 81.0 | 79.2 | 73.5 |
| M16 | 10.3 | 87.9 | 77.6 | 78.4 | 73.5 |

Since the best results were mainly achieved with the first seven markers in the above table, for further classification the 7-marker-LDA was used, of which the discriminant function ƒ_{LD} was determined from the training data set. The corresponding weights (w₀, . . . , wₚ) are shown in Table 9.

Based on this function, a sepsis related diagnostic parameter, a so-called SIQ score (SIQ) was established as follows. For a new independent sample one is given as a classification result a dimension free value of the discriminant function. A positive value classifies the sample as infectious and a negative value as a non-infectious. For typical representatives of each group one obtains higher absolute values, difficult to classify samples have values close to zero. The scatter of the discriminant values is attributable among other things to the variability of the delta Ct-data matrix. Thus one arrives at, in the classification discrimination, values of about −5 to 5. To more pronouncedly illustrate the differences, the SIQ-score (SIQ) is shown as the 10-fold value of the discriminant function with the weights from the Table 9. Accordingly, the values of the SIQ-test data vary from about −50 to 50.

TABLE 9
Weights of the discriminant function, which were obtained from the
training data set.

| | w1 | w2 | w3 | w4 | w5 | w6 | w7 |
|---|---|---|---|---|---|---|---|
| w0 | (M6) | (M15) | (M9) | (M7) | (M2) | (M10) | (M4) |
| 0.160 | 0.733 | −0.722 | −1.006 | 0.188 | −0.387 | −0.268 | 0.161 |

In linear discriminant analysis the discriminant function, among other things, is so calculated or defined, that the separation threshold between the two training groups is nil. Using the ROC curve, it can be determined using an independent test data set, at which threshold value the best separation of the corresponding test groups is achieved. FIG. 1 shows the ROC curve for classification of test data using the SIQ score is presented and the relationship between true positives (sensitivity) and false positives (1-specificity) is shown, gray dashed lines for the threshold of zero, and black dashed for the best achieved classification of 81.4%. This classification result was reached for the threshold value of SIQ=−4.9. FIG. 1 shows that the displacement of the threshold from 0 to −4.9 a sensitivity gain of about 63% to 80% is achieved, but at the expense of specificity from 81% to about 80%. This result reflects the discrepancy between pre-selected patients in the training set and the heterogeneous group of the test data set. The classification quality, which was achieved with the updated classification threshold of SIQ=−4.9, is shown in Table 10.

This demonstrates that the described invention can be used diagnostically for distinguishing between infectious and non-infectious etiology of systemic reactions of the organism, such as SIRS, sepsis, postoperative complications of chronic and/or acute organ dysfunction, shock response, inflammatory response and/or trauma.

TABLE 10
quality of the classification for the test data set using the
SIQ scores at a threshold

| | | Gold standard | | | |
|---|---|---|---|---|---|
| | | Sepsis | no Sepsis | Sum | Prediction |
| | | (n) | (n) | (n) | values |
| SIQ-Score | positive | 32.7% | 12.4% | 45.1% | 72.5% |
| (threshold = | (n) | (37) | (14) | (51) | |
| −4.9) | negative | 6.2% | 48.7% | 54.9% | 88.7% |
| | (n) | (7) | (55) | (62) | |
| | Sum | 38.9% | 61.1% | 100.0% | |
| | (n) | (44) | (69) | (113) | |
| | | Sensitivity | Specificity | Efficiency | |
| | | 84.1% | 79.7% | 81.4% | |

### Example 2

### Early Detection of Sepsis

In the test data set of the first illustrative embodiment, from Case No. 8112, four samples taken before and two samples taken after the development of sepsis were analyzed (see Tables 4 and 7). In the classification analysis a SIQ-score with a value of −4.9 was already achieved two days before the clinical onset of sepsis. The course of the SIQ score introduced in the first illustrative embodiment as well as the course of further sepsis-related clinical parameters (PCT, CRP, SOFA, body temperature, shock treatment) is shown in FIG. 2. From FIG. 2 it can be seen that SIQ score is the only parameter that reflects early the infectious complications. Therewith, it is demonstrated that the described invention can be applied for the early detection of infectious complications such as sepsis and/or generalized infection.

### Example 3

### Monitoring the Course of Therapy

From the test data set of the first embodiment, Case No. 7084, two samples from before and two samples after the development of sepsis were examined (see Tables 4 and 7). For the third illustrative embodiment five successive samples of this patient were measured and evaluated in the same manner as described in the first example (see Table 11). In FIG. 3 the ascertained values of the SIQ scores imported into the first embodiment are shown together with the relevant clinical parameters of sepsis, i.e., PCT, CRP, SOFA, body temperature and the dosage of catecholamines (norepinephrine), which reflects the shock-treatment. From FIG. 3 it can be seen that SIQ score increased above the threshold to −4.9 the day before the clinical onset of sepsis and remained above the threshold during the acute phase. After clinical onset of sepsis an appropriate antibiotic therapy and a shock treatment was started (see Table 4 and FIG. 3). The acute phase ended with discontinuation of catecholamines (shock treatment) at the eighth day. The improved condition of the patient is also reflected in the fall of the SOFA scores beginning from day 7. After the acute phase, the SIQ score also fell and on the 8th day dropped below the threshold of −4.9. The parameters PCT and CRP decrease also, but remain above the corresponding diagnostic decision value. It is demonstrated that the described invention is useful for the monitoring and/or therapy control of, e.g., antibiotic therapy and/or adjunctive clinical measures and/or operational remedial measures.

TABLE 11
Ct-values of training data per marker (mean of triple determination)
and group affiliation (last column)

| Experiment | | | | | | | | | | | | | | | | | |
|---|---|---|---|---|---|---|---|---|---|---|---|---|---|---|---|---|---|
| ID | M2 | M3 | M4 | M5 | M6 | M8 | M9 | M10 | M12 | M13 | M15 | M16 | M17 | R1 | R2 | R3 | |
| 7084_001 | 26.2 | 28.2 | 27.0 | 23.6 | 26.1 | 27.4 | 25.3 | 24.2 | 27.0 | 33.3 | 26.8 | 24.7 | 29.9 | 26.0 | 25.5 | 32.0 | No |
| | | | | | | | | | | | | | | | | | sepsis |
| 7084_002 | 26.2 | 27.7 | 26.2 | 22.6 | 26.6 | 27.0 | 25.8 | 24.1 | 25.1 | 32.2 | 25.8 | 23.5 | 29.1 | 25.7 | 24.0 | 31.2 | No |
| | | | | | | | | | | | | | | | | | sepsis |
| 7084_003 | 27.4 | 30.0 | 28.2 | 23.9 | 28.6 | 28.5 | 27.0 | 25.6 | 26.2 | 32.9 | 26.9 | 24.6 | 30.3 | 26.5 | 25.1 | 31.5 | Sepsis |
| 7084_004 | 26.6 | 29.9 | 25.8 | 23.9 | 27.9 | 28.2 | 27.2 | 25.4 | 25.5 | 33.0 | 26.3 | 23.6 | 29.7 | 26.8 | 25.7 | 32.2 | Sepsis |
| 7084_005 | 24.8 | 28.3 | 25.1 | 22.8 | 27.2 | 27.6 | 26.4 | 24.6 | 24.1 | 29.5 | 26.1 | 22.4 | 28.4 | 25.7 | 24.1 | 28.7 | Sepsis |
| 7084_006 | 26.6 | 28.9 | 26.0 | 23.5 | 26.7 | 30.9 | 26.5 | 24.8 | 25.3 | 32.1 | 26.6 | 23.0 | 29.5 | 25.9 | 24.7 | 31.8 | Sepsis |
| 7084_007 | 25.4 | 27.5 | 25.2 | 23.7 | 26.7 | 28.0 | 25.0 | 23.8 | 22.1 | 31.5 | 25.7 | 21.9 | 28.8 | 25.0 | 23.3 | 31.7 | Sepsis |
| 7084_008 | 24.8 | 26.7 | 24.4 | 23.0 | 26.4 | 30.5 | 25.4 | 24.3 | 24.0 | 31.1 | 25.2 | 21.5 | 28.9 | 24.6 | 23.5 | 31.5 | Sepsis |
| 7084_009 | 25.9 | 28.0 | 25.8 | 23.7 | 27.4 | 28.3 | 25.4 | 24.3 | 25.9 | 32.3 | 26.8 | 23.6 | 29.3 | 26.3 | 24.6 | 31.7 | Sepsis |

### LITERATURE

ACCP/SCCM (1992), Crit. Care Med 20, 864-74
Alberti, C., Brun-Buisson, C., Goodman, S. V., Guidici, D., Granton, J., Moreno, R., Smithies, M., Thomas, O., Artigas, A., Le Gal, I Jr.; European Sepsis Group (2003) Influence of systemic inflammatory response syndrome and Sepsis on outcome of critically ill infected patients. Am J Respir Crit Care Med 168, 77-84.
Altschul, et al., J. Mol. Biol., 215:403-410; 1990.
Amin, K., Kauffman, C. A., (2003), Fever of unknown origin. Postgrad Med, 114(3), 69-75.
Baker, S. G., and Kramer, B., (2006), Identifying genes that contribute most to good classification in microarray. BMC Bioinformatics 7, 407.
Benson, D. A., Karsch-Mizrachi, I., Lipman, D. J., Ostell, J., Sayers, E. W. GenBank. Nucleic Acids Res., 2009 January; 37(Database issue):D26-31.
Blake, P. G., (2008) Complicated peritonitis—the biggest cause of technique failure. Perit. Dial. Int., 28(4):327-328
Bone, R. C., Balk, R. A., Cerra, F. B., Dellinger, E. P., Fein, A. M., Knaus, W. A., Schein, R. M., Sibbald, W. J., the ACCP/SCCM Consensus Conference Committee (1992) Definitions for Sepsis and organ failure and guidelines for the use of innovative therapies in Sepsis. Chest 101, 1656-1662
Box G E P, Cox D R (1964) An analysis of transformations (with discussion). J. Roy, Stat., Soc., B., 26, 211-252
Buness, A., Huber, W., Steiner, K., Sültmann, H., Poustka, A., (2005) ArrayMagic: two-colour cDNA microarray quality control and preprocessing. Bioinformatics 21, 554-556.
Breiman, L., (2001) Random Forests. Machine Learning 45(1), 5-32
Brun-Buisson, C., Doyon, F., Carlet, J., Dellamonica, P., Gouin, F., Lepoutre, A., Mercier, J. C., Offenstadt, G., Regnier, B., (1995) Incidence, risk factors, and outcome of severe Sepsis and septic shock in adults. A multicenter prospective study in intensive care units. French ICU Group for Severe Sepsis, JAMA 274, 968-974
Brun-Buisson, C., Roudot-Thoraval, F., Girou, E., Grenier-Sennelier, C., Durand-Zaleski, I., (2003) The costs of septic syndromes in the intensive care unit and influence of hospital-acquired Sepsis. Intensive Care Med. 29, 1464-1471
Bustin, S. A., (2002) Quantification of mRNA using real-time reverse transcription PCR(RT-PCR): trends and problems. J. Mol, Endicronol 29, 23-29
Calandra, T., Cohen, J., (2005) International Sepsis Forum Definition of Infection in the ICU Consensus Conference. Critical Care Med., 33(7), 1538-48
Carrigan, S. D., Scott, G., Tabrizian, M., (2004) Toward resolving the challenges of Sepsis. Clin Chem 50(8), 1301-1314
DE 10 2007 036 678 Use of polynucleotides for the detection of gene activity for the distinction between local and systemic infection
DE 102007036678.9 (not published)
Ding B Y and Gentleman R (2004) Classification using generalized partial least squares. Bioconductor Project Working Papers. Paper 5. http://www.begress.com/bioconductor/paper5 9
Efron, B., (1979) Bootstrap Methods: Another Look at the Jackknife. The Annals of Statistics 7(1), 1-26
Fawcett, T. (2006). An introduction to ROC analysis. Pattern Recognition Letters, 27, 861-874.
FDA: In Vitro Diagnostic Multivariate Index Assays. Draft Guidance for Industry, Clinical Laboratories, and FDA Staff (2003) http://www.fda.gov/cdrh/oivd/guidance/1610.pdf
Feezor, R. J., Baker, H. V., Xiao, W., et al. (2004) Genomic and Proteomic Determinants of Outcome in Patients Undergoing Thoracoabdominal Aortic Aneurysm Repair. J. Immun, 172, 7103-7109
Klein, D., (2002) Quantification using real-time PCR technology: applications and limitations. Trends Mol Med., 8(6), 257-260
Koulaouzidis, A, Bhat, S., Saeed, A. A., (2009) Spontaneous bacterial peritonitis. World J Gastroenterol. 15(9):1042-9.
Mayhall. G., (2001) Ventilator-Associated Pneumonia or Not-Contemporary Diagnosis. Emerging Infection Disease CDC 7(2)
Golub, T. R., Slonim, D K., Tamayo, P., et al. (1999) Molecular classification of cancer: class discovery and class prediction by gene expression monitoring. Science 286, 531-537
Hastie, T., Tibshirani, R., Friedman, J., (2001) “The Elements of Statistical Learning”, Springer series in statistics, ISBN 0-387-95284-5.
Hocking, R. R., (1976) “The Analysis and Selection of Variables in Linear Regression” Biometrics, 32.
Hollander, M., Wolfe D., (1973), Nonparametric statistical inference. New York: John Wiley & Sons.
Huber, W., von Heydebreck, A., Sueltmann, H., Poustka, A., Vingron, M., (2003) Parameter estimation for the calibration and variance stabilization of microarray data. Stat. Appl. in Genetics and Mol. Biology 2(1), Article 3
Huggett, J., Dheda, K., Bustin, S., et al. (2005) Real-time RT-PCr normalisation; strategies and considerations Genes Immun, 6(4), 279-284
Increase in National Hospital Discharge Survey rates for septicemia—United States, 1979-1987. MMWR Morb Mortal Wkly Rep 1990 39, 31-34
Johnson, S. B., Lissauer, M., Bochicchio, G. V., Moore, R., Cross, A. S., Scalea, T. M., (2007) Gene Expression Profiles Differentiate Between Sterile SIRS and Early Sepsis Annals of Surgery 245(4), 611-621
Knaus, W. A., Draper, E. A., Wagner, D. P., Zimmermann, J. E., (1985) Prognosis in acute organ-system failure. Ann Surg., 202, 658-693
Kofoed, K., Andersen, O., Kronborg, G., Tvede, M., Petersen, J., Eugen-Olsen, J., Larsen, K., (2007), Use of plasma C-reactive protein, procalcitonin, neutrophils, macrophage migration inhibitory factor, soluble urokinase-type plasminogen activator receptor, and soluble triggering receptor expressed on myeloid cells-1 in combination to diagnose infections: a prospective study. Critical Care, 11, R38
Kubista, M., Andrade, J. M., Bengtsson, M., et al. (2006) The real-time polymerase chain reaction. Mol. Aspects Med., 27, 95-125
Kumar A., Roberts D, Wood K E et al. (2006) Duration of hypotension before initiation of effective antimicrobial therapy is the critical determinant of survival in human septic shock. Crit. Care Med., 34(6), 1589-1596
Le-Gall, J R., Lemeshow, S., Leleu, G., Klar, J., Huillard, J., Rue, M., Teres, D., Artigas A (1995) Customized probability models for early severe Sepsis in adult intensive care patients. Intensive Care Unit Scoring Group. JAMA 273, 644-650
Levy, M. M., Fink, M. P., Marshall, J. C., Abraham, E., Angus, D., Cook, D., Cohen, J., Opal, S. M., Vincent, J. L., Ramsay. G., et al. (2003) 2001 SCCM/ESICM/ACCP/ATS/SIS International Sepsis Definitions Conference. Intensive Care Med., 29, 530-538
Liu, H., Li, J., Wong, L., (2005) Use of extreme patient samples for outcome prediction from gene expression data. Bioinformatics 21(16), 3377-3384
MAQC Consortium. The MicroArray Quality Control (MAQC) project shows inter- and intraplatform reproducibility of gene expression measurements. Nat. Biotechnol 2006, 24, 1151-61
Mathiak, G., Kabir. K, Grass, G., et al. (2003) Lipopolysaccharides from different bacterial sources elicit disparate cytokine responses in whole blood assays. Int. J. Mol. Med., 11(1), 41-44
Marshall, J. C., Vincent, J. L., Fink, M. P. et al. Measures, markers, and mediators: toward a staging system for clinical sepsis. A report of the Fifth Toronto Sepsis Roundtable, Toronto, Ontario, Canada, Oct. 25-26, 2000 and Crit. Care Med., 2003, 31: 1560-1567
Mayhall, C. G., (2001) Ventilator-Associated Pneumonia or Not-Contemporary Diagnosis. Emerg. Infect Dis., 7(2), 200-204
Nolan, T., Hands, R. E., Bustin, S. A., (2006) Quantification of mRNA using real-time RT-PCR. Nat. Protoc., 1(3), 1559-1582
Opal, S. M., Lim, Y-P., Siryaporn, E., et al. (2005) Longitudinal studies of inter-alpha inhibitor proteins in severely septic patients: A potential clinical marker and mediator of severe sepsis. Clin. Invest., 35(2), 387-292
Pachot, A., Lepape, A., Vey, S., et al. (2006) Systemic transcriptional analysis in survivor and non-survivor septic shock patients: a preliminary study. Immunol Lett 106(1), 63-71. Epub., 2006 May 17
Pile, J. C., (2006) Evaluating postoperative fever: a focused approach. Clev. Clin. J. Med., 73 (supp. 1) 62-66
Pruitt, K. D., Tatusova T., Maglott, D. R., NCBI reference sequences (RefSeq): a curated non-redundant sequence database of genomes, transcripts and proteins. Nucleic Acids Res. 2007 January; 35(Database issue):D61-5.
Ramilo, O., Allman, W., Chung, W., Mejias, A., Ardura, M., Glaser, C., Wittkowski, K. M., Piqueras, P., Bancherau, J., Palucka, K. A., Chaussabel, D., (2007) Gene expression patterns in blood leukocytes discriminate patients with acute infections. Blood 109, 2066-2077
R. Development Core Team (2006) R: A language and environment for statistical computing. R Foundation for Statistical Computing, Vienna, Austria. ISBN 3-900051-07-0, URL http://www.R-project.org
Rocke, D. M., Durbin, B., (2001) A model for measurement error for gene expression arrays. J. Comput. Biol., 8, 557-569
Roth, A. R., Basello, D. O., (2003) Approach to the adult patient with fever of unknown origin. Am. Fam. Phys., 68(11) 2223-2228
Ruokonen, E., et al. (1999) Procalcitonin concentrations in patients with neutropenic fever. Eur. J. Clin. Microbiol. Infect. Dis., 18(4), 283-5
Ruokonen, E., et al. (2002) Procalcitonin and neopterin as indicators of infection in critically ill patients. Acta Anaesthesiol Scand 46(4), 398-404
Simon, L., Gauvin, F., Amre, D. K., Saint-Lois, P., Lacroix, J., (2004) Serum procalcitonin and C-reactive protein levels as marker of bacterial infection: A systematic review and meta analysis. Clin. Infec. Dis., 39, 206-217
Simon, R., (2005) Roadmap for Developing and Validating Therapeutically Relevant Genomic Classifiers. J. Clin. Oncol., 23, 7332-7341
Sponholz, C., Sakr, Y., Reinhart, K., Brunkhorst, F., (2006) Diagnostic value and prognostic implications of serum procalcitonin after cardiac surgery: a systematic review of the literature, Critical Care 10, R145
Suprin, E., et al. (2000) Procalcitonin: a valuable indicator of infection in a medical ICU Intensive Care Med., 26(9), 1232-1238
Tang, B. M. P., Eslick, G. D., Craig, J. C., et al. (2007a) Accuracy of procalcitonin for sepsis diagnosis in critically ill patients: systematic review and meta-analysis, Lancet Infect. Dis. 7, 210-217
Tang, B. M. P., McLean, A. S., Dawes, L W., Huang, S. J., Lin, R. C. Y., (2007b) The Use of Gene-Expression Profiling to Identify Candidate Genes In Human Sepsis.
American Journal of Respiratory and Critical Care Medicine 176(7), 676-684 The NCBI handbook [Internet]. Bethesda (MD): National Library of Medicine (US), National Center for Biotechnology Information; 2002 Oct.
US Patent Application 20060246495 “Diagnosis of sepsis”
U.S. Pat. No. 6,960,439 “Identification, monitoring and treatment of disease and characterization of biological condition using gene expression profiles”
Vandesompele, J., Preter, De K., Pattyn, F., et al. (2002) Accurate normalisation of real-time quantitative PCR data by geometric averaging of multiple internal control genes. Genome Biology 3(7), research0034.1-0034.11
Valasek, M. A., Repa, J. J., (2005) The power of real-time PCR. Advan. Physiol. Educ., 29, 151-159
Vapnik, V., (1999). The Nature of Statistical Learning Theory. Springer, New York.
Whitcombe, D., Theaker, J., Guy, S. P., et al. (1999) Detection of PCR products using selfprobing amplicons and fouoreschence. Nat. Biotechnol., 17, 904-907
WO 2006/100203 use of gene activity in vitro classifiers for the classification of gene expression profiles of patients with infectious/noninfectious
WO 2004/087949 methods for the detection of acute, generalized inflammatory status (SIRS), sepsis, systemic infections and sepsis-like conditions
WO 2005/083115 methods for the detection of sepsis
WO 2005/106020 method for establishment of criteria for predicting disease progression in sepsis
WO 2006/042581 method to distinguish between noninfectious and infectious causes of multiorgan failure
WO 2007/144105 method for detection of local inflammation of fever of unknown etiology
WO 2007/124820 vitro method for monitoring postoperative changes after liver transplantation
WO 2003/084388 Early detection of sepsis
Wong, M L., Medrano, J. F., (2005) Real-time PCR for mRNA quantification. Biotechniques 39(1) 1-11
Zeni, F., Freeman, B, Natanson, C., (1997) Anti-inflammatory therapies to treat sepsis and septic shock: A reassessment. Crit. Care Med., 25(7), 1095-1100

1-19. (canceled)

20. A method for at least one of in vitro identification, early detection, differentiation, progress monitoring and evaluation of a pathophysiological condition, wherein said pathophysiological condition is selected from the group consisting of: SIRS, sepsis, sepsis-like conditions, septic shock, bacteremia, and infectious and non-infectious multiorgan failure, the method comprising the following steps:
a) isolating sample nucleic acids from a sample derived from a patient;
b) selecting at least one, preferably at least three, polynucleotides of which the activity level is an indicator of said pathophysiological condition of a patient, wherein said polynucleotides are selected from the group consisting of M2, M3, M4, M6, M7, M8, M9, M10, M12, M13, M15, M16 and M17, or the isoforms, gene loci, transcripts and/or fragments of said polynucleotides with a length of at least five nucleotides, and forming therewith at least one biomarker, or multi-gene biomarker, diagnostic test, wherein the polynucleotide(s) are selected from the following table:

| | Transcript variants/cis- | Accession | SEQ |
|---|---|---|---|
| Polynucleotide | regulatory sequences | Number | ID NO: |
| M2 | M2_1 | NM_001031700 | 1 |
| | M2_2 | NM_016613 | 2 |
| | M2_3 | NM_001128424 | 3 |
| M4 | M4_1 | NM_203330 | 4 |
| | M4_2 | NM_000611 | 5 |
| | M4_3 | NM_203329 | 6 |
| | M4_4 | NM_203331 | 7 |
| | M4_5 | NM_001127223 | 8 |
| | M4_6 | NM_001127225 | 9 |
| | M4_7 | NM_001127226 | 10 |
| | M4_8 | NM_001127227 | 11 |
| M6 | M6_1 | NM_001831 | 12 |
| | M6_2 | NM_203339 | 13 |
| M7 | M7_1 | NM_031311 | 14 |
| | M7_2 | NM_019029 | 15 |
| M9 | M9 | NM_006682 | 16 |
| M10 | M10 | NM_033554 | 17 |
| M15 | M15_1 | NM_003580 | 18 |
| | M15_2 | NM_001144772 | 19 |
| M3 | M3_A | NM_001123041 | 20 |
| | M3_B | NM_001123396 | 21 |
| M8 | M8 | NM_025209 | 22 |
| | M8_cis | AI807985 | 23 |
| M12 | M12 | NM_002185 | 24 |
| | M12_cis | DB155561 | 25 |
| M13 | M13 | NM_001080394 | 26 |
| M16 | M16 | NM_003268 | 27 |
| M17 | M17 | NM_182491 | 28 |

c) selecting at least one internal reference gene and determining the gene activity level thereof,
d) testing the nucleic acids obtained from the sample of the patient for the activity level of said at least one, preferably at least three, polynucleotides using the diagnostic test of step b),
e) normalizing the gene activity value(s) of step d) against the reference gene activity of step c) to form one value, or one combined value from said at least 3 specific gene activities of the multi-gene biomarkers, indicative of said pathophysiological condition, and diagnosing said patient as having said pathophysiological condition.

21. The method of claim 20, wherein the reference gene is selected from polynucleotides of the group consisting of R1, R2 and R3 and/or their isoforms and/or their gene loci and/or their transcripts and/or fragments with a length of at least 5 nucleotides thereof, wherein the reference genes are selected from following table:

| | Transcript variants/ | | |
|---|---|---|---|
| Reference | cis-regulatory | | |
| gene | sequences | Accession Number | SEQ ID NO: |
| R1 | R1_A | NM_001228 | 29 |
| | R1_B | NM_033355 | 30 |
| | R1_C | NM_033356 | 31 |
| | R1_E | NM_033358 | 32 |
| | R1_F | NM_001080124 | 33 |
| | R1_G | NM_001080125 | 34 |
| R2 | R2_1 | NM_002209 | 35 |
| | R2_2 | NM_001114380 | 36 |
| R3 | R3 | NM_003082 | 37 |

22. The method of claim 20, wherein the polynucleotide sequences are selected from the group consisting of: loci, sense or antisense strands of pre-mRNA or mRNA, small RNA, and transposable elements detection of gene expression profiles.

23. The method of claim 20, wherein in b) the gene activity of from 4 to 13 polynucleotides is determined.

24. The method according to claim 20, wherein 4, 5, 6, 7, 8, 9, 10, 11 or 12 polynucleotides, or all 13 polynucleotides are used, wherein the polynucleotides are selected from the group consisting of: M2, M3, M4, M6, M7, M8, M9, M10, M12, M13, M15, M16 and M17 and/or their isoforms and/or their gene loci and/or their transcripts and/or fragments thereof with a length of at least five nucleotides, and wherein the number of polynucleotides preferably is 7.

25. A multiplex assay tool for in vitro identification and/or early detection and/or differentiation and/or progress monitoring and/or assessment of pathophysiological conditions of a patient, wherein the pathophysiological condition is selected the group consisting of: SIRS, sepsis and its degrees of severity; sepsis conditions, septic shock, bacteremia, infective/non-infectious multiple organ failure,
wherein said multiplex assay tool comprises at least three polynucleotides selected from the group consisting of: M2, M3, M4, M6, M7, M8, M9, M10, M12, M13, M15, M16 and M17 and/or their isoforms and/or their gene loci and/or their transcripts and/or fragments thereof with a length of at least five nucleotides, and wherein the polynucleotides are selected from the following table:

| | Transcript variants/cis- | Accession | SEQ |
|---|---|---|---|
| Polynucleotide | regulatory sequences | Number | ID NO: |
| M2 | M2_1 | NM_001031700 | 1 |
| | M2_2 | NM_016613 | 2 |
| | M2_3 | NM_001128424 | 3 |
| M4 | M4_1 | NM_203330 | 4 |
| | M4_2 | NM_000611 | 5 |
| | M4_3 | NM_203329 | 6 |
| | M4_4 | NM_203331 | 7 |
| | M4_5 | NM_001127223 | 8 |
| | M4_6 | NM_001127225 | 9 |
| | M4_7 | NM_001127226 | 10 |
| | M4_8 | NM_001127227 | 11 |
| M6 | M6_1 | NM_001831 | 12 |
| | M6_2 | NM_203339 | 13 |
| M7 | M7_1 | NM_031311 | 14 |
| | M7_2 | NM_019029 | 15 |
| M9 | M9 | NM_006682 | 16 |
| M10 | M10 | NM_033554 | 17 |
| M15 | M15_1 | NM_003580 | 18 |
| | M15_2 | NM_001144772 | 19 |
| M3 | M3_A | NM_001123041 | 20 |
| | M3_B | NM_001123396 | 21 |
| M8 | M8 | NM_025209 | 22 |
| | M8_cis | AI807985 | 23 |
| M12 | M12 | NM_002185 | 24 |
| | M12_cis | DB155561 | 25 |
| M13 | M13 | NM_001080394 | 26 |
| M16 | M16 | NM_003268 | 27 |
| M17 | M17 | NM_182491 | 28 |

26. The multiplex assay tool according to claim 25, wherein the multi-gene biomarker is the combination of several polynucleotide sequences, in particular gene sequences, wherein activities of the gene sequences are determined, and on the basis of their activities, using an interpretation function, a classification is carried out and/or an index is created with the data of the gene activities.

27. The multiplex assay tool according to claim 25, wherein the gene activity is determined by enzymatic methods, in particular amplification technique, preferably polymerase chain reaction (PCR), preferably real-time PCR, especially probe based procedures such as Taq-Man, Scorpions, Molecular Beacons; and/or by hybridization procedures, in particular those on microarrays; and/or direct mRNA detection, in particular sequencing or mass spectrometry; and/or isothermal amplification.

28. The multiplex assay tool according to claim 25, wherein an index is formed for each specific gene activity, which after the appropriate calibration provides a measure of the severity and/or the course of the pathophysiological condition, wherein the index is adapted to be displayed on an easily interpretable scale.

29. The multiplex assay tool according to claim 25, wherein for the establishment of the gene activity data, such specific gene loci, sense and/or antisense strands of pre-mRNA and/or mRNA, small RNA, especially scRNA, snoRNA, micro RNA, siRNA, dsRNA, ncRNA or elements, genes and/or gene fragments of a length of at least five nucleotides are used, which have a sequence homology of at least about 10%, especially about 20%, preferably about 50%, more preferably about 80%, to polynucleotide sequences M2, M3, M4, M6, M7, M8, M9, M10, M12, M13, M15, M16 and M17.

30. The multiplex assay tool according to claim 25, wherein the pathophysiological condition is selected the group consisting of: SIRS, sepsis and its degrees of severity; sepsis conditions, septic shock, bacteremia, infective/non-infectious multiple organ failure, early detection of these conditions; Focus Control, control of surgical rehabilitation of the infection focus; responders/non-responders to a particular therapy, treatment monitoring; distinction between infectious and non-infectious etiology of systemic reactions of the organism, such as SIRS, sepsis, postoperative complications, chronic and/or acute organ dysfunction, shock reaction, inflammatory response and/or trauma.

31. The multiplex assay tool according to claim 25, wherein the sample nucleic acid is RNA, in particular, total RNA or mRNA, or DNA, especially cDNA.

32. The multiplex assay tool according to claim 31, wherein, in order to assess the pathophysiological condition, in addition to at least one of the polynucleotides selected from the group consisting of: M2, M3, M4, M6, M7, M8, M9, M10, M12, M13, M15, M16 and M17 and/or their isoforms and/or their gene loci and/or their transcripts and/or fragments thereof with a length of at least five nucleotides, and wherein at least one additional marker is used, which is selected the group consisting of: clinical laboratory parameters, especially procalcitonin (PCT), C-reactive protein (CRP), leukocyte count, cytokines, interleukins and genetic, transcriptomic and proteomic markers.

33. A method for the production of software for defining at least one pathophysiologic condition and/or a research issue and/or as a tool for diagnostic purposes and/or patient data management systems, particularly for the use of patient classification and as an inclusion criterion for clinical studies, the method comprising
[if you would like such a claim, please draft it based on the research and statistical methods in the specification].

34. A primer for implementing the method according to claim 20, wherein the primer is selected according to the following table:

| Marker and | Primers for | |
|---|---|---|
| reference | quantitative PCR/ | |
| gene | resulting amplicon | SEQ ID NO: |
| M2 | M2-fw | 38 |
| | M2-rev | 39 |
| | M2-Amplikon | 40 |
| M4 | M4-fw | 41 |
| | M4-rev | 42 |
| | M4-Amplikon | 43 |
| M6 | M6-fw | 44 |
| | M6-rev | 45 |
| | M6-Amplikon | 46 |
| M7 | M7-fw | 47 |
| | M7-rev | 48 |
| | M7-Amplikon | 49 |
| M9 | M9-fw | 50 |
| | M9-rev | 51 |
| | M9-Amplikon | 52 |
| M10 | M10-fw | 53 |
| | M10-rev | 54 |
| | M10-Amplikon | 55 |
| M15 | M15-fw | 56 |
| | M15-rev | 57 |
| | M15-Amplikon | 58 |
| M3 | M3-fw | 59 |
| | M3-rev | 60 |
| | M3-Amplikon | 61 |
| M8 | M8-fw | 62 |
| | M8-rev | 63 |
| | M8-Amplikon | 64 |
| M12 | M12-fw | 65 |
| | M12-rev | 66 |
| | M12-Amplikon | 67 |
| M13 | M13-fw | 68 |
| | M13-rev | 69 |
| | M13-Amplikon | 70 |
| M16 | M16-fw | 71 |
| | M16-rev | 72 |
| | M16-Amplikon | 73 |
| M17 | M17-fw | 74 |
| | M17-rev | 75 |
| | M17-Amplikon | 76 |
| R1 | R1-fw | 77 |
| | R1-rev | 78 |
| | R1-Amplikon | 79 |
| R2 | R2-fw | 80 |
| | R2-rev | 81 |
| | R2-Amplikon | 82 |
| R3 | R3-fw | 83 |
| | R3-rev | 84 |
| | R3-Amplikon | 85 |

35. A kit for performing the method according to claim 20, comprising at least one multi-gene biomarker, which includes a plurality of polynucleotide sequences which are selected from the group consisting of: M2, M3, M4, M6, M7, M8, M9, M10, M12, M13, M15, M16 and M17 and/or their isoforms and/or their gene loci and/or their transcripts and/or fragments with a length of at least 5 nucleotides thereof, wherein the polynucleotides are defined according to the following table:

| | Transcript variants/ | | |
|---|---|---|---|
| Markers and | cis-regulatory | Accession | SEQ |
| reference gene | sequences | Number | ID NO: |
| M2 | M2_1 | NM_001031700 | 1 |
| | M2_2 | NM_016613 | 2 |
| | M2_3 | NM_001128424 | 3 |
| M4 | M4_1 | NM_203330 | 4 |
| | M4_2 | NM_000611 | 5 |
| | M4_3 | NM_203329 | 6 |
| | M4_4 | NM_203331 | 7 |
| | M4_5 | NM_001127223 | 8 |
| | M4_6 | NM_001127225 | 9 |
| | M4_7 | NM_001127226 | 10 |
| | M4_8 | NM_001127227 | 11 |
| M6 | M6_1 | NM_001831 | 12 |
| | M6_2 | NM_203339 | 13 |
| M7 | M7_1 | NM_031311 | 14 |
| | M7_2 | NM_019029 | 15 |
| M9 | M9 | NM_006682 | 16 |
| M10 | M10 | NM_033554 | 17 |
| M15 | M15_1 | NM_003580 | 18 |
| | M15_2 | NM_001144772 | 19 |
| M3 | M3_A | NM_001123041 | 20 |
| | M3_B | NM_001123396 | 21 |
| M8 | M8 | NM_025209 | 22 |
| | M8_cis | AI807985 | 23 |
| M12 | M12 | NM_002185 | 24 |
| | M12_cis | DB155561 | 25 |
| M13 | M13 | NM_001080394 | 26 |
| M16 | M16 | NM_003268 | 27 |
| M17 | M17 | NM_182491 | 28 |

wherein the multi-gene biomarker specific to a pathophysiological condition of a patient and that such conditions include, which are selected the group consisting of: SIRS, sepsis and its degrees of severity; sepsis conditions, septic shock, bacteremia, infective/non-infectious multiple organ failure, early detection of these conditions; focus control, control of surgical rehabilitation of the infection focus; responders/non-responders to a particular therapy, treatment monitoring; distinction between infectious and non-infectious etiology of systemic reactions of the organism, such as SIRS, sepsis, postoperative complications, chronic and/or acute organ dysfunction, shock response, inflammatory response and/or trauma.

36. The kit according to claim 35, wherein the polynucleotide sequences also include gene loci, sense and/or antisense strands of pre-mRNA and/or mRNA, small RNA, especially scRNA, snoRNA, micro RNA, siRNA, dsRNA, ncRNA or transposable elements.

37. The kit according to claim 35, wherein it includes at least the reference gene which is selected from the group consisting of: R1, R2 and R3 and/or their isoforms and/or their gene loci and/or their transcripts and/or fragments thereof with a length of at least five nucleotides, wherein the reference genes are defined according to the following table:

| | Transcript variants/ | | |
|---|---|---|---|
| Reference | cis-regulatory | | |
| gene | sequences | Accession Number | SEQ ID NO: |
| R1 | R1_A | NM_001228 | 29 |
| | R1_B | NM_033355 | 30 |
| | R1_C | NM_033356 | 31 |
| | R1_E | NM_033358 | 32 |
| | R1_F | NM_001080124 | 33 |
| | R1_G | NM_001080125 | 34 |
| R2 | R2_1 | NM_002209 | 35 |
| | R2_2 | NM_001114380 | 36 |
| R3 | R3 | NM_003082 | 37 |
